(19) United States
(12) Patent Application Publication
(10) Pub. No.: US 2010/0056565 A1
(43) Pub. Date: Mar. 4, 2010

(54) Heterocyclic Derivatives as M3 Muscarinic Receptors

(75) Inventors: Barbara Giuseppina Avitabile, Essex (GB); Harry Finch, Essex (GB); Jamie David Knight, Essex (GB); Alan John Nadin, Essex (GB); Nicholas Charles Ray, Essex (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ARGENTA DISCOVERY LIMITED, Harlow, Essex (GB)

(21) Appl. No.: 12/523,609

(22) PCT Filed: Feb. 14, 2008
(86) PCT No.: PCT/GB2008/000519

§ 371 Date: Oct. 16, 2009

(30) Foreign Application Priority Data
Feb. 15, 2007 (GB) 0702994.5
Nov. 19, 2007 (GB) 0722678.9

(51) Int. Cl. A61K 31/439; C07D 453/02; A61P 11/06; A61P 11/04; A61P 11/08

(52) U.S. Cl. 514/305; 546/137

(57) ABSTRACT

This invention relates to M3 antagonists of formula (I) wherein R², R⁴, R⁵, R⁶, W, V, A, D, X, t, u and v are as defined herein; pharmaceutical compositions containing them; methods for their preparation; and their use in the treatment of diseases where enhanced M3 receptor activation is implicated.

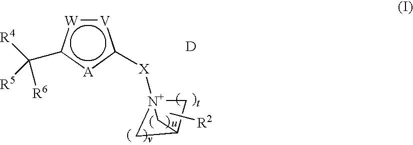

### FIELD OF THE INVENTION

This invention relates to heterocycles, pharmaceutical compositions, methods for their preparation and use in the treatment of diseases where enhanced M3 receptor activation is implicated.

### BACKGROUND TO THE INVENTION

Anti-cholinergic agents prevent the passage of, or effects resulting from the passage of, impulses through the parasympathetic nerves. This is a consequence of the ability of such compounds to inhibit the action of acetylcholine (Ach) by blocking its binding to the muscarinic cholinergic receptors.

There are five subtypes of muscarinic acetylcholine receptors (mAChRs), termed M1-M5, and each is the product of a distinct gene and each displays unique pharmacological properties. mAChRs are widely distributed in vertebrate organs, and these receptors can mediate both inhibitory and excitatory actions. For example, in smooth muscle found in the airways, bladder and gastrointestinal tract, M3 mAChRs mediate contractile responses (reviewed by Caulfield, 1993, Pharmac. Ther., 58, 319-379).

In the lungs, muscarinic receptors M1, M2 and M3 have been demonstrated to be important and are localized to the trachea, the bronchi, submucosal glands and parasympathetic ganglia (reviewed in Fryer and Jacoby, 1998, Am J Resp Crit Care Med., 158 (5 part 3) S154-160). M3 receptors on airway smooth muscle mediate contraction and therefore bronchoconstriction. Stimulation of M3 receptors localised to submucosal glands results in mucus secretion.

Increased signalling through muscarinic acetylcholine receptors has been noted in a variety of different pathophysiological states including asthma and COPD. In COPD, vagal tone may either be increased (Gross et al. 1989, Chest; 96:984-987) and/or may provoke a higher degree of obstruction for geometric reasons if applied on top of edematous or mucus-laden airway walls (Gross et al. 1984, Am Rev Respir Dis; 129:856-870). In addition, inflammatory conditions can lead to a loss of inhibitory M2 receptor activity which results in increased levels of acetylcholine release following vagal nerve stimulation (Fryer et al, 1999, Life Sci., 64, (6-7) 449-455). The resultant increased activation of M3 receptors leads to enhanced airway obstruction. Thus the identification of potent muscarinic receptor antagonists would be useful for the therapeutic treatment of those disease states where enhanced M3 receptor activity is implicated. Indeed, contemporary treatment strategies currently support regular use of M3 antagonist bronchodilators as first-line therapy for COPD patients (Pauwels et al. 2001, Am Rev Respir Crit Care Med; 163:1256-1276).

Incontinence due to bladder hypercontractility has also been demonstrated to be mediated through increased stimulation of M3 mAChRs. Thus M3 mAChR antagonists may be useful as therapeutics in these mAChR-mediated diseases.

Despite the large body of evidence supporting the use of anti-muscarinic receptor therapy for treatment of airway disease states, relatively few anti-muscarinic compounds are in use in the clinic for pulmonary indications. Thus, there remains a need for novel compounds that are capable of causing blockade at M3 muscarinic receptors, especially those compounds with a long duration of action, enabling a once-daily dosing regimen. Since muscarinic receptors are widely distributed throughout the body, the ability to deliver anticholinergic drugs directly to the respiratory tract is advantageous as it allows lower doses of the drug to be administered. The design and use of topically active drugs with a long duration of action and that are retained on the receptor or in the lung would allow reduction of unwanted side effects that could be seen with systemic administration of the same drugs.

Tiotropium (Spiriva™) is a long-acting muscarinic antagonist currently marketed for the treatment of chronic obstructive pulmonary disease, administered by the inhaled route.

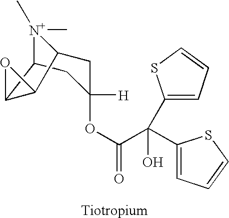

Additionally ipratropium is a muscarinic antagonist marketed for the treatment of COPD.

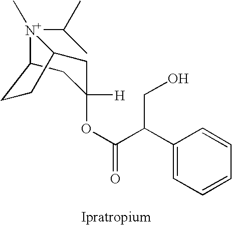

Chem. Pharm. Bull. 27 (12) 3149-3152 (1979) and J. Pharm. Sci 69 (5) 534-537 (1980) describe furyl derivatives as possessing atropine-like activities.

Med. Chem. Res 10 (9), 615-633 (2001) describes isoxazoles and Δ²-isoxazolines as muscarinic antagonists.

WO97/30994 describes oxadiazoles and thiadiazoles as muscarinic receptor antagonists.

EP0323864 describes oxadiazoles linked to a mono- or bicyclic ring as muscarinic receptor modulators.

### SUMMARY OF THE INVENTION

According to the invention, there is provided a compound of formula (I):

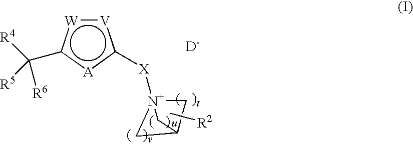

wherein
R² is a group H, —(Z)ₚ—R⁷, —Z—Y—R⁷ or —Y—R⁷;
p is 0 or 1;
R⁴ and R⁵ are independently selected from the group consisting of aryl, aryl-fused-heterocycloalkyl, heteroaryl, C₁-C₆-alkyl, and cycloalkyl;
R⁶ is —OH, C₁-C₆-alkyl, C₁-C₆-alkoxy, hydroxy-C₁-C₆-alkyl, nitrile, a group CONR¹R⁹ or a hydrogen atom;
one of W, V and A is N or NR¹¹; another of W, V and A is N, O, S or CR⁸; and the last one of W, V and A is N or CR⁸;
X is an C₁-C₄-alkylene, C₂-C₄-alkenylene or C₂-C₄-alkynylene group;
R⁷ is an C₁-C₆-alkyl, C₂-C₆-alkenyl, aryl, aryl-fused-cycloalkyl, aryl-fused-heterocycloalkyl, heteroaryl, aryl(C₁-C₈-alkyl)-, heteroaryl(C₁-C₈-alkyl)-, heterocycloalkyl or cycloalkyl group;
t, u and v are independently selected from 1, 2 or 3, with the proviso that t, u and v cannot all simultaneously be 1;
Z is a C₁-C₄-alkylene, C₂-C₄-alkenylene or C₂-C₄-alkynylene group;
Y is an oxygen atom, a group —OC(O)—, a group —N(H)C(O)— or a group —S(O)ₙ;
n is 0, 1 or 2;
R¹, R⁸, R⁹ and R¹¹ are, independently, a hydrogen atom or C₁-C₆-alkyl group; and
D⁻ is a pharmaceutically acceptable counter-ion;
wherein, unless otherwise specified, each occurrence of alkyl, alkenyl, heterocycloalkyl, aryl, aryl-fused-heterocycloalkyl, heteroaryl, cycloalkyl, alkoxy, alkylene, alkenylene, alkynylene or aryl-fused-cycloalkyl may be optionally substituted; and
wherein each alkenylene chain contains, where possible, up to 2 carbon-carbon double bonds and each alkynylene chain contains, where possible, up to 2 carbon-carbon triple bonds.

In one particular aspect the present invention provides a pharmaceutically acceptable salt of a compound of formula (I) as herein defined.

In another aspect the present invention provides a prodrug of a compound of formula (I) as herein defined, or a pharmaceutically acceptable salt thereof.

In yet another aspect the present invention provides an N-oxide of a compound of formula (I) as herein defined, or a prodrug or pharmaceutically acceptable salt thereof.

In a further aspect the present invention provides a solvate (such as a hydrate) of a compound of formula (I) as herein defined, or an N-oxide, prodrug or pharmaceutically acceptable salt thereof.

It will be appreciated that in the compounds of formula (I) above, the substituent R² can be attached to any carbon atom of the azabicyclic ring.

It will be appreciated that the carbon atom to which R⁴, R⁵ and R⁶ are attached can be an asymmetric centre so compounds of the invention may be in the form of single enantiomers or mixtures of enantiomers. In such cases, both enantiomers of the invention generally exhibit affinity at the M₃ receptor, although one enantiomer is generally preferred on criteria of potency at the M₃ receptor and/or selectivity against the M₂ receptor.

Compounds of the invention may be useful in the treatment or prevention of diseases in which activation of muscarinic receptors are implicated, for example the present compounds are useful for treating a variety of indications, including but not limited to respiratory-tract disorders such as chronic obstructive lung disease (also known as chronic obstructive pulmonary disease or COPD), chronic bronchitis of all types (including dyspnoea associated therewith), asthma (allergic and non-allergic; ‘wheezy-infant syndrome’), adult/acute respiratory distress syndrome (ARDS), chronic respiratory obstruction, bronchial hyperactivity, pulmonary fibrosis, pulmonary emphysema, and allergic rhinitis, exacerbation of airway hyperreactivity consequent to other drug therapy, particularly other inhaled drug therapy, pneumoconiosis (for example aluminosis, anthracosis, asbestosis, chalicosis, ptilosis, siderosis, silicosis, tabacosis and byssinosis);

gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollakiuria; motion sickness; and cardiovascular disorders such as vagally induced sinus bradycardia.

In another aspect a compound of present invention is useful in the treatment or prevention of respiratory-tract disorders such as chronic obstructive lung disease (also known as chronic obstructive pulmonary disease, COPD), chronic bronchitis of all types (including dyspnoea associated therewith), asthma (allergic and non-allergic; ‘wheezy-infant syndrome’), adult/acute respiratory distress syndrome (ARDS), chronic respiratory obstruction, bronchial hyperactivity, pulmonary fibrosis, pulmonary emphysema, and allergic rhinitis, exacerbation of airway hyperreactivity consequent to other drug therapy, particularly other inhaled drug therapy or pneumoconiosis (for example aluminosis, anthracosis, asbestosis, chalicosis, ptilosis, siderosis, silicosis, tabacosis and byssinosis).

For treatment of respiratory conditions, administration by inhalation will often be preferred, and in such cases administration of compounds (I) which are quaternary ammonium salts will often be preferred. In many cases, the duration of action of quaternary ammonium salts of the invention administered by inhalation is may be more than 12, or more than 24 hours for a typical dose. For treatment of gastrointestinal-tract disorders and cardiovascular disorders, administration by the parenteral route, usually the oral route, may be preferred.

Another aspect of the invention is a pharmaceutical composition comprising a compound of the invention and a pharmaceutically acceptable carrier, diluent or excipient.

Another aspect of the invention is the use of a compound of the invention for the manufacture of a medicament for the treatment or prevention of a disease or condition in which muscarinic M3 receptor activity is implicated. Diseases or conditions in which muscarinic M3 receptor activity is implicated include respiratory-tract disorders, gastrointestinal-tract disorders and cardiovascular disorders. Specific examples of such diseases and conditions include those listed above.

Another aspect of the invention provides a compound of the invention for the treatment or prevention of a disease or condition in which muscarinic M3 receptor activity is implicated. Diseases or conditions in which muscarinic M3 receptor activity is implicated include respiratory-tract disorders, gastrointestinal-tract disorders and cardiovascular disorders. Specific examples of such diseases and conditions include those listed above.

Another aspect of the invention provides a method of treatment of a disease or condition in which M3 muscarinic receptor activity is implicated comprising administration to a subject in need thereof a therapeutically effective amount of a compound of the invention. Diseases or conditions in which muscarinic M3 receptor activity is implicated include respiratory-tract disorders, gastrointestinal-tract disorders and cardiovascular disorders. Specific examples of such diseases and conditions include those listed above.

Another aspect of the invention provides a compound of the invention for use in therapy.

### DESCRIPTION OF DEFINITIONS

Unless otherwise qualified in the context in which they are used, the following terms have the following meanings when used herein:

“Acyl” means a —CO-alkyl group in which the alkyl group is as described herein. Exemplary acyl groups include —COCH₃ and —COCH(CH₃)₂.

“Acylamino” means a —NR-acyl group in which R and acyl are as described herein. Exemplary acylamino groups include —NHCOCH₃ and —N(CH₃)COCH₃.

“Alkoxy” and “alkyloxy” means an —O-alkyl group in which alkyl is as described below. Exemplary alkoxy groups include methoxy (—OCH₃) and ethoxy (—OC₂H₅).

“Alkoxycarbonyl” means a —COO-alkyl group in which alkyl is as defined below. Exemplary alkoxycarbonyl groups include methoxycarbonyl and ethoxycarbonyl.

“Alkyl” as a group or part of a group refers to a straight or branched chain saturated hydrocarbon group having from 1 to 12, typically 1 to 6, carbon atoms, in the chain. Exemplary alkyl groups include methyl, ethyl, 1-propyl and 2-propyl.

“Alkenyl” as a group or part of a group refers to a straight or branched chain hydrocarbon group having from 2 to 12, typically 2 to 6, or 2 to 4 carbon atoms and one or more carbon-carbon double bonds in the chain. Exemplary alkenyl groups include ethenyl, 1-propenyl, and 2-propenyl.

“Alkylamino” means a —NH-alkyl group in which alkyl is as defined above. Exemplary alkylamino groups include methylamino and ethylamino.

“Alkylene” means an -alkyl-group in which alkyl is as defined previously. Exemplary alkylene groups include —CH₂—, —(CH₂)₂— and —C(CH₃)HCH₂—.

“Alkenylene” means an -alkenyl-group in which alkenyl is as defined previously. Exemplary alkenylene groups include —CH═CH—, —CH═CHCH₂—, and —CH₂CH═CH—.

“Alkynylene” means an -alkynyl-group in which -alkynyl- refers to a straight or branched chain hydrocarbon group having from 2 to 12, typically 2 to 6, or 2 to 4 carbon atoms and one or more carbon-carbon triple bond in the chain. Exemplary alkynylene groups include ethynyl and propargyl.

“Alkylsulfinyl” means a —SO-alkyl group in which alkyl is as defined above. Exemplary alkylsulfinyl groups include methylsulfinyl and ethylsulfinyl.

“Alkylsulfonyl” or “sulfonyl” each means a —SO₂-alkyl group in which alkyl is as defined above. Exemplary alkylsulfonyl groups include methylsulfonyl and ethylsulfonyl.

“Alkylthio” means a —S-alkyl group in which alkyl is as defined above. Exemplary alkylthio groups include methylthio and ethylthio.

“Aminoacyl” means a —CO—NRR group in which R is as herein described. Exemplary aminoacyl groups include —CONH₂ and —CONHCH₃.

“Aminoalkyl” means an alkyl-NH₂ group in which alkyl is as previously described. Exemplary aminoalkyl groups include —CH₂NH₂.

“Aminosulfonyl” means a —SO₂—NRR group in which R is as herein described. Exemplary aminosulfonyl groups include —SO₂NH₂ and —SO₂NHCH₃.

“Aryl” as a group or part of a group denotes an optionally substituted monocyclic or multicyclic aromatic carbocyclic moiety of from 6 to 14 carbon atoms, typically from 6 to 10 carbon atoms, such as phenyl or naphthyl. Phenyl is a typical aryl group. The aryl group, specifically a phenyl group, may be substituted by one or more substituent groups.

“Arylalkyl” means an aryl-alkyl-group in which the aryl and alkyl moieties are as previously described. Typical arylalkyl groups contain a C₁₋₄ alkyl moiety. Exemplary arylalkyl groups include benzyl, phenethyl and naphthlenemethyl.

“Arylalkyloxy” means an aryl-alkyloxy-group in which the aryl and alkyloxy moieties are as previously described. Typical arylalkyloxy groups contain a C₁₋₄ alkyl moiety. Exemplary arylalkyl groups include benzyloxy.

“Aryl-fused-cycloalkyl” means a monocyclic aryl ring, such as phenyl, fused to a cycloalkyl group, in which the aryl and cycloalkyl are as described herein. Exemplary aryl-fused-cycloalkyl groups include tetrahydronaphthyl and indanyl. The aryl and cycloalkyl rings may each be substituted by one or more substituent groups. The aryl-fused-cycloalkyl group may be attached to the remainder of the compound by any available carbon atom.

“Aryl-fused-heterocycloalkyl” means a monocyclic aryl ring, such as phenyl, fused to a heterocycloalkyl group, in which the aryl and heterocycloalkyl are as described herein. Exemplary aryl-fused-heterocycloalkyl groups include tetrahydroquinolinyl, indolinyl, benzodioxinyl, benxodioxolyl, dihydrobenzofuranyl and isoindolonyl. The aryl and heterocycloalkyl rings may each be substituted by one or more substituent groups. The aryl-fused-heterocycloalkyl group may be attached to the remainder of the compound by any available carbon or nitrogen atom.

“Aryloxy” means an —O-aryl group in which aryl is described above. Exemplary aryloxy groups include phenoxy.

“Cyclic amine” means an optionally substituted 3 to 8 membered monocyclic cycloalkyl ring system where one of the ring carbon atoms is replaced by nitrogen, and which may optionally contain an additional heteroatom selected from O, S or NR (where R is as described herein). Exemplary cyclic amines include pyrrolidine, piperidine, morpholine, piperazine and N-methylpiperazine. The cyclic amine group may be substituted by one or more substituent groups.

“Cycloalkyl” means an optionally substituted saturated monocyclic or bicyclic ring system of from 3 to 12 carbon atoms, typically from 3 to 8 carbon atoms, and more typically from 3 to 6 carbon atoms. Exemplary monocyclic cycloalkyl rings include cyclopropyl, cyclopentyl, cyclohexyl and cycloheptyl. The cycloalkyl group may be substituted by one or more substituent groups.

“Dialkylamino” means a —N(alkyl)₂ group in which alkyl is as defined above. Exemplary dialkylamino groups include dimethylamino and diethylamino.

“Halo” or “halogen” means fluoro, chloro, bromo, or iodo. Typical are fluoro or chloro.

“Haloalkoxy” means an —O-alkyl group in which the alkyl is substituted by one or more halogen atoms. Exemplary haloalkyl groups include trifluoromethoxy and difluoromethoxy.

“Haloalkyl” means an alkyl group which is substituted by one or more halo atoms. Exemplary haloalkyl groups include trifluoromethyl.

“Heteroaryl” as a group or part of a group denotes an optionally substituted aromatic monocyclic or multicyclic organic moiety of from 5 to 14 ring atoms, typically from 5 to 10 ring atoms, in which one or more of the ring atoms is/are element(s) other than carbon, for example nitrogen, oxygen or sulfur. Examples of such groups include benzimidazolyl, benzoxazolyl, benzothiazolyl, benzofuranyl, benzothienyl, furyl, imidazolyl, indolyl, indolizinyl, isoxazolyl, isoquinolinyl, isothiazolyl, oxazolyl, oxadiazolyl, pyrazinyl, pyridazinyl, pyrazolyl, pyridyl, pyrimidinyl, pyrrolyl, quinazolinyl, quinolinyl, tetrazolyl, 1,3,4-thiadiazolyl, thiazolyl, thienyl and triazolyl groups. The heteroaryl group may be substituted by one or more substituent groups. The heteroaryl group may be attached to the remainder of the compound of the invention by any available carbon or nitrogen atom.

“Heteroarylalkyl” means a heteroaryl-alkyl-group in which the heteroaryl and alkyl moieties are as previously described. Typical heteroarylalkyl groups contain a lower alkyl moiety. Exemplary heteroarylalkyl groups include pyridylmethyl.

“Heteroarylalkyloxy” means a heteroaryl-alkyloxy-group in which the heteroaryl and alkyloxy moieties are as previously described. Typical heteroarylalkyloxy groups contain a lower alkyl moiety. Exemplary heteroarylalkyloxy groups include pyridylmethyloxy.

“Heteroaryloxy” means a heteroaryloxy-group in which the heteroaryl is as previously described. Exemplary heteroaryloxy groups include pyridyloxy.

“Heterocycloalkyl” means: (i) an optionally substituted cycloalkyl group of from 4 to 8 ring members which contains one or more heteroatoms selected from O, S or NR; (ii) a cycloalkyl group of from 4 to 8 ring members which contains CONR or CONRCO (examples of such groups include succinimidyl and 2-oxopyrrolidinyl). The heterocycloalkyl group may be substituted by one or more substituent groups. The heterocycloalkyl group may be attached to the remainder of the compound by any available carbon or nitrogen atom.

“Lower alkyl” as a group means unless otherwise specified, an aliphatic hydrocarbon group which may be straight or branched having 1 to 4 carbon atoms in the chain, i.e. methyl, ethyl, propyl (propyl or isopropyl) or butyl (butyl, isobutyl or tert-butyl).

“Sulfonylamino” means a —NR-sulfonyl group in which R and sulfonyl are as described herein. Exemplary sulfonylamino groups include —NHSO₂CH₃.

A substituent designation R in any of the above definitions means hydrogen, alkyl, aryl, or heteroaryl as described herein, and when two R groups are present on a group (for example on —SO₂—NRR) then the R groups can be the same or different.

“Pharmaceutically acceptable salt” means a physiologically or toxicologically tolerable salt and includes, when appropriate, pharmaceutically acceptable base addition salts, pharmaceutically acceptable acid addition salts, and pharmaceutically acceptable quaternary ammonium salts. For example (i) where a compound of the invention contains one or more acidic groups, for example carboxy groups, pharmaceutically acceptable base addition salts that may be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that may be formed include hydrochlorides, hydrobromides, sulfates, phosphates, acetates, citrates, lactates, tartrates, mesylates, napadisylates (naphthalene-1,5-disulfonates or naphthalene-1-(sulfonic acid)-5-sulfonates), edisylates (ethane-1,2-disulfonates or ethane-1-(sulfonic acid)-2-sulfonates), maleates, fumarates, succinates and the like; (iii) the compounds of the present invention contain a quaternary ammonium group, thus acceptable counter-ions may be, for example, chlorides, bromides, sulfates, methanesulfonates, benzenesulfonates, toluenesulfonates (tosylates), napadisylates (naphthalene-1,5-disulfonates or naphthalene-1-(sulfonic acid)-5-sulfonates), edisylates (ethane-1,2-disulfonates or ethane-1-(sulfonic acid)-2-sulfonates), isethionates (2-hydroxyethylsulfonates), xinafoates, p-acetamidobenzoates, phosphates, acetates, citrates, lactates, tartrates, maleates, fumarates, acetamidobenzoates, succinates and the like; wherein the number of quaternary ammonium species balances the pharmaceutically acceptable counter-ion D- such that compound of formula (I) has no net charge.

It will be understood that, as used herein, references to the compounds of the invention are meant to also include the pharmaceutically acceptable salts.

“Prodrug” refers to a compound which is convertible in vivo by metabolic means (e.g. by hydrolysis, reduction or oxidation) to a compound of the invention. Suitable groups for forming pro-drugs are described in ‘The Practice of Medicinal Chemistry, 2^{nd} Ed. pp 561-585 (2003) and in F. J. Leinweber, Drug Metab. Res., 18, 379. (1987)

It will be understood that, as used in herein, references to the compounds of the invention are meant to also include the prodrug forms.

“Saturated” pertains to compounds and/or groups which do not have any carbon-carbon double bonds or carbon-carbon triple bonds.

The cyclic groups referred to above, namely, aryl, heteroaryl, cycloalkyl, aryl-fused-cycloalkyl, heterocycloalkyl, aryl-fused-heterocycloalkyl and cyclic amine are unsubstituted or substituted by one or more of the same or different substituent groups. Examples of specific optional substituents include —Cl, —F, —CH₃, —OCH₃, —OH, —CN, —COOCH₃, —CONH₂, —SO₂NH₂, —SO₂N(CH₃)₂. More generally the substituents can be divided into two classes:

(a) a first class of substituent includes acyl (e.g. —COCH₃), alkoxy (e.g., —OCH₃), alkoxycarbonyl (e.g. —COOCH₃), alkylamino (e.g. —NHCH₃), alkylsulfinyl (e.g. —SOCH₃), alkylsulfonyl (e.g. —SO₂CH 3), alkylthio (e.g. —SCH₃), —NH₂, aminoacyl (e.g. —CON(CH₃)₂), aminoalkyl (e.g. —CH₂NH₂), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), —OH, —CHO, —COOH, —NO₂, aminoacyl (e.g. —CONH₂, —CONHCH₃), aminosulfonyl (e.g. —SO₂NH₂, —SO₂NHCH₃), acylamino (e.g. —NHCOCH₃) and sulfonylamino (e.g. —NHSO₂CH₃); and

(b) a second class of substituent includes arylalkyl (e.g. —CH₂Ph or —CH₂—CH₂-Ph), aryl, heteroaryl, heterocycloalkyl, heteroarylalkyl, cyclic amine (e.g. morpholine), aryloxy, heteroaryloxy, arylalkyloxy (e.g. benzyloxy) and heteroarylalkyloxy, the cyclic part of any of which being optionally substituted by any of the first class of substituent referred to above (for example alkoxy, haloalkoxy, halogen, alkyl and haloalkyl).

Alkyl, alkoxy and alkenyl groups may be optionally substituted. Suitable optional substituent groups for alkyl and alkenyl include alkoxy (e.g., —OCH₃), alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), —OH, —CHO, and —NO₂. Suitable optional substituent groups for alkoxy include alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), —OH, —CHO, and —NO₂.

Alkylene or alkenylene groups may be optionally substituted. Suitable optional substituent groups include alkoxy (e.g., —OCH₃), alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), —OH, —CHO, and —NO₂.

Compounds of the invention may exist in one or more geometrical, optical, enantiomeric, diastereomeric and tautomeric forms, including but not limited to cis- and trans-forms, E- and Z-forms, R—, S— and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers may be prepared by the application of adaptation of known methods (e.g. asymmetric synthesis).

The present invention further comprises a subset of compounds formula (I) wherein:

R⁴ and R⁵ are independently selected from the group consisting of aryl, heteroaryl, C₁-C₆-alkyl, and cycloalkyl;

### R⁶ is —OH; and

R⁷ is aryl, heteroaryl or heterocycloalkyl,
wherein aryl, heteroaryl, cycloalkyl and heterocycloalkyl are as previously defined.

The present invention further comprises another subset of compounds formula (I) wherein:

R² is selected from —(Z)ₚ—R⁷, —Z—Y—R⁷ and —Y—R⁷;
p is 1;
R⁴ and R⁵ are independently selected from the group consisting of aryl and cycloalkyl;

### R⁶ is —OH;

W is N, one of V and A is N, O or S, and the last one of V and A is N or CR⁸;
X is C₁-C₄-alkylene;
R⁷ is C₂-C₆-alkenyl, aryl, heteroaryl, aryl(C₁-C₈-alkyl)-, or heteroaryl(C₁-C₈-alkyl)-;
t, u and v are 2;
Z is a C₁-C₄-alkylene;
Y is an oxygen atom or a group —S(O)ₙ; and
n is 0;
wherein aryl, heteroaryl, cycloalkyl and heterocycloalkyl are as previously defined.

The present invention further comprises compounds of formula (I):

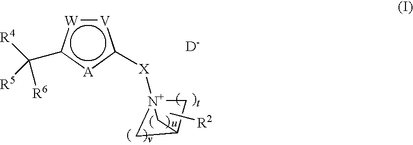

wherein
R² is a group H, —(Z)ₚ—R⁷, —Z—Y—R⁷ or —Y—R⁷;
p is 0 or 1;
R⁴ and R⁵ are independently selected from the group consisting of aryl, aryl-fused-heterocycloalkyl, heteroaryl, C₁-C₆-alkyl, and cycloalkyl;
R⁶ is —OH, C₁-C₆-alkyl, C₁-C₆-alkoxy, hydroxy-C₁-C₆-alkyl, nitrile, a group CONR¹R⁹ or a hydrogen atom;
one of W, V and A is N or NR¹¹; another of W, V and A is N, O, S or CR⁸; and the last one of W, V and A is N or CR⁸;
X is an C₁-C₄-alkylene, C₂-C₄-alkenylene or C₂-C₄-alkynylene group;
R⁷ is an C₁-C₆-alkyl, C₂-C₆-alkenyl, aryl, aryl-fused-cycloalkyl, aryl-fused-heterocycloalkyl, heteroaryl, aryl(C₁-C₈-alkyl)-, heteroaryl(C₁-C₈-alkyl)-, heterocycloalkyl or cycloalkyl group;
t, u and v are independently selected from 1, 2 or 3, with the proviso that t, u and v cannot all simultaneously be 1;
Z is a C₁-C₄-alkylene, C₂-C₄-alkenylene or C₂-C₄-alkynylene group;
Y is an oxygen atom, a group —OC(O)—, a group —N(H)C(O)— or a group —S(O)ₙ;
n is 0, 1 or 2;
R¹, R⁸, R⁹ and R¹¹ are, independently, a hydrogen atom or C₁-C₆-alkyl group; and
D⁻ is a pharmaceutically acceptable counter-ion;
wherein, each occurrence of each occurrence of alkyl may be optionally substituted with one or more substituent groups chosen from C₁-C₆-haloalkyl, C₁-C₆-haloalkoxy, CN and halo; and each occurrence of alkenyl, heterocycloalkyl, aryl, aryl-fused-heterocycloalkyl, heteroaryl, cycloalkyl, alkoxy, alkylene, alkenylene, alkynylene or aryl-fused-cycloalkyl may be optionally substituted with one or more substituent groups chosen from C₁-C₆-alkyl, C₁-C₆-haloalkyl, C₁-C₆-haloalkoxy, CN and halo; and
wherein each alkenylene chain contains, where possible, up to 2 carbon-carbon double bonds and each alkynylene chain contains, where possible, up to 2 carbon-carbon triple bonds.

The present invention also encompasses the following alternative embodiments and combinations thereof:

### The Group R²

In one alternative embodiment, the present invention provides compounds of formula (I) wherein R² is a group —(Z)ₚ—R⁷, p is 1 and —Z— is a straight or branched alkylene radical linking the azabicyclic ring and —R⁷ by a chain of up to 4, for example up to 2, carbon atoms. In these cases, R⁷ is typically a cyclic lipophilic group such as phenyl, benzyl, dihydrobenzofuryl or phenylethyl (wherein the phenyl rings are optionally substituted as described herein).

In another alternative embodiment, the present invention provides compounds of formula (I) wherein R² is a group —Y—R⁷ and Y is an oxygen or sulfur atom. In these cases, R⁷ is typically a cyclic lipophilic group such as phenyl, benzyl, dihydrobenzofuryl or phenylethyl (wherein the phenyl rings are optionally substituted as described herein).

In yet another alternative embodiment, the present invention provides compounds of formula (I) wherein R² is selected from —(Z)ₚ—R⁷, —Z—Y—R⁷ and —Y—R⁷.

In yet another alternative embodiment, the present invention provides compounds of formula (I) wherein R² is selected from —Z—Y—R⁷ and —Y—R⁷.

In a further alternative embodiment, the present invention provides compounds of formula (I) wherein R² is —(Z)ₚ—R⁷.

In a yet further alternative embodiment, the present invention provides compounds of formula (I) wherein R² is —Y—R⁷.

In a yet further still alternative embodiment, the present invention provides compounds of formula (I) wherein R² is H.

### The Groups R⁴, R⁵ and R⁶

In one alternative embodiment, the present invention provides a compound of formula (I) wherein R⁴ and R⁵ are, independently, aryl (such as phenyl), C₄-C₈ cycloalkyl (such as cyclopentyl or cyclohexyl) or heteroaryl (such as thienyl).

In another alternative embodiment, the present invention provides a compound of formula (I) wherein R⁴ is aryl (such as phenyl) or heteroaryl (such as thienyl).

In a further alternative embodiment, the present invention provides a compound of formula (I) wherein R⁵ is C₄-C₈ cycloalkyl (such as cyclopentyl or cyclohexyl) or heteroaryl (such as thienyl).

In a yet further alternative embodiment, the present invention provides a compound of formula (I) wherein R⁴ is aryl (for example phenyl) and R is C₄-C₇ cycloalkyl (for example cyclopentyl or cyclohexyl).

In one alternative embodiment, the present invention provides a compound of formula (I) wherein R⁶ is hydroxy, C₁-C₄ alkyl (such as methyl), C₁-C₄ alkoxy (such as methoxy) or nitrile.

In another alternative embodiment, the present invention provides a compound of formula (I) wherein R⁶ is selected from —OH, a hydrogen atom, methyl, ethyl, methoxy, ethoxy, hydroxymethyl, nitrile, or a group CONR⁹₂.

In yet another alternative embodiment, the present invention provides a compound of formula (I) wherein R⁶ is —OH.

In one alternative embodiment, compounds of formula (I) having particular combinations of R⁴ and R⁵, especially when R⁶ is —OH, include those wherein (i) each of R⁴ and R⁵ is optionally substituted monocyclic heteroaryl of 5 or 6 ring atoms such as pyridyl, oxazolyl, thiazolyl, furyl and especially thienyl such a 2-thienyl; (ii) each of R⁴ and R⁵ is optionally substituted phenyl; (iii) one of R⁴ and R⁵ is optionally substituted phenyl and the other is cycloalkyl such as cyclopropyl, cyclobutyl, or especially cyclopentyl or cyclohexyl; and (iv) one of R⁴ and R⁵ is optionally substituted monocyclic heteroaryl of 5 or 6 ring atoms such as pyridyl, thienyl, oxazolyl, thiazolyl, or furyl; and the other is cycloalkyl such as cyclopropyl, cyclobutyl, cyclopentyl or cyclohexyl.

In another alternative embodiment, the present invention provides a compound of formula (I) wherein, R⁴ and R⁵ are both phenyl and R⁶ is —OH.

In yet another alternative embodiment, the present invention provides a compound of formula (I) wherein one of R⁴ and R⁶ is phenyl, the other one of R⁴ and R⁵ is cycloalkyl and R⁶ is —OH.

In a further alternative embodiment, the present invention provides a compound of formula (I) wherein one of R⁴ and R⁵ is phenyl, the other one of R⁴ and R⁵ is cyclohexyl and R⁶ is —OH.

As previously mentioned, it will be appreciated that the carbon atom to which R⁴, R⁵ and R⁶ are attached can be an asymmetric centre so compounds of the invention may be in the form of single enantiomers or mixtures of enantiomers. Examples of configurations of this carbon atom include:

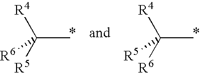

wherein the bond marked * is attached to the ring containing W, V and A.

Further examples of configurations of this carbon atom include:

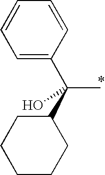

wherein the bond marked * is attached to the ring containing W, V and A.

### The Group R⁷

In one alternative embodiment, the present invention provides a compound of formula (I) wherein R⁷ is an aryl (for example phenyl), aryl-fused-cycloalkyl (for example indanyl) or aryl(C₁-C₈-alkyl)- (for example phenyl-CH₂— or phenyl-CH₂CH₂—) group.

In another alternative embodiment, the present invention provides a compound of formula (I) wherein R⁷ is selected from C₂-C₆-alkenyl (for example 3-methyl-but-2-enyl or allyl), aryl (for example phenyl), heteroaryl (for example thienyl), aryl(C₁-C₈-alkyl)- (for example phenyl-CH₂— or phenyl-CH₂CH₂—), or heteroaryl(C₁-C₈-alkyl)- (for example thienyl-CH₂—)—

In a further alternative embodiment, the present invention provides a compound of formula (I) wherein R⁷ is selected from:
C₁-C₆-alkyl, such as methyl, ethyl, n- or isopropyl, n-, sec- or tertbutyl;
Optionally substituted aryl such as phenyl or naphthyl, or aryl-fused-heterocycloalkyl such as 3,4-methylenedioxyphenyl, 3,4-ethylenedioxyphenyl, or dihydrobenzofuranyl;
Optionally substituted heteroaryl such as pyridyl, pyrrolyl, pyrimidinyl, oxazolyl, isoxazolyl, benzisoxazolyl, benzoxazolyl, thiazolyl, benzothiazolyl, quinolyl, thienyl, benzothienyl, furyl, benzofuryl, imidazolyl, benzimidazolyl, isothiazolyl, benzisothiazolyl, pyrazolyl, isothiazolyl, triazolyl, benzotriazolyl, thiadiazolyl, oxadiazolyl, pyridazinyl, pyridazinyl, triazinyl, indolyl and indazolyl;
Optionally substituted aryl(C₁-C₆-alkyl)—such as those wherein the aryl part is any of the foregoing specifically mentioned aryl groups and the —(C₁-C₆-alkyl)- part is —CH₂— or —CH₂CH₂—;
Optionally substituted aryl-fused-cycloalkyl such as indanyl or 1,2,3,4-tetrahydronaphthalenyl;
Optionally substituted heteroaryl(C₁-C₈-alkyl)—such as those wherein the heteroaryl part is any of the foregoing specifically mentioned heteroaryl groups and the —(C₁-C₆-alkyl)- part is —CH₂— or —CH₂CH₂—; and
Optionally substituted cycloalkyl such as cyclopropyl, cyclobutyl, cyclopentyl or cyclohexyl.

### The Group R⁸

In one alternative embodiment, the present invention provides a compound of formula (I) wherein R⁸ is hydrogen.

### The Groups R¹ and R⁹

In one alternative embodiment, the present invention provides a compound of formula (I) wherein each occurrence of R¹ and R⁹ is independently selected from methyl, ethyl, or a hydrogen atom.

### The Group R¹¹

In one alternative embodiment, the present invention provides a compound of formula (I) wherein R¹¹ is hydrogen or C₁-C₃ alkyl.

In another alternative embodiment, the present invention provides a compound of formula (I) wherein R¹¹ is methyl.

### The Group Y

In one alternative embodiment, the present invention provides a compound of formula (I) wherein Y is an oxygen atom or a group —S(O)ₙ.

In another alternative embodiment, the present invention provides a compound of formula (I) wherein Y is an oxygen atom.

### The Integers n, p, t, u, and v

In one alternative embodiment, the present invention provides a compound of formula (I) wherein n is 1 or 2.

In another alternative embodiment, the present invention provides a compound of formula (I) wherein n is 0.

In one alternative embodiment, the present invention provides a compound of formula (I) wherein p is 1.

In one alternative embodiment, the present invention provides a compound of formula (I) wherein t is 2.

In one alternative embodiment, the present invention provides a compound of formula (I) wherein u is 1 or 2.

In another alternative embodiment, the present invention provides a compound of formula (I) wherein u is 2.

In one alternative embodiment, the present invention provides a compound of formula (I) wherein v is 2.

### The Ring Atoms W, V and A

In one alternative embodiment, particular combinations of W, V and A in the compounds of formula (I) include:

(a) W is a group CR⁸, V is an oxygen atom and A is a nitrogen atom;
(b) W is a group CR⁸, V is a sulfur atom and A is a nitrogen atom;
(c) W is a group CR⁸, V is a nitrogen atom and A is an oxygen atom;
(d) W is a group CR⁸, V is a nitrogen atom and A is a sulfur atom;
(e) W is a nitrogen atom, V is a nitrogen atom and A is an oxygen atom;
(f) W is a nitrogen atom, V is an oxygen atom and A is a nitrogen atom;
(g) W is an oxygen atom, V is a nitrogen atom and A is a nitrogen atom;
(h) W is a nitrogen atom, V is a group CR⁸ and A is an oxygen atom;
(i) W is a nitrogen atom, V is a group CR⁸ and A is a sulphur atom;
(j) W is a group N—R¹¹, V is a group CR⁸ and A is a nitrogen atom;
(k) W is a nitrogen atom, V is an oxygen atom and A is a group CR⁸;
(l) W is a group NR¹¹, V is a nitrogen atom and A is a group CR⁸;
(m) W is an oxygen atom, V is a nitrogen atom and A is a group CR⁸;
(n) W is a nitrogen atom, V is a sulfur atom and A is a nitrogen atom;
(o) W is a nitrogen atom, V is a nitrogen atom and A is a sulfur atom;
(p) W is a sulfur atom, V is a nitrogen atom and A is a nitrogen atom;
(q) W is a nitrogen atom, V is a group CR⁸ and A is a group NR¹¹.

In another alternative embodiment, the present invention provides a compound of formula (I) wherein the 5-membered ring containing W, V and A is selected from:

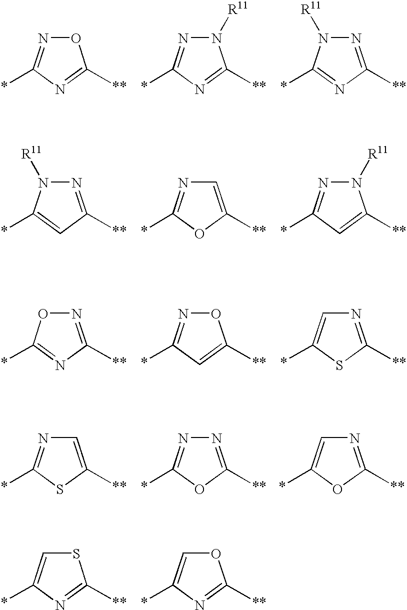

wherein the bond marked * is attached to the group R⁴R⁵R⁶C—, and the bond marked ** is attached to the group —XN⁺; and R¹¹ is as defined herein.

In a further alternative embodiment, the present invention provides a compound of formula (I) wherein the 5-membered ring containing W, V and A is selected from:

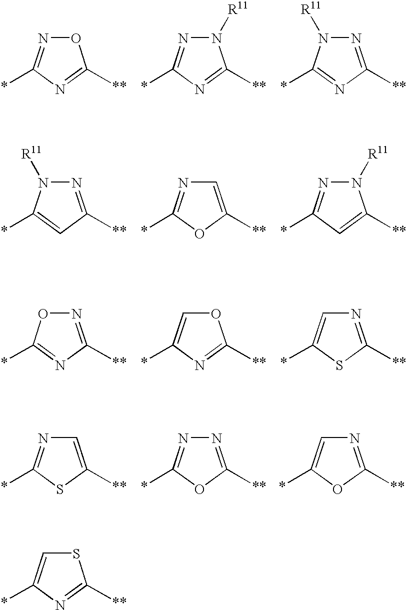

wherein the bond marked * is attached to the group R⁴R⁵R⁶C—, and the bond marked ** is attached to the group —XN⁺; and R¹¹ is as defined herein.

In a yet further alternative embodiment, the present invention provides a compound of formula (I) wherein the 5-membered ring containing W, V and A is selected from:

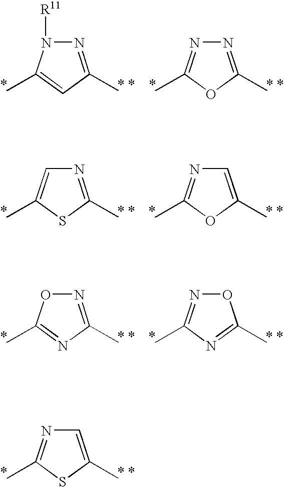

wherein the bond marked * is attached to the group R⁴R⁵R⁶C—, and the bond marked ** is attached to the group —XN⁺; and R¹¹ is as defined herein.

In a yet further still alternative embodiment, the present invention provides a compound of formula (I) wherein the 5-membered ring containing W, V and A is selected from:

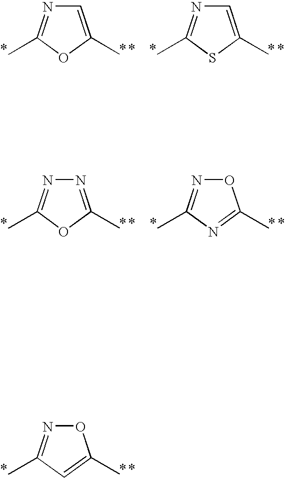

wherein the bond marked * is attached to the group R⁴R⁵R⁶C—, and the bond marked ** is attached to the group —XN⁺.

### The Radical X

In one alternative embodiment, the present invention provides a compound of formula (I)

wherein X is C₁-C₄-alkylene.

In another alternative embodiment, the present invention provides a compound of formula (I) wherein X is C₁-C₃ alkylene.

In a further alternative embodiment, the present invention provides a compound of formula (I) wherein X is ethylene or methylene.

In a yet further alternative embodiment, the present invention provides a compound of formula (I) wherein X is methylene.

### The Radical Z

In one alternative embodiment, the present invention provides a compound of formula (I) wherein Z is C₁-C₄ alkylene (—(CH₂)₁₋₄—) being optionally substituted on up to three carbons in the chain by methyl.

In another alternative embodiment, the present invention provides a compound of formula (I) wherein Z is ethylene or methylene.

### The Azabicyclic Group

In one alternative embodiment, the present invention provides a compound of formula (I) wherein, the group

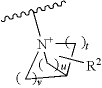

is selected from:

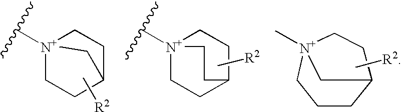

In another alternative embodiment, the present invention provides a compound of formula (I) wherein, the group

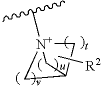

is:

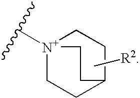

In the compounds of formula (I) defined herein, the group R² may be attached at any carbon atom of the azabicyclic group. In one alternative embodiment, the present invention provides compounds wherein particular attachment points are:

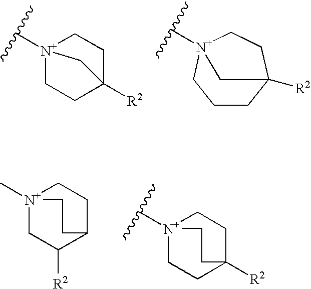

It will be appreciated that certain azabicyclic groups, either inherently or when appropriately substituted by a group R², exist as enantiomeric or diastereomeric groups. All such groups are considered compounds of the invention. Examples of these groups include:

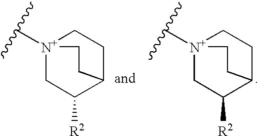

Examples of compounds of the invention include those of the Examples herein.

Particular examples of the compounds of the invention include:
1-[2-(Hydroxy-diphenyl-methyl)-oxazol-5-ylmethyl]-3-phenoxy-1-azoniabicyclo[2.2.2]octane;
(R)-1-[2-((R)—Cyclohexyl-hydroxy-phenyl-methyl)-oxazol-5-ylmethyl]-3-phenoxy-1-azonia-bicyclo[2.2.2]octane;
(R)-3-Benzyloxy-1-[2-((R)-cyclohexyl-hydroxy-phenyl-methyl)-oxazol-5-ylmethyl]-1-azonia-bicyclo[2.2.2]octane;
(S)-3-Benzyloxy-1-[2-((R)-cyclohexyl-hydroxy-phenyl-methyl)-oxazol-5-ylmethyl]-1-azonia-bicyclo[2.2.2]octane;
(S)-1-[2-((R)—Cyclohexyl-hydroxy-phenyl-methyl)-oxazol-5-ylmethyl]-3-phenoxymethyl-1-azonia-bicyclo[2.2.2]octane;
(R)-1-[2-((R)—Cyclohexyl-hydroxy-phenyl-methyl)-oxazol-5-ylmethyl]-3-phenoxymethyl-1-azonia-bicyclo[2.2.2]octane;
(R)-3-Benzyloxy-1-[2-((R)-cyclohexyl-hydroxy-phenyl-methyl)-oxazol-5-ylmethyl]-1-azonia-bicyclo[2.2.2]octane;
(R)-3-Benzyloxy-1-[2-(cyclohexyl-hydroxy-phenyl-methyl)-thiazol-5-ylmethyl]-1-azonia-bicyclo[2.2.2]octane;
(R)-3-Benzyloxy-1-[3-(cyclohexyl-hydroxy-phenyl-methyl)-[1,2,4]oxadiazol-5-ylmethyl]-1-azonia-bicyclo[2.2.2]octane;
(R)-3-Benzyloxy-1-[5-(cyclohexyl-hydroxy-phenyl-methyl)-[1,3,4]oxadiazol-2-ylmethyl]-1-azonia-bicyclo[2.2.2]octane;
(R)-3-Benzyloxy-1-[3-(cyclohexyl-hydroxy-phenyl-methyl)-isoxazol-5-ylmethyl]-1-azonia-bicyclo[2.2.2]octane;
(R)-1-[2-((R)—Cyclohexyl-hydroxy-phenyl-methyl)-oxazol-5-ylmethyl]-3-(4-fluoro-benzyloxy)-1-azonia-bicyclo[2.2.2]octane;
(R)-1-[2-((R)—Cyclohexyl-hydroxy-phenyl-methyl)-oxazol-5-ylmethyl]-3-(4-methyl-benzyloxy)-1-azonia-bicyclo[2.2.2]octane;
(R)-1-[2-((R)—Cyclohexyl-hydroxy-phenyl-methyl)-oxazol-5-ylmethyl]-3-(3-fluoro-benzyloxy)-1-azonia-bicyclo[2.2.2]octane;
1-[2-((R)—Cyclohexyl-hydroxy-phenyl-methyl)-oxazol-5-ylmethyl]-3-phenethyl-1-azonia-bicyclo[2.2.2]octane;
(R)-1-[2-((R)—Cyclohexyl-hydroxy-phenyl-methyl)-oxazol-5-ylmethyl]-3-(3-methyl-but-2-enyloxy)-1-azonia-bicyclo[2.2.2]octane;
(R)-3-Benzylsulfanyl-1-[2-((R)-cyclohexyl-hydroxy-phenyl-methyl)-oxazol-5-ylmethyl]-1-azonia-bicyclo[2.2.2]octane;
(R)-3-Benzyloxy-1-[2-(cyclopentyl-hdroxy-pheyl-methyl)-oxazol-5-ylmethyl]-1-azonia-bicyclo[2.2.2]octane;
(R)-3-(4-Chloro-benzyloxy)-1-[2-(cyclopentyl-hydroxy-phenyl-methyl)-oxazol-5-ylmethyl]-1-azonia-bicyclo[2.2.2]octane;
(R)-1-[2-(Cyclopentyl-hydroxy-phenyl-methyl)-oxazol-5-ylmethyl]-3-(3,4-dichloro-benzyloxy)-1-azonia-bicyclo[2.2.2]octane;
(R)-1-[2-((R)—Cyclohexyl-hydroxy-phenyl-methyl)-oxazol-5-ylmethyl]-3-(thiophen-3-ylmethoxy)-1-azonia-bicyclo[2.2.2]octane;
(R)-3-Benzyloxy-1-[2-(cyclooctyl-hydroxy-phenyl-methyl)-oxazol-5-ylmethyl]-1-azonia-bicyclo[2.2.2]octane;
(R)-3-Benzyloxy-1-[2-(cyclobutyl-hydroxy-phenyl-methyl)-oxazol-5-ylmethyl]-1-azonia-bicyclo[2.2.2]octane;
(R)-3-Allyloxy-1-[2-((R)-cyclohexyl-hydroxy-phenyl-methyl)-oxazol-5-ylmethyl]-1-azonia-bicyclo[2.2.2]octane;
(R)-1-[2-((R)—Cyclohexyl-hydroxy-phenyl-methyl)-oxazol-5-ylmethyl]-3-(2-fluoro-benzyloxy)-1-azonia-bicyclo[2.2.2]octane;
(R)-1-[3-(Cyclohexyl-hydroxy-phenyl-methyl)-isoxazol-5-ylmethyl]-3-(4-fluoro-benzyloxy)-1-azonia-bicyclo[2.2.2]octane;
(R)-3-(3-Chloro-4-methyl-phenoxy)-1-[2-(hydroxyl-diphenyl-methyl)-oxazol-5-ylmethyl]-1-azonia-bicyclo[2.2.2]octane;
(R)-3-(3-Chloro-4-methyl-phenoxy)-1-[2-((R)-cyclohexyl-hydroxy-phenyl-methyl)-oxazol-5-ylmethyl]-1-azonia-bicyclo[2.2.2]octane;
1-[2-((R)—Cyclohexyl-hydroxy-phenyl-methyl)-oxazol-5-ylmethyl]-3-phenylsulfanyl-1-azonia-bicyclo[2.2.2]octane;
(R)-3-(3-Chloro-phenoxy)-1-[2-(hydroxyl-diphenyl-methyl)-oxazol-5-ylmethyl]-1-azonia-bicyclo[2.2.2]octane;
(R)-3-(4-Chloro-phenoxy)-1-[2-((R)-cyclohexyl-hydroxy-phenyl-methyl)-oxazol-5-ylmethyl]-1-azonia-bicyclo[2.2.2]octane;
(R)-3-(3-Chloro-phenoxy)-1-[2-((R)-cyclohexyl-hydroxy-phenyl-methyl)-oxazol-5-ylmethyl]-1-azonia-bicyclo[2.2.2]octane;
(R)-1-[2-((R)—Cyclohexyl-hydroxy-phenyl-methyl)-oxazol-5-ylmethyl]-3-(4-fluoro-phenoxy)-1-azonia-bicyclo[2.2.2]octane;
(R)-1-[2-((R)—Cyclohexyl-hydroxy-phenyl-methyl)-oxazol-5-ylmethyl]-3-(3-fluoro-phenoxy)-1-azonia-bicyclo[2.2.2]octane;
(R)-3-Benzyloxy-1-[5-((R)-cyclohexyl-hydroxy-phenyl-methyl)-[1,3,4]oxadiazol-2-ylmethyl]-1-azonia-bicyclo[2.2.2]octane;
1-[2-((R)—Cyclohexyl-hydroxy-phenyl-methyl)-oxazol-5-ylmethyl]-3-(4-fluoro-phenoxymethyl)-1-azonia-bicyclo[2.2.2]octane;
1-[2-((R)—Cyclohexyl-hydroxy-phenyl-methyl)-oxazol-5-ylmethyl]-3-(3-fluoro-phenoxymethyl)-1-azonia-bicyclo[2.2.2]octane;
1-[2-(Hydroxy-diphenyl-methyl)-oxazol-5-ylmethyl]-3-phenylsulfanyl-1-azonia-bicyclo[2.2.2]octane;
(R)-3-(3-Fluoro-phenoxy)-1-[2-(hydroxy-diphenyl-methyl)-oxazol-5-ylmethyl]-1-azonia-bicyclo[2.2.2]octane;
3-(3-Fluoro-phenoxymethyl)-1-[2-(hydroxy-diphenyl-methyl)-oxazol-5-ylmethyl]-1-azonia-bicyclo[2.2.2]octane;
1-[2-((R)-cyclohexyl-hydroxy-phenyl-methyl)-oxazol-5-ylmethyl]-3-(3-fluoro-phenoxymethyl)-1-azonia-bicyclo[2.2.2]octane;
1-[2-((R)—Cyclohexyl-hydroxy-phenyl-methyl)-oxazol-5-ylmethyl]-3-(3-fluoro-phenoxymethyl)-1-azonia-bicyclo[2.2.2]octane;
1-[2-((R)—Cyclohexyl-hydroxy-phenyl-methyl)-oxazol-5-ylmethyl]-3-(4-fluoro-phenoxymethyl)-1-azonia-bicyclo[2.2.2]octane;
(R)-3-(Benzo[1,3]dioxol-5-yloxy)-1-[2-(hydroxy-diphenyl-methyl)-oxazol-5-ylmethyl]-1-azonia-bicyclo[2.2.2]octane;
1-[3-(Cyclohexyl-hydroxy-phenyl-methyl)-isoxazol-5-ylmethyl]-3-phenylsulfanyl-1-azonia-bicyclo[2.2.2]octane;
1-[5-((R)—Cyclohexyl-hydroxy-phenyl-methyl)-[1,3,4]oxadiazol-2-ylmethyl]-3-phenylsulfanyl-1-azonia-bicyclo[2.2.2]octane;
3-allyloxymethyl-1-[2-((R)-cyclohexyl-hydroxy-phenyl-methyl)-oxazol-5-ylmethyl]-1-azonia-bicyclo[2.2.2]octane;
(S)-1-[2-((R)—Cyclohexyl-hydroxy-phenyl-methyl)-oxazol-5-ylmethyl]-3-phenylsulfanylmethyl-1-azonia-bicyclo[2.2.2]octane;
(R)-3-(4-Fluoro-phenoxy)-1-[3-(hydroxy-diphenyl-methyl)-[1,2,4]oxadiazol-5-ylmethyl]-1-azonia-bicyclo[2.2.2]octane;
(R)-3-(3-Fluoro-phenoxy)-1-[3-(hydroxy-diphenyl-methyl)-[1,2,4]oxadiazol-5-ylmethyl]-1-azonia-bicyclo[2.2.2]octane;
(R)-3-Benzylsulfanyl-1-[2-((R)-cyclohexyl-hydroxy-phenyl-methyl)-oxazol-5-ylmethyl]-1-azonia-bicyclo[2.2.2]octane;
(S)-1-[3-(Cyclohexyl-hydroxy-phenyl-methyl)-isoxazol-5-ylmethyl]-3-phenoxymethyl-1-azonia-bicyclo[2.2.2]octane;
(R)-3-(3-Fluoro-phenylsulfanyl)-1-[2-(hydroxy-diphenyl-methyl)-oxazol-5-ylmethyl]-1-azonia-bicyclo[2.2.2]octane;
(R)-1-[2-((R)—Cyclohexyl-hydroxy-phenyl-methyl)-oxazol-5-ylmethyl]-3-phenylsulfanylmethyl-1-azonia-bicyclo[2.2.2]octane;
(R)-1-[3-(Cyclohexyl-hydroxy-phenyl-methyl)-isoxazol-5-ylmethyl]-3-(3-fluoro-phenoxy)-1-azonia-bicyclo[2.2.2]octane;
(R)-1-[3-(Hydroxy-diphenyl-methyl)-[1,2,4]oxadiazol-5-ylmethyl]-3-phenoxy-1-azonia-bicyclo[2.2.2]octane;
(R)-3-(3-Chloro-4-methyl-phenoxy)-1-[3-(hydroxy-diphenyl-methyl)-[1,2,4]oxadiazol-5-ylmethyl]-1-azonia-bicyclo[2.2.2]octane;
(R)-1-[2-((R)—Cyclohexyl-hydroxy-phenyl-methyl)-oxazol-5-ylmethyl]-3-(3-fluoro-phenylsulfanyl)-1-azonia-bicyclo[2.2.2]octane;
(R)-1-[2-((R)—Cyclohexyl-hydroxy-phenyl-methyl)-oxazol-5-ylmethyl]-3-(3-fluoro-phenylsulfanylmethyl)-1-azonia-bicyclo[2.2.2]octane;
(S)-1-[2-((R)—Cyclohexyl-hydroxy-phenyl-methyl)-oxazol-5-ylmethyl]-3-(4-fluoro-phenylsulfanylmethyl)-1-azonia-bicyclo[2.2.2]octane;
(S)-1-[2-((R)—Cyclohexyl-hydroxy-phenyl-methyl)-oxazol-5-ylmethyl]-3-(3-fluoro-phenylsulfanylmethyl)-1-azonia-bicyclo[2.2.2]octane;
(R)-3-[((E)-But-2-enyl)oxy]-1-[2-((R)-cyclohexyl-hydroxy-phenyl-methyl)-oxazol-5-ylmethyl]-1-azonia-bicyclo[2.2.2]octane;
(R)-1-[3-(Hydroxy-diphenyl-methyl)-[1,2,4]oxadiazol-5-ylmethyl]-3-(thiophen-3-yloxy)-1-azonia-bicyclo[2.2.2]octane;
(R)-3-(3-Chloro-phenoxy)-1-[3-(hydroxy-diphenyl-methyl)-[1,2,4]oxadiazol-5-ylmethyl]-1-azonia-bicyclo[2.2.2]octane;
(R)-1-[3-(Hydroxy-diphenyl-methyl)-[1,2,4]oxadiazol-5-ylmethyl]-3-phenylsulfanyl-1-azonia-bicyclo[2.2.2]octane;
(R)-1-[2-((R)—Cyclohexyl-hydroxy-phenyl-methyl)-oxazol-5-ylmethyl]-3-isobutylsulfanyl-1-azonia-bicyclo[2.2.2]octane;
(S)-1-[2-(Cyclobutyl-hydroxy-phenyl-methyl)-oxazol-5-ylmethyl]-3-phenylsulfanylmethyl-1-azonia-bicyclo[2.2.2]octane;
(R)-1-[3-(Cyclohexyl-hydroxy-phenyl-methyl)-isoxazol-5-ylmethyl]-3-phenylsulfanylmethyl-1-azonia-bicyclo[2.2.2]octane;
(R)-3-Benzylsulfanyl-1-[3-(cyclohexyl-hydroxy-phenyl-methyl)-isoxazol-5-ylmethyl]-1-azonia-bicyclo[2.2.2]octane;
(R)-3-Benzylsulfanyl-1-[3-(cyclohexyl-hydroxy-phenyl-methyl)-[1,2,4]oxadiazol-5-ylmethyl]-1-azonia-bicyclo[2.2.2]octane;
(R)-1-[3-(Cyclohexyl-hydroxy-phenyl-methyl)-[1,2,4]oxadiazol-5-ylmethyl]-3-(3-fluoro-phenoxy)-1-azonia-bicyclo[2.2.2]octane;
(R)-1-[3-(Cyclohexyl-hydroxy-phenyl-methyl)-[1,2,4]oxadiazol-5-ylmethyl]-3-(4-fluoro-benzyloxy)-1-azonia-bicyclo[2.2.2]octane;
(R)-1-[3-(Cyclohexyl-hydroxy-phenyl-methyl)-[1,2,4]oxadiazol-5-ylmethyl]-3-phenylsulfanyl-1-azonia-bicyclo[2.2.2]octane;
(R)-1-[3-(Cyclohexyl-hydroxy-phenyl-methyl)-[1,2,4]oxadiazol-5-ylmethyl]-3-phenylsulfanylmethyl-1-azonia-bicyclo[2.2.2]octane;
(S)-1-[3-(Cyclohexyl-hydroxy-phenyl-methyl)-[1,2,4]oxadiazol-5-ylmethyl]-3-(3-fluoro-phenylsulfanylmethyl)-1-azonia-bicyclo[2.2.2]octane;
(R)-1-[5-((R)—Cyclohexyl-hydroxy-phenyl-methyl)-[1,3,4]oxadiazol-2-ylmethyl]-3-(3-fluoro-phenoxy)-1-azonia-bicyclo[2.2.2]octane;
(R)-3-(Benzo[1,3]dioxol-5-yloxy)-1-[3-(hydroxy-diphenyl-methyl)-[1,2,4]oxadiazol-5-ylmethyl]-1-azonia-bicyclo[2.2.2]octane;
(R)-3-(4-Chloro-phenoxy)-1-[3-(hydroxy-diphenyl-methyl)-[1,2,4]oxadiazol-5-ylmethyl]-1-azonia-bicyclo[2.2.2]octane;
(R)-3-(4-Fluoro-benzyloxy)-1-[3-(hydroxy-diphenyl-methyl)-[1,2,4]oxadiazol-5-ylmethyl]-1-azonia-bicyclo[2.2.2]octane;
(R)-1-[5-((R)—Cyclohexyl-hydroxy-phenyl-methyl)-[1,3,4]oxadiazol-2-ylmethyl]-3-(4-fluoro-phenoxy)-1-azonia-bicyclo[2.2.2]octane;
(R)-1-[3-(Cyclohexyl-hydroxy-phenyl)-methyl)-isoxazol-5-ylmethyl]-3-(4-fluoro-phenoxy)-1-azonia-bicyclo[2.2.2]octane;
   and pharmaceutically acceptable salts thereof.

The present invention is also concerned with pharmaceutical formulations comprising, as an active ingredient, a compound of the invention. Other compounds may be combined with compounds of this invention for the prevention and treatment of inflammatory diseases of the lung. Thus the present invention is also concerned with pharmaceutical compositions for preventing and treating respiratory-tract disorders such as chronic obstructive lung disease, chronic bronchitis, asthma, chronic respiratory obstruction, pulmonary fibrosis, pulmonary emphysema, and allergic rhinitis comprising a therapeutically effective amount of a compound of the invention and one or more other therapeutic agents.

Other compounds may be combined with compounds of this invention for the prevention and treatment of inflammatory diseases of the lung. Accordingly the invention includes a combination of an agent of the invention as hereinbefore described with one or more anti-inflammatory, bronchodilator, antihistamine, decongestant or anti-tussive agents, said agents of the invention hereinbefore described and said combination agents existing in the same or different pharmaceutical compositions, administered separately or simultaneously. Typical combinations would have two or three different pharmaceutical compositions. Suitable therapeutic agents for a combination therapy with compounds of the invention include:

One or more other bronchodilators such as PDE3 inhibitors;
Methyl xanthines such as theophylline;
Other muscarinic receptor antagonists;
A corticosteroid, for example fluticasone propionate, ciclesonide, mometasone furoate or budesonide, or steroids described in WO02/88167, WO02/12266, WO02/100879, WO02/00679, WO03/35668, WO03/48181, WO03/62259, WO03/64445, WO03/72592, WO04/39827 and WO04/66920;
A non-steroidal glucocorticoid receptor agonist;
A β2-adrenoreceptor agonist, for example albuterol (salbutamol), salmeterol, metaproterenol, terbutaline, fenoterol, procaterol, carmoterol, indacaterol, formoterol, arformoterol, picumeterol, GSK-159797, GSK-597901, GSK-159802, GSK-64244, GSK-678007, TA-2005 and also compounds of EP1440966, JP05025045, WO93/18007, WO99/64035, US2002/0055651, US2005/0133417, US2005/5159448, WO00/075114, WO01/42193, WO01/83462, WO02/66422, WO02/70490, WO02/76933, WO03/24439, WO03/42160, WO03/42164, WO03/72539, WO03/91204, WO03/99764, WO04/16578, WO04/016601, WO04/22547, WO04/32921, WO04/33412, WO04/37768, WO04/37773, WO04/37807, WO0439762, WO04/39766, WO04/45618, WO04/46083, WO04/71388, WO04/80964, EP1460064, WO04/087142, WO04/89892, EP01477167, US2004/0242622, US2004/0229904, WO04/108675, WO04/108676, WO05/033121, WO05/040103, WO05/044787, WO04/071388, WO05/058299, WO05/058867, WO05/065650, WO05/066140, WO05/070908, WO05/092840, WO05/092841, WO05/092860, WO05/092887, WO05/092861, WO05/090288, WO05/092087, WO05/080324, WO05/080313, US20050182091, US20050171147, WO05/092870, WO05/077361, DE10258695, WO05/111002, WO05/111005, WO05/110990, US2005/0272769 WO05/110359, WO05/121065, US2006/0019991, WO06/016245, WO06/014704, WO06/031556, WO06/032627, US2006/0106075, US2006/0106213, WO06/051373, WO06/056471;
A leukotriene modulator, for example montelukast, zafirlukast or pranlukast;
protease inhibitors, such as inhibitors of matrix metalloprotease for example MMP12 and TACE inhibitors such as marimastat, DPC-333, GW-3333;
Human neutrophil elastase inhibitors, such as sivelestat and those described in WO04/043942, WO05/021509, WO05/021512, WO05/026123, WO05/026124, WO04/024700, WO04/024701, WO04/020410, WO04/020412, WO05/080372, WO05/082863, WO05/082864, WO03/053930;
Phosphodiesterase-4 (PDE4) inhibitors, for example roflumilast, arofylline, cilomilast, ONO-6126 or IC-485;
Phosphodiesterase-7 inhibitors;
An antitussive agent, such as codeine or dextramorphan;
Kinase inhibitors, particularly P38 MAPKinase inhibitors;
P2X7 antagonists;
iNOS inhibitors;
A non-steroidal anti-inflammatory agent (NSAID), for example ibuprofen or ketoprofen;
A dopamine receptor antagonist;
TNF-α inhibitors, for example anti-TNF monoclonal antibodies, such as Remicade and CDP-870 and TNF receptor immunoglobulin molecules, such as Enbrel;
A2a agonists such as those described in EP1052264 and EP1241176;
A2b antagonists such as those described in WO2002/42298;
Modulators of chemokine receptor function, for example antagonists of CCR1, CCR2, CCR3, CXCR2, CXCR3, CX3CR1 and CCR8, such as SB-332235, SB-656933, SB-265610, SB-225002, MCP-1(9-76), RS-504393, MLN-1202, INCB-3284;
Compounds which modulate the action of prostanoid receptors, for example a PGD₂ (DP1 or CRTH2), or a thromboxane A₂ antagonist e.g. ramatrobant;
Compounds which modulate Th1 or Th2 function, for example, PPAR agonists;
Interleukin 1 receptor antagonists, such as Kineret;
Interleukin 10 agonists, such as Ilodecakin;
HMG-CoA reductase inhibitors (statins); for example rosuvastatin, mevastatin, lovastatin, simvastatin, pravastatin and fluvastatin;
Mucus regulators such as INS-37217, diquafosol, sibenadet, CS-003, talnetant, DNK-333, MSI-1956, gefitinib;
Antiinfective agents (antibiotic or antiviral), and antiallergenic drugs including, but not limited to, anti-histamines.

The weight ratio of the first and second active ingredients may be varied and will depend upon the effective dose of each ingredient. Generally, an effective dose of each will be used.

Any suitable route of administration may be employed for providing a mammal, especially a human, with an effective dosage of a compound of the present invention. In therapeutic use, the active compound may be administered by any convenient, suitable or effective route. Suitable routes of administration are known to those skilled in the art, and include oral, intravenous, rectal, parenteral, topical, ocular, nasal, buccal and pulmonary.

The magnitude of prophylactic or therapeutic dose of a compound of the invention will, of course, vary depending upon a range of factors, including the activity of the specific compound that is used, the age, body weight, diet, general health and sex of the patient, time of administration, the route of administration, the rate of excretion, the use of any other drugs, and the severity of the disease undergoing treatment. In general, the daily dose range for inhalation will lie within the range of from about 0.1 μg to about 10 mg per kg body weight of a human, typically 0.1 μg to about 0.5 mg per kg, and more typically 0.1 μg to 50 μg per kg, in single or divided doses. On the other hand, it may be necessary to use dosages outside these limits in some cases. Compositions suitable for administration by inhalation are known, and may include carriers and/or diluents that are known for use in such compositions. The composition may contain 0.01-99% by weight of active compound. Typically, a unit dose comprises the active compound in an amount of 1 μg to 10 mg. For oral administration suitable doses are 10 μg per kg to 100 mg per kg, typically 40 μg per kg to 4 mg per kg.

Another aspect of the present invention provides pharmaceutical compositions which comprise a compound of the invention and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the invention, additional active ingredient(s), and pharmaceutically acceptable excipients.

The pharmaceutical compositions of the present invention comprise a compound of the invention as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids, and salts of quaternary ammonium compounds with pharmaceutically acceptable counter-ions.

For delivery by inhalation, the active compound is typically in the form of microparticles. They may be prepared by a variety of techniques, including spray-drying, freeze-drying and micronization.

By way of example, a composition of the invention may be prepared as a suspension for delivery from a nebuliser or as an aerosol in a liquid propellant, for example for use in a pressurised metered dose inhaler (PMDI). Propellants suitable for use in a PMDI are known to the skilled person, and include CFC-12, HFA-134a, HFA-227, HCFC-22 (CCl₂F₂) and HFA-152 (C₂H₄F₂) and isobutane.

In a particular embodiment of the invention, a composition of the invention is in dry powder form, for delivery using a dry powder inhaler (DPI). Many types of DPI are known.

Microparticles for delivery by administration may be formulated with excipients that aid delivery and release. For example, in a dry powder formulation, microparticles may be formulated with large carrier particles that aid flow from the DPI into the lung. Suitable carrier particles are known, and include lactose particles; they may have a mass median aerodynamic diameter of greater than 90 μm.

In the case of an aerosol-based formulation, an example is:

| | | |
|---|---|---|
| Compound of the invention | 24 | mg/canister |
| Lecithin, NF Liq. Conc. | 1.2 | mg/canister |
| Trichlorofluoromethane, NF | 4.025 | g/canister |
| Dichlorodifluoromethane, NF | 12.15 | g/canister. |

The active compounds may be dosed as described depending on the inhaler system used. In addition to the active compounds, the administration forms may additionally contain excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321). Additionally, compounds of the invention may be delivered in multi-chamber devices thus allowing for delivery of combination agents.

### Methods of Synthesis

The compounds of the invention of the present invention can be prepared according to the procedures of the following schemes and examples, using appropriate materials, and are further exemplified by the following specific examples. Moreover, by utilising the procedures described with the disclosure contained herein, one of ordinary skill in the art can readily prepare additional compounds of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention may be isolated in the form of their pharmaceutically acceptable salts, such as those described previously herein above. It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 1999, may be used.

Compounds of the invention may be prepared according to the routes illustrated in Schemes 1-11.

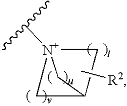

In the schemes that follow, ⁺NR^{c}R^{d}R^{e} represents and R⁴ and R⁵ and D⁻ are as defined above for compounds of formula (I);

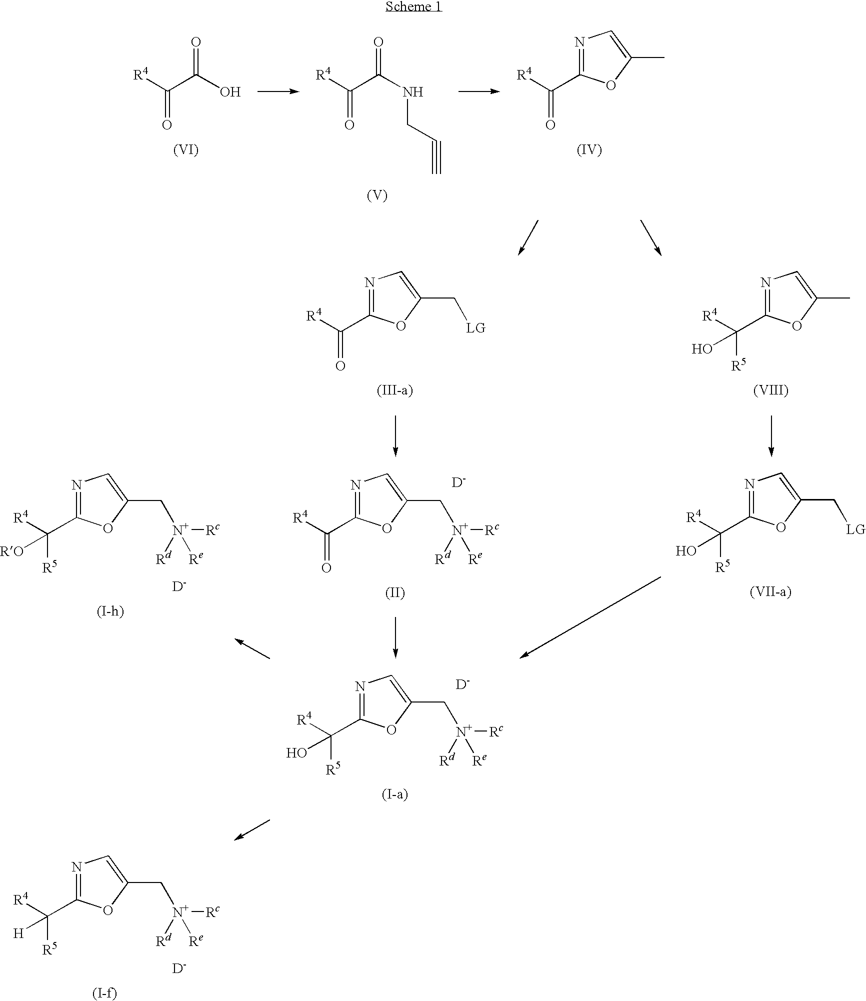

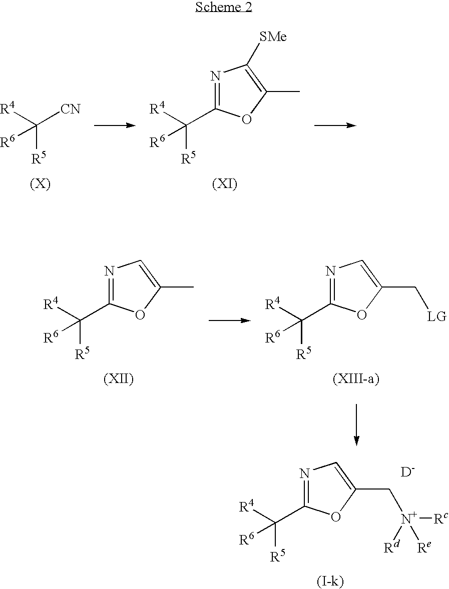

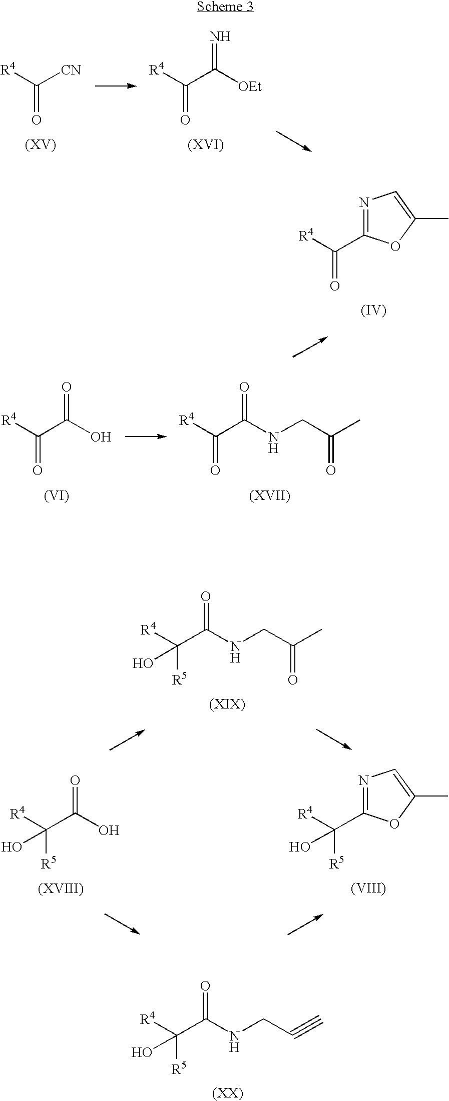

It will be apparent that some compounds can contain a chiral centre and thus exist in enantiomeric forms which can be separated by chiral preparative HPLC techniques using conditions known to those skilled in the art and exemplified below.

Compounds of general formula (I-a), may be prepared from compounds of general formula (II) using methods described below for the preparation of compounds of formula (VIII) from compounds of formula (IV).

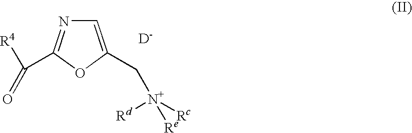

Typically, compounds of formula (Ia) are prepared from compounds of formula (VII-a) as described below.

Compounds of general formula (II) can be prepared from compounds of general formula (III-a):

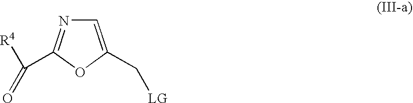

wherein LG represents a leaving group such as bromide, chloride, iodide, by reaction with an amine of formula (XXIII):

R^{c}R^{d}R^{e}N (XXIII)

wherein R^{c}R^{d}R^{e}N represents an azabicyclic compound wherein t, u, v and R² are as described above

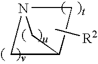

The reaction is performed in a range of solvents, typically a mixture of THF/DCM or acetonitrile/chloroform at a range of temperatures, typically between 0 and the reflux temperature, or more typically, in acetonitrile at a temperature between 0 and 50° C., most typically at 50° C.

Compounds of formula (III-a) wherein LG is bromide can be prepared from compounds of general formula (IV):

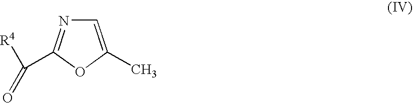

by reaction with a brominating agent such as N-bromosuccinimide in the presence of a radical initiator such as AIBN or benzoyl peroxide. The reaction can be carried out in suitable solvents, such as CCl₄, at a range of temperatures, typically between ambient temperature and the reflux temperature of the solvent.

Compounds of formula (IV) can be prepared from compounds of general formula (V):

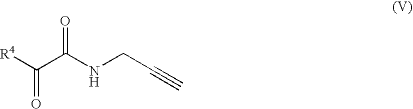

by reaction with an acid such as hydrochloric acid, sulphuric acid, or more typically methanesulfonic or trifluoromethansulfonic acid in a range of solvents such as THF, DCM, water, and typically 1,4-dioxan at a range of temperatures, typically between ambient temperature and the reflux temperature of the solvent.

Alternatively compounds of formula (IV) can be prepared from compounds of general formula (V) by palladium-catalysed cyclisation using a palladium catalyst such as bis(dibenzylideneacetone)palladium in the presence of a ligand such as triphenylphosphine and a base such as sodium tert-butoxide in a solvent such as THF from room temperature to the reflux temperature of the solvent.

Alternatively compounds of formula (IV) can be prepared from compounds of formula (XVI):

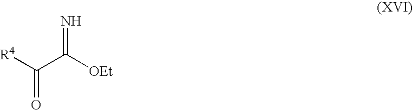

according to the method described in J. Chem. Soc. 1948, 1960. Compounds of general formula (XVI) are known in the art and can be prepared for example from compounds of formula (XV), according to known methods such as those described in Tetrahedron 2002, 58(14), 2813.

Alternatively compounds of formula (IV) can be prepared from compounds of formula (XVII):

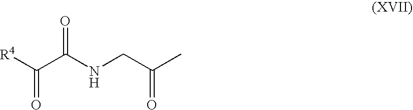

according to the method described in J. Org. Chem., 1938, 2, 319. Compounds of general formula (XVII) are well known in the art and can be prepared by known methods such as those described in GB2214180.

Compounds of general formula (V) can be prepared from compounds of general formula (VI):

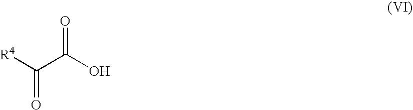

by reaction with propargylamine in the presence of a suitable coupling agent, such as DCC/HOBt or many other known coupling methodologies. Alternatively compounds of formula (VI) may be converted to, for example, the acid chloride and amide formation effected optionally in the presence of a suitable non-nucleophilic base and compatible solvent under well-known conditions. Compounds of general formula (VI) are readily available or can be prepared by known methods.

Alternatively compounds of general formula (I-a) can be prepared from compounds of general formula (VII-a) wherein LG is a leaving group:

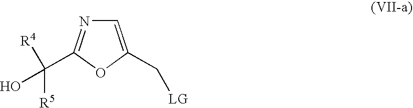

according to methods described above for the preparation of compounds of formula (II) from compounds of formula (III-a)

Compounds of general formula (VII-a) can be prepared from compounds of formula (VIII):

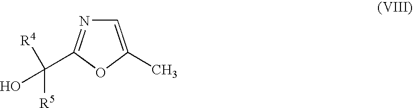

according to methods similar to those used to prepare compounds of formula (III-a) from compounds of formula (IV) as described above.

Compounds of general formula (VIII) can be prepared from compounds of formula (IV) by reaction with a compound of general formula (XXII):

R⁵M (XXII)

wherein M represents a metallic counterion such as Li or MgBr. The reaction may take place in an aprotic organic solvent such as THF or diethyl ether at a range of temperatures, typically between −78° C. and the reflux temperature of the solvent.

Compounds of general formula (XXII) are well known in the art and are readily available or can be prepared by known methods,

Alternatively compounds of formula (VIII) may be prepared from compounds of formula (XIX):

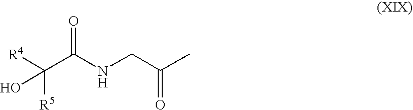

using methods described above for the preparation of compounds of formula (IV) from compounds of formula (XVII). Compounds of general formula (XIX) can be prepared by known methods such as those described in GB2214180.

Alternatively compounds of formula (VIII) may be prepared from compounds of formula (XX):

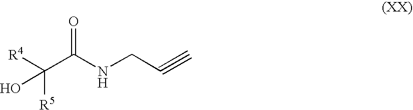

using methods described above for the preparation of compounds of formula (IV) from compounds of formula (V).

Compounds of general formula (XX) can be prepared from compounds of formula (XVIII) using methods described above for the preparation of compounds of formula (V) from compounds of formula (VI).

Compounds of formula (I-f) can be prepared from compounds of formula (I-a) by reaction with a reducing agent such as triethylsilane in the presence of an acid such as trifluoroacetic acid in a solvent such as DCM from room temperature to the reflux temperature of the solvent.

Compounds of formula (I-h) can be prepared from compounds of formula (I-a) by reaction with an alkylating agent of formula (XXIV):

R^{f}LG (XXIV)

wherein R^{f} is C₁-C₆-alkyl and LG is a leaving group such as halogen, tosylate, mesylate. The reaction is performed in the presence of a base such as sodium hydride in a solvent such as THF from 0° C. to the reflux temperature of the solvent.

Compounds of formula (I-k), may be prepared directly from compounds of formula (XIII-a) by reaction with a suitably substituted tertiary amine as described above.

Compounds of general formula (XIII-a) can be prepared from compounds of formula (XII) using methods described above for the preparation of compounds of formula (III-a) from compounds of formula (IV).

Compounds of general formula (XII) may be prepared from compounds of general formula (XI):

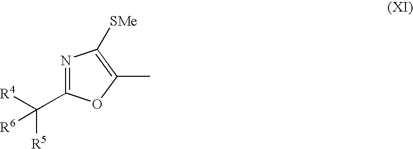

by reaction with a reducing agent such as Raney Nickel in a solvent such as ethanol at a temperature from room temperature to the reflux temperature of the solvent according to the method described in J. Org. Chem. 2006, 71(8), 3026.

Compounds of general formula (XI) may be prepared from compounds of general formula (X):

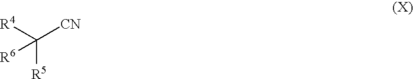

by reaction with 1-(methylthio)acetone in the presence of trifluoromethanesulfonic anhydride in a solvent such as DCM at a temperature from 0° C. to the reflux temperature of the solvent according to the method described in J. Org. Chem. 2006, 71(8), 3026.

Compounds of general formula (X) are well known in the art and can be prepared by known methods, or are commercially available.

Alternatively, compounds of formula (VII-a) may be prepared from compounds of formula (XVIII) as illustrated in Scheme 3a below;

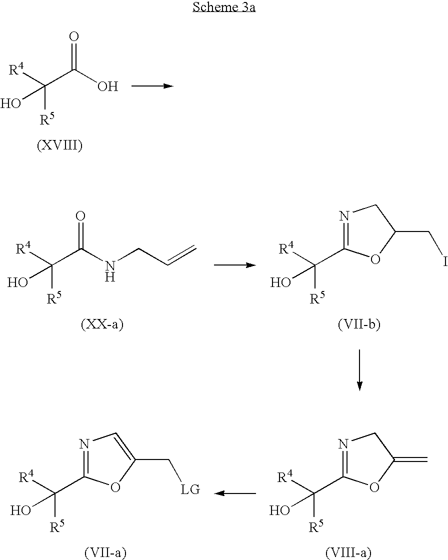

Compounds of formula (VII-a) wherein LG is bromide may be prepared from compounds of formula (VIII-a);

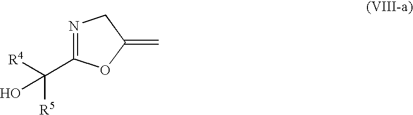

wherein R⁴ and R⁵ are as defined above, by reaction with bromine in a compatible solvent such as carbon tetrachloride, at a temperature of 0° C. to the reflux temperature of the solvent, typically at a temperature between 0 and 25° C.

Compounds of formula (VIII-a) may be prepared from compounds of formula (VII-b);

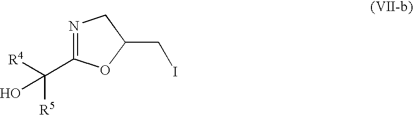

wherein R⁴ and R⁵ are as defined above, by treatment with a non-nucleophilic base such as 1,5-diazabicyclo[4.3.0]non-5-ene (DBN) or 1,8-diazabicyclo[5.4.0]undec-7-ene (DBU) in a compatible solvent, for example toluene, at a temperature from 0-60° C., typically 0-10° C.

Compounds of formula (VII-b) may be prepared from compounds of formula (XX-a);

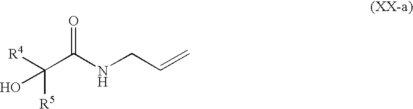

wherein R⁴ and R⁵ are as defined above, by cyclisation in the presence of iodine and a base such as potassium t-butoxide or potassium carbonate in a compatible solvent such as toluene. The reaction is typically conducted at a temperature of 10-30° C.

Compounds of formula (XX-a) may be prepared from compounds of formula (XVIII) using methods analogous to those used in the preparation of compounds of formula (V) from compounds of formula (VI) as described above.

It will be appreciated that compounds of formula (XVIII) in schemes 3 and 3a are chiral when R⁴ and R⁵ are non-identical. When compounds of formula (XVIII) are scalemic (a single enantiomer) then schemes 3 and 3a constitute methods for the preparation of compounds of formula (I-a) that are themselves homochiral. Scalemic examples of compounds of formula (XVIII) are known in the literature, or may be prepared from the racemic form by separation of enantiomers using chiral chromatographic methods, or by separation of diastereomeric salts formed with scalemic bases, or by asymmetric synthesis; see for example US2004192962, WO2000023414, WO9636584, J. Chromatog. (1988), 450(2), 255-269, J. Chem. Soc C (Organic) (1968), 13, 1693-9.

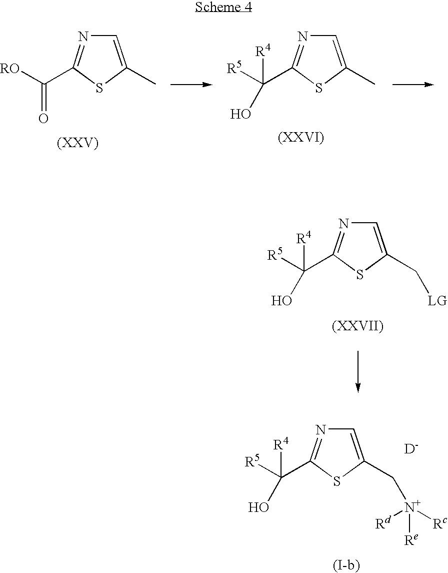

Compounds of Formula (I-b) may be prepared from compounds of Formula (XXVI) by employing a similar sequence of reactions as used to prepare compounds of Formula (I-a) from compounds of Formula (VIII) in Scheme 1 above.

Compounds of formula (XXVI) wherein R⁴ and R⁵ are the same may be prepared from compounds of Formula (XXV) where R is a suitable alkyl group (such as ethyl or methyl) by treatment with an appropriate organometallic reagent such as a Grignard reagent, in a suitable solvent such as THF or diethyl ether. Compounds of Formula (XXVI) wherein R⁴ and R⁵ are dissimilar may be prepared from compounds of Formula (XXV) by converting to an intermediate amide, typically a Weinreb amide, and performing the introduction of R⁴ and R⁵ through their respective organometallic reagents in a stepwise manner.

Compounds of Formula (XXV) are known in the literature—for example, Helv. Chim. Acta 1946, 29, 1957.

More generally, compounds of formula (I-c) wherein W, V and A have the values as illustrated in scheme 5 and R⁴ and R⁵ are as defined above, may be prepared from compounds of formula (XXIX) utilising the sequence of reactions identified above for the preparation of compounds of formula (I-b) from compounds of formula (XXV)

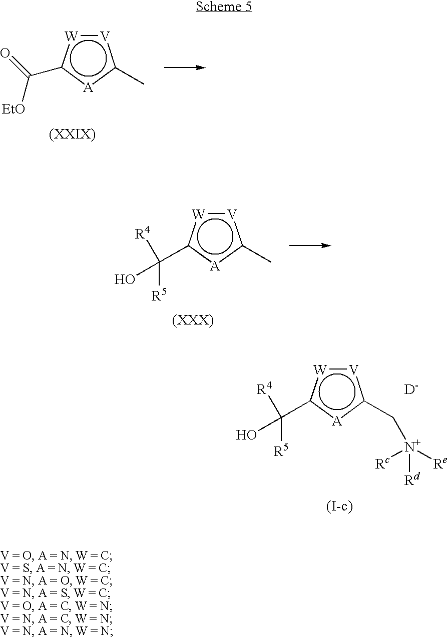

Compounds of formula (XXIX) are commercially available or are known in the literature, for example;

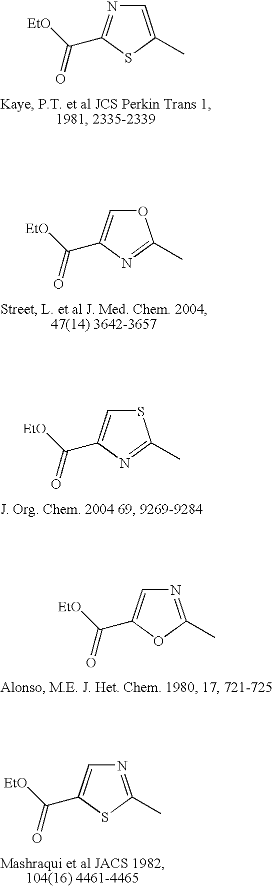

Alternatively, compounds of formula (I-d) may be prepared from compounds of formula (XXVIII) using methods described above for the preparation of compounds of formula (I-a) from compounds of formula (VII-a), Scheme 6;

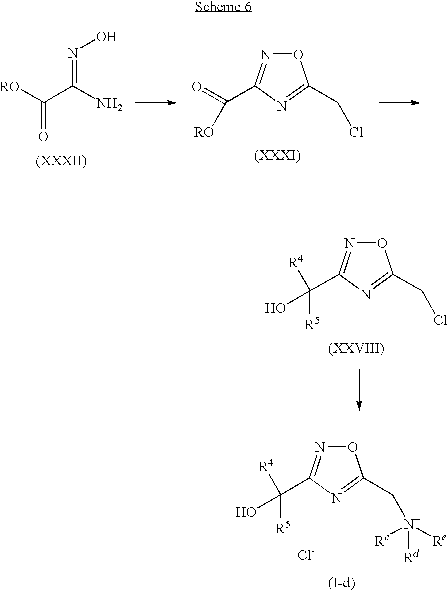

Compounds of formula (XXVIII) may be prepared from compounds of formula (XXXI) using similar methods to those used in the preparation of compounds of formula (XXVI) from compounds of formula (XXV) above.

Compounds of formula (XXXI) where R is typically methyl or ethyl may be prepared from compounds of formula (XXXII) by reaction with chloroacetyl chloride in a solvent such as pyridine or a mixture of chloroform and pyridine at a temperature between 0° C. and ambient temperature. Compounds of formula (XXXII) are commercially available.

Alternatively, Scheme 6a, compounds of formula (XXVIII) may be prepared from compounds of formula (XXXIII) employing methods used for the preparation of compounds of formula (VIII) from compounds of formula (IV) above.

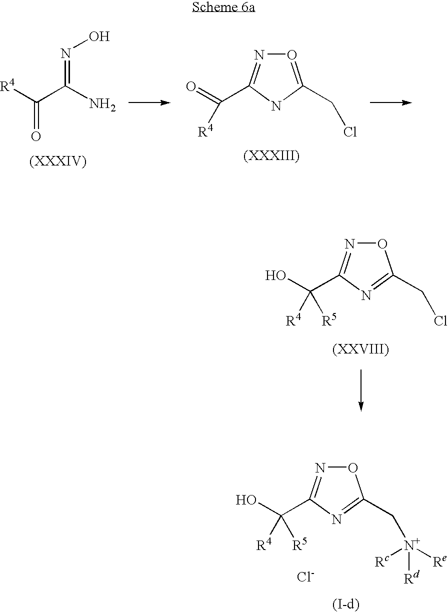

Compounds of formula (XXXIII) may be prepared from compounds of formula (XXXIV) using methods as described for the preparation of compounds of formula (XXXI) from compounds of formula (XXXII) above. Compounds of formula (XXXIV) are known in the art or may be readily prepared according to known methods.

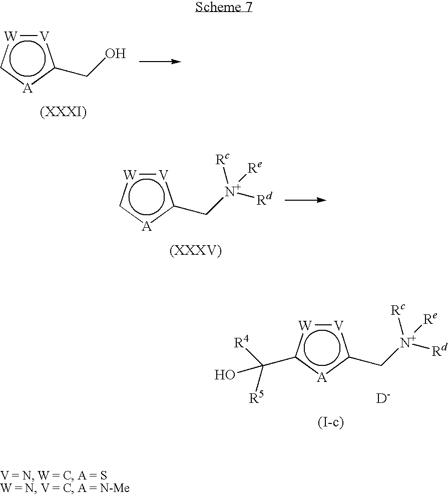

Compounds of formula (I-c) wherein W, V and A have the values as shown in scheme 7, may be prepared from compounds of formula (XXXV) by reaction with a strong base such as n-butyllithium in a compatible solvent such as diethyl ether or tetrahydrofuran followed by reaction with a compound of formula (XXXVI);

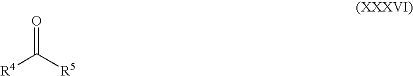

Compounds of formula (XXXVI) are commercially available or readily prepared according to known methods.

Schemes 5, 6 and 7 illustrate how the processes described in schemes 1-4 above may be applied to a variety of different heterocycles. The intermediates of formula (XXXI) are either commercially available or are known compounds.

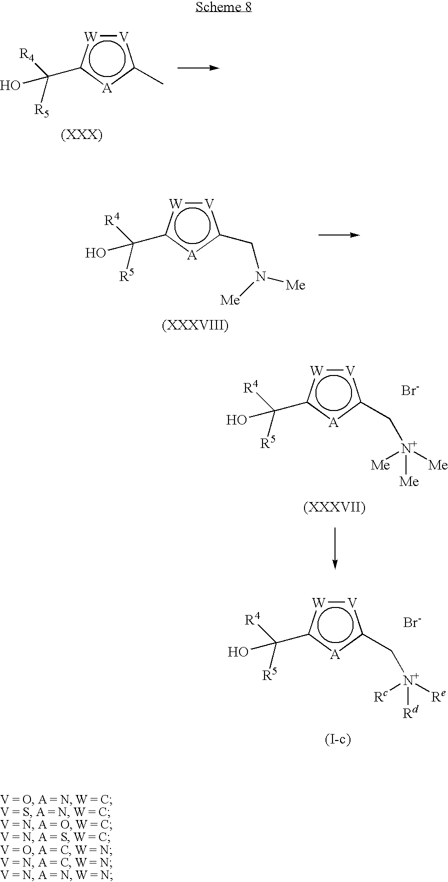

Scheme 8 illustrates an alternative method whereby the ⁺NR^{c}R^{d}R^{e} group can be introduced. Compounds of formula (I-c) may be prepared from compounds of formula (XXXVII) by reaction with a compound of formula (XXIII), in a solvent such as a chloroform/acetonitrile mixture at elevated temperatures, for example at 180° C. in a microwave reactor.

Compounds of formula (XXXVII) can be prepared by the reaction of a compound of formula (XXXVIII) with an alkylating agent, such as MeBr, in an aprotic solvent, such as THF, at elevated temperature.

Compounds of formula (XXXVIII) can by prepared by bromination of a compound of formula (XXX) using a method similar to that described above for the synthesis of compounds of formula (III-a) (LG=bromide). The intermediate bromide is then treated with an alkylamine, such as dimethylamine, in a suitable aprotic solvent, such as THF. at a temperature from 0° C. to ambient temperature.

1,3,4-Oxadiazole (A=oxygen) and thiadiazole (A=sulfur) compounds of formula (I-g) may be prepared as shown in Scheme 9.

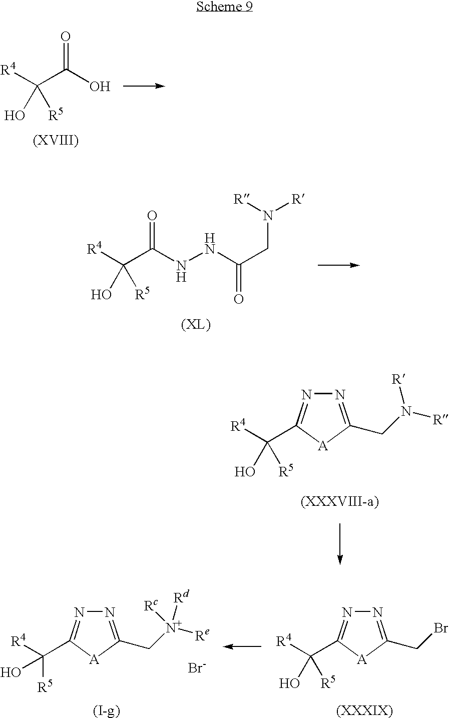

Compounds of formula (I-g) may be prepared from compounds of formula (XXXIX) by methods similar to those used for the preparation of compounds of formula (I-a) from compounds of formula (VII-a).

Compounds of formula (XXXIX) can be prepared from compounds of formula (XXXVIII-a) by treatment with cyanogen bromide (von Braun reaction) in a suitable solvent such as dichloromethane at ambient temperature.

Compounds of formula (XXXVIII-a) where R′ and R″ are lower alkyl, and where A=oxygen may be prepared from compounds of formula (XL) by treatment with acetic anhydride at elevated temperature, for example at 90° C. and where A=sulfur by treatment with Lawesson's reagent in an inert solvent such as toluene, at elevated temperature, typically reflux temperature.

Compounds of formula (XL) may be prepared from compounds of formula (XVIII) by reaction with compounds of formula (XLI) in the presence of a suitable coupling agent such as carbonyl diimidazole, in a suitable solvent, for example dichloromethane at ambient temperature. Compounds of formula (XLI) are known in the art or are commercially available.

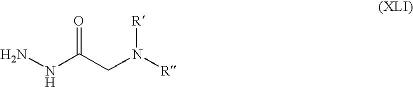

Compounds of formula (I-i) may be prepared (Scheme 10) from compounds of formula (XLII) wherein PG is a protecting group such as a tetrahydropyranyl group, by a) removal of the protecting group by appropriate means, such as under acidic conditions, followed by b) conversion of the liberated alcohol group to a leaving group, such as bromide by treatment with CBr₄ and triphenyl phosphine in a solvent such as dichloromethane at ambient temperature followed by c) reaction with a suitable tertiary amine NR^{c}R^{d}R^{e} employing conditions similar to those described for the preparation of compounds of formula (I-a) from compounds of formula (VII-a).

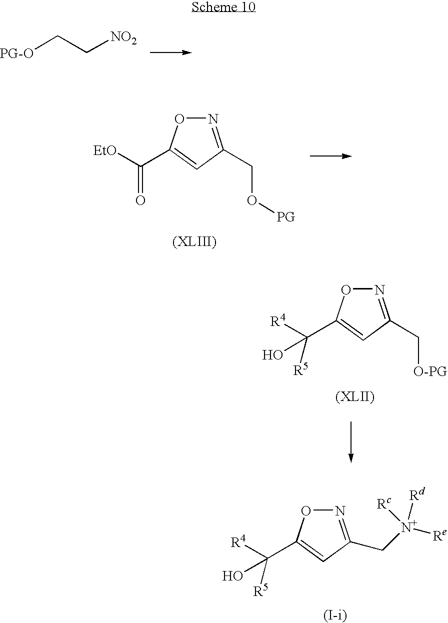

Compounds of formula (XLII) can be prepared from compounds of formula (XLIII) using methods similar to those described above for the conversion of compounds of formula (XXV) to those of formula (XXVI).

Compounds of formula (XLIII) can be prepared from a suitably protected nitro alcohol by reaction with ethyl propiolate and t-BOC anhydride in a suitable solvent such as acetonitrile, optionally in the presence of a catalyst such as 4-dimethylaminopyridine (DMAP) at ambient temperature (See Tetrahedron 2003, 59, 5437-5440)

Isoxazoles of formula (I-j) may be prepared according to the methods outlined in Scheme 11;

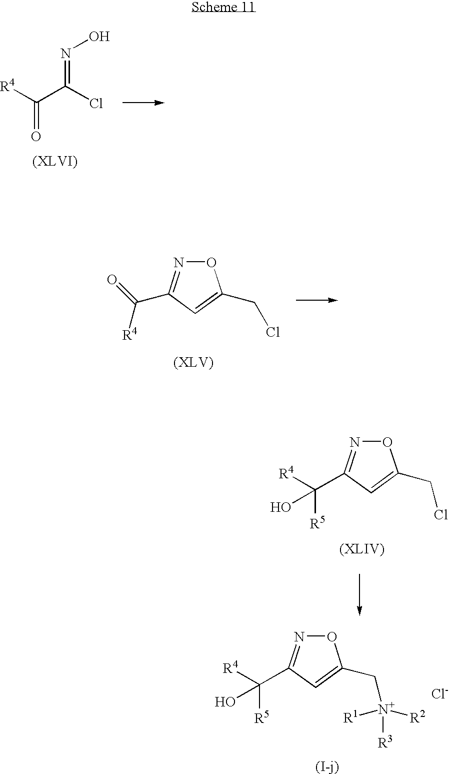

Compounds of formula (I-j) may be prepared from compounds of formula (XLV) using a similar set of procedures as described for the preparation of compounds of formula (I-d) from compounds of formula (XXXIII). Compounds of formula (XLV) and their preparation from compounds of formula (XLVI) are described in the literature, Medicinal Chemistry Research 2001, 10(9), 615-633

Compounds of formula (XXIII) are generally known in the literature, or may be prepared according to methods described in the literature.

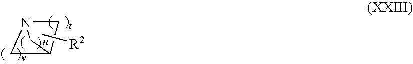

More specifically;

compounds of formula (XXIII) wherein t, u and v are each 2 and R² represents the group —O-aryl or —S-aryl are described in EP497415 and EP458214;

compounds of formula (XXIII) wherein t, u and v are each 2 and R² represents the groups —Y—R⁷ or —Z—Y—R⁷, wherein Z, Y and R⁷ are as described above for compounds of formula (I), are described in EP458214, Bioorg Med Chem Lett 2004, 3781-3784;

compounds of formula (XXIII) wherein t, u and v are 2 and R² represents the group —(Z)ₚ—R⁷ as described above for compounds of formula (I), are described for example in Bioorg Med Chem 2003, 1493-1502; Bioorg Med Chem 2002, 2681-2691; Biorg Med Chem 2000, 449-454; J Med Chem 1997, 538-546; J Med Chem 1995, 3469-3481; J Med Chem 1992, 1280-1290; J Med Chem 1992, 295-305; J Med Chem 1990, 2052-2059 and J Chem Soc Chem Commun, 1988, 1618-1619;

compounds of formula (XXIII) wherein R² represents the group —Y—R⁷ and Y represents —N(H)C(O)-are described in WO04/052461 and J Med Chem 1993, 683-9;

compounds of formula (XXIII) wherein t=1, u=1, v=3 and R² represents —(Z)ₚ—R⁷ are described in EP413545;

and

compounds of formula (XXIII) may be prepared according to methods described above.

The following non-limiting Examples illustrate the invention.

### General Experimental Details:

All reactions were carried out under an atmosphere of nitrogen unless specified otherwise.

NMR spectra were obtained on a Varian Unity Inova 400 spectrometer with a 5 mm inverse detection triple resonance probe operating at 400 MHz or on a Bruker Advance DRX 400 spectrometer with a 5 mm inverse detection triple resonance TXI probe operating at 400 MHz or on a Bruker Avance DPX 300 spectrometer with a standard 5 mm dual frequency probe operating at 300 MHz. Shifts are given in ppm relative to tetramethylsilane.

Where products were purified by column chromatography, ‘flash silica’ refers to silica gel for chromatography, 0.035 to 0.070 mm (220 to 440 mesh) (e.g. Fluka silica gel 60), and an applied pressure of nitrogen up to 10 p.s.i accelerated column elution or use of the CombiFlash® Companion purification system. Where thin layer chromatography (TLC) has been used, it refers to silica gel TLC using plates, typically 3×6 cm silica gel on aluminum foil plates with a fluorescent indicator (254 nm), (e.g. Fluka 60778). All solvents and commercial reagents were used as received.

The Liquid Chromatography Mass Spectroscopy (LC/MS) and chiral preparative HPLC systems used:

### Method 1

Micromass Platform LCT with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow mL/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection - MS, ELS, UV (100 μl split to MS with in-line UV detector) MS ionisation
method - Electrospray (positive ion)

### Method 2

Waters Micromass ZQ with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection - MS, ELS, UV (100 μl split to MS with in-line UV detector) MS ionisation
method - Electrospray (positive and negative ion)

### Method 3

Chiral compounds were separated into pure enantiomers using a 250×20 mm Chiralpak® IA column packed with amylase tris(3,5-dimethylphenylcarbamate) immobilized on 5 μm silica gel. The column was eluted with desired solvents, buffered with 0.1% diethylamine. Flow rate, 18 mL/min. Wavelength, 220 nm.

### Method 4

Chiral compounds were separated into pure enantiomers using a 25×0.46 cm Chiralpak® IA column packed with amylase tris(3,5-dimethylphenylcarbamate) immobilized on 5 μm silica gel. The column was eluted with desired solvents. Flow rate, 1 mL/min. Wavelength, 220 nm.

### Method 5

Chiral compounds were separated into pure enantiomers using a 250×4.6 mm Chiralpak® AD column packed with amylase tris(3,5-dimethylphenylcarbamate) immobilized on 10 μm silica gel. The column was eluted with desired solvents, buffered with 0.1% diethylamine. Flow rate, 8 mL/min. Wavelength, 220 nm.

### Method 6

Waters Micromass ZQ2000 with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow mL/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection - MS, ELS, UV (100 μl split to MS with in-line UV detector) MS ionisation
method - Electrospray (positive ion)

### Method 7

Waters Platform LC Quadrupole mass spectrometer with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection - MS, ELS, UV (200 μl split to MS with in-line UV detector) MS ionisation
method - Electrospray (positive and negative ion).

### Method 8

Waters ZMD mass spectrometer with a C18-reverse-phase column (30×4.6 mm Phenomenex Luna 3 μm particle size), elution with A: water+0.1% formic acid; B: acetonitrile+0.1% formic acid. Gradient:

| Gradient-Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection - MS, ELS, UV (200 μl split to MS with in-line UV detector) MS ionisation
method - Electrospray (positive and negative ion).

### Method 9

Chiral compounds were separated into pure enantiomers using a 50×250 Varian ‘Load and Lock’ column packed with Chiralpak OJ (20 um) silica. The column was eluted with 80:20 isohexanes:ethanol at 118 mL/min flow rate monitored at 220 nm.

Abbreviations used in the experimental section: AIBN=2,2′-azobis(2-methylpropionitrile); DCM=dichloromethane; DEA=diethylamine; DIPEA=diisopropylethylamine; DMAP=4-dimethylaminopyridine; DMF=dimethylformamide; DMSO=dimethyl sulfoxide; EtOAc=ethyl acetate; EtOH=ethanol; IMS=industrial methylated spirit; IPA=2-propanol; MeOH=methanol; RT=room temperature; Rt=retention time; TFA=trifluoroacetic acid; THF=tetrahydrofuran; Sat=saturated; MeCN=acetonitrile; SCX=strong cation exchange chromatography.

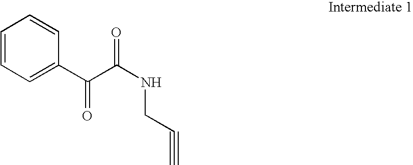

### 2-Oxo-2-phenyl-N-prop-2-ynyl-acetamide

Oxalyl chloride (6.1 g, 48 mmol) was added to a solution of phenylglyoxylic acid (6.0 g, 40 mmol) and 3 drops of DMF in dry DCM (50 mL). The reaction mixture was stirred at room temperature for 3 hours then the solvent was removed. The residue was taken up in dry DCM (50 mL) and the solution was cooled to 0° C. A mixture of propargyl amine (2.2 g, 40 mmol) and triethylamine (4.05 g, 40 mmol) was added cautiously over a period of 10 minutes then the mixture was allowed to warm to room temperature. Stirring was continued for 2.5 hours then water (10 mL) was added. The mixture was washed with 1M HCl (2×20 mL), sat. NaHCO₃ (aq.) (2×20 mL) then brine. The organic phase was dried (Na₂SO₄), concentrated and the residue crystallized from cyclohexane to afford the title compound as a light brown solid (5.75 g, 76%). LCMS (Method 2): Rt 2.47 min, m/z 188 [MH⁺].

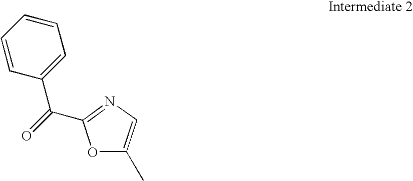

### (5-Methyl-oxazol-2-yl)-phenyl-methanone

Methane sulphonic acid (10 g, 104 mmol) was added dropwise to a solution of 2-oxo-2-phenyl-N-prop-2-ynyl-acetamide (2.4 g, 12.83 mmol) in 1,4-dioxane (20 mL). The resulting solution was heated at 90° C. for 66 hours. The reaction mixture was cooled and the solvent was removed. The dark residue was partitioned between DCM and water. The DCM fraction was washed with 1M HCl (2×), sat. NaHCO₃ (2×), brine and dried (Na₂SO₄). Purification by column chromatography using 4:1 cyclohexane/ethyl acetate as eluent gave the title compound as an off white solid (1.0 g, 41%) LCMS (Method 2): Rt 2.94 min, m/z 188 [MH⁺].

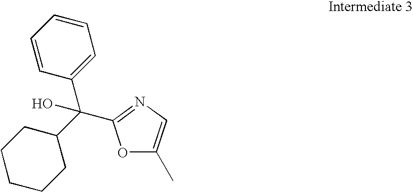

### (+/−)-Cyclohexyl-(5-methyl-oxazol-2-yl)-phenyl-methanol

A solution of (5-methyl-oxazol-2-yl)-phenyl-methanone (3.0 g, 16 mmol) in 32 mL dry THF at 0° C. under nitrogen was treated dropwise over 10 minutes with a 2M solution of cyclohexylmagnesium chloride in diethyl ether (10 mL, 20 mmol). The resulting deep yellow solution was stirred at 0° C. for 30 mins during which time a precipitate formed, and then at room temperature for 1.5 hours. The reaction mixture was cooled to 0° C. again and treated cautiously with sat. NH₄Cl (10 mL). The mixture was stirred at room temperature for 10 mins then diluted with water (10 mL). The phases were separated and the organic phase was washed with brine. The combined aqueous phase was extracted with DCM (3×20 mL) and the combined organic phase was dried (MgSO₄) and concentrated in vacuo to give the crude product which was triturated with ether (10 mL), filtered off and dried to give the title compound (3.65 g, 84%). LCMS (Method 2): Rt 3.78 min, m/z 272 [MH⁺]

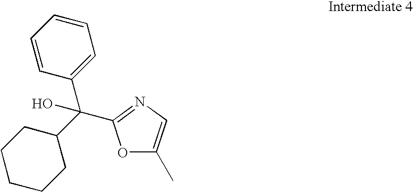

### Cyclohexyl-(5-methyl-oxazol-2-yl)-phenyl-methanol, enantiomer 1

(+/−)-Cyclohexyl-(5-methyl-oxazol-2-yl)-phenyl-methanol was separated into individual enantiomers using method 4 and heptane/IPA/acetonitrile (98.5:1.0:0.5) as eluent. Rt 8.84 min.

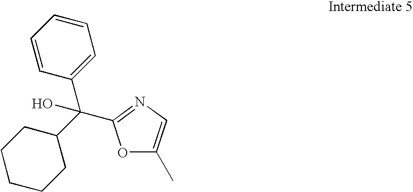

### Cyclohexyl-(5-methyl-oxazol-2-yl)-phenyl-methanol, enantiomer 2

(+/−)-Cyclohexyl-(5-methyl-oxazol-2-yl)-phenyl-methanol was separated into individual enantiomers using method 4 and heptane/IPA/acetonitrile (98.5:1.0:0.5) as eluent. Rt 10.31 min.

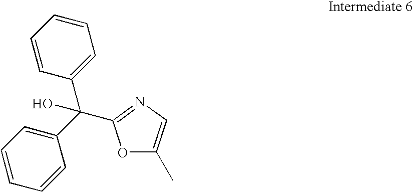

### (5-Methyl-oxazol-2-yl)-diphenyl-methanol

The title compound was prepared from (5-methyl-oxazol-2-yl)-phenyl-methanone using phenylmagnesium bromide under similar conditions to those described for cyclohexyl-(5-methyl-oxazol-2-yl)-phenyl-methanol (2.06 g, 73%). LCMS (Method 7): Rt 3.78 min 266 [MH⁺]

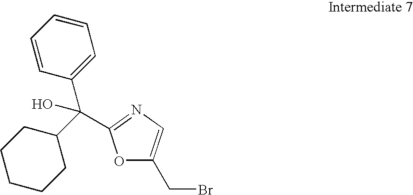

### (5-Bromomethyl-oxazol-2-yl)-cyclohexyl-phenyl-methanol

A solution of cyclohexyl-(5-methyl-oxazol-2-yl)-phenyl-methanol (3.0 g, 11.1 mmol) in 1,2-dichloroethane (22 mL) was treated with N-bromo-succinimide (2.16 g, 12.2 mmol) followed by 2,2′-azobis(2-methylpropionitrile) (0.18 g, 2.1 mmol). The mixture was heated to 80° C. for 2.5 h and then allowed to cool to room temperature. Sat. NaHCO₃ solution was added and the phases were separated. The organic layer was washed with brine and the combined aqueous layers were extracted with DCM. The combined organic phase was dried (MgSO₄) and concentrated in vacuo to give the crude product as a brown oil. Purification using column chromatography using 33-100% DCM/cyclohexane followed by 25% EtOAc/DCM as eluent gave the title compound (1.85 g, 48%). LCMS (Method 2): Rt 4.27 min, m/z 350, 352 [MH⁺]

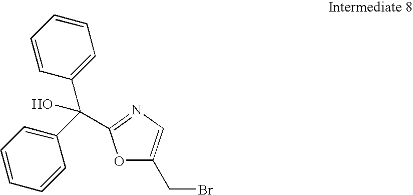

### (5-Bromomethyl-oxazol-2-yl)-diphenyl-methanol

The title compound was prepared from (5-methyl-oxazol-2-yl)-diphenyl-methanol under similar conditions to those described for (5-bromomethyl-oxazol-2-yl)-cyclohexyl-phenyl-methanol (1.63 g, 63%). LCMS (Method 2): Rt 3.53 m/z 326, 328 [MH⁺—H₂O]

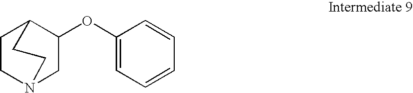

### 3-Phenoxy-1-aza-bicyclo[2.2.2]octane, enantiomer 1

The enantiomers of the title compound were separated by chiral separation method 5 employing ethanol as the eluent. Enantiomer 1: Rₜ 12.50 min. ¹H NMR, 400 MHz, CDCl₃: δ 7.25-7.30 (2H, m), 6.90-6.95 (1H, m), 6.82-6.90 (2H, m), 4.35-4.40 (1H, m), 3.22-3.31 (1H, m), 2.93-3.05 (1H, m), 2.73-2.92 (4H, m), 1.95-2.20 (2H, m), 1.70-1.80 (1H, m), 1.51-1.61 (1H, m), 1.34-1.45 (1H, m).

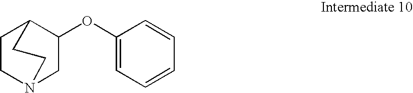

### 3-Phenoxy-1-aza-bicyclo[2.2.2]octane, enantiomer 2

The enantiomers of the title compound were separated by chiral separation method 5 using ethanol as the eluent. Enantiomer 2: Rₜ 17.00 min. ¹H NMR, 400 MHz, CDCl₃: δ 7.25-7.30 (2H, m), 6.90-6.95 (1H, m), 6.82-6.90 (2H, m), 4.35-4.40 (1H, m), 3.22-3.31 (1H, m), 2.93-3.05 (1H, m), 2.73-2.92 (4H, m), 1.95-2.20 (2H, m), 1.70-1.80 (1H, m), 1.51-1.61 (1H, m), 1.34-1.45 (1H, m).

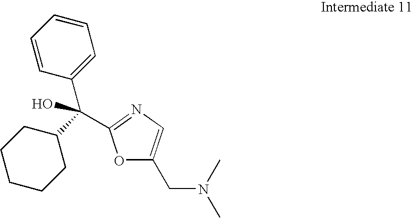

### (S) Cyclohexyl-(5-dimethylaminomethyl-oxazol-2-yl)-phenyl-methanol

A solution of (5-bromomethyl-oxazol-2-yl)-cyclohexyl-phenyl-methanol (3.2 g, 9.2 mmol) in 40 mL THF was treated with a 2M solution of dimethylamine in THF (40 mL, 80 mmol). A suspension formed after stirring for a few minutes. The reaction mixture stood at room temperature overnight and then the solid was filtered off and discarded. The filtrate was concentrated under reduced pressure and the residue was partitioned between DCM and sat. NaHCO₃ solution. The organic layer was dried (Na₂SO₄) and evaporated to give cyclohexyl-(5-dimethylaminomethyl-oxazol-2-yl)-phenyl-methanol as a solid (2.74 g, 95%). LCMS (Method 1): Rt 6.57 min, m/z 315 [MH⁺] ¹H NMR (400 MHz, DMSO-d₆): δ 7.40-7.46 (m, 2H), 7.27-7.34 (m, 2H), 7.18-7.22 (m, 1H), 6.98 (s, 1H), 5.90 (s, 1H), 3.45 (s, 2H), 2.22 (m, 1H), 2.10 (s, 6H), 1.42-1.74 (m, 4H), 0.92-1.29 (m, 6H).

The two enantiomers of cyclohexyl-(5-dimethylaminomethyl-oxazol-2-yl)-phenyl-methanol (2.74 g) were separated by chiral preparative HPLC method 3 with 5% ethanol in heptane at 15 mL/min. The first eluting enantiomer (Rt 8.5 min), gave the title compound as a white solid (0.73 g, 27%). LCMS (Method 1): Rt 6.50 min, m/z 315 [MH⁺]. ¹H NMR (300 MHz, CDCl3): δ 7.64 (d, 2H), 7.33 (t, 2H), 7.24 (t, 1H), 6.84 (s, 1H), 3.70 (br.s, 1H), 3.54 (dd_{AB}, 2H), 2.29-2.32 (m, 1H), 2.25 (s, 6H), 1.62-1.76 (m, 3H), 1.12-1.39 (m, 7H).

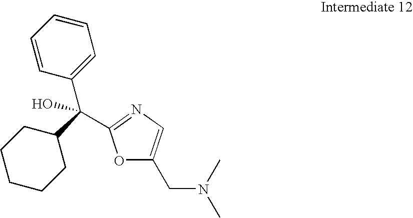

### (R) Cyclohexyl-(5-dimethylaminomethyl-oxazol-2-yl)-phenyl-methanol

The second eluting enantiomer (Rt 10.3 min), from the procedure described under intermediate 11 gave the title compound as a white solid (1.04 g, 38%). LCMS (Method 1): Rt 6.48 min, m/z 315 [MH⁺]. ¹H NMR (CDCl₃): δ¹H NMR (300 MHz, CDCl₃): δ 7.64 (d, 2H), 7.33 (t, 2H), 7.24 (t, 1H), 6.84 (s, 1H), 3.70 (br.s, 1H), 3.54 (dd_{AB}, 2H), 2.29-2.35 (m, 1H), 2.25 (s, 6H), 1.62-1.76 (m, 3H), 1.10-1.39 (m, 7H).

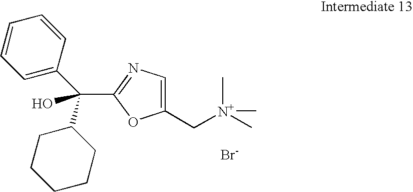

### [2-((S)—Cyclohexyl-hydroxy-phenyl-methyl)-oxazol-5-ylmethyl]-trimethyl-ammonium bromide

A reaction vessel was charged with (S)-cyclohexyl-(5-dimethylaminomethyl-oxazol-2-yl)-phenyl-methanol (100 mg, 0.318 mmol), 1 mL methyl bromide in THF (30% w/v, 300 mg, 3.18 mmol), and chloroform (1 mL). The reaction was stirred and heated at 50° C. for 24 h. The reaction was allowed to cool to room temperature and the solvent removed in vacuo. The crude residue was washed with chloroform (2 mL). The solid was filtered and dried in vacuo to afford the title compound in quantitative yield (129.9 mg, 100%). LCMS (Method 2): Rt 4.24 min, m/z 329 [M⁺]. ¹H NMR (CDCl₃): δ 0.95-1.68 (m, 10H), 2.22-2.32 (m, 1H), 3.02 (bs, 9H), 4.43 (s, 1H), 4.54-4.65 (m, 2H), 7.16-7.21 (m, 1H), 7.24-7.29 (m, 2H), 7.36-7.37 (m, 1H), 7.46-7.49 (m, 2H).

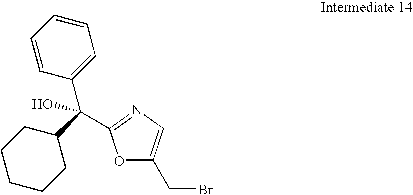

### (R)-(5-Bromomethyl-oxazol-2-yl)-cyclohexyl-phenyl-methanol

A solution of (R)-cyclohexyl-(5-dimethylaminomethyl-oxazol-2-yl)-phenyl-methanol (Intermediate 12) (500 mg, 1.6 mmol) in DCM (8 mL) was added to a solution of cyanogen bromide (3 M in DCM, 1.06 mL) over 10 min at room temperature. After a further 35 min, the reaction was evaporated in vacuo and purified by silica gel chromatography (eluting with petroleum ether and DCM) to give the title compound (439 mg, 77%) as a white solid. ¹H NMR (300 MHz, CDCl₃): δ 7.66-7.61 (2H, m), 7.38-7.32 (2H, m), 7.29-7.23 (1H, m), 6.97 (1H, s), 4.47 (2H, s), 3.65 (1H, s), 2.35-2.26 (1H, m), 1.78-1.61 (3H, m), 1.39-1.11 (7H, m).

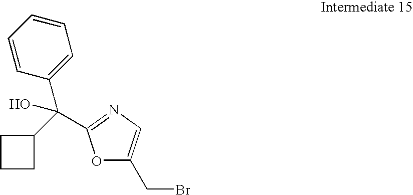

### (5-Bromomethyl-oxazol-2-yl)-cyclobutyl-phenyl-methanol, Enantiomer 2

Step 1. (RS)—Cyclobutyl-(5-dimethylaminomethyl-oxazol-2-yl)-phenyl-methanol was obtained from Intermediate 2 by methods analogous to those used in the preparation of Intermediate 11. The compound was resolved into its two enantiomers by means of chiral HPLC (Method 3, eluting with 90:10:0.1 heptane/IPA/DEA). Retention time of Enantiomer 1 was 7.41 min and retention time of Enantiomer 2 was 9.00 min. LCMS for Enantiomer 2 (Method 7, 1.95 min) MH⁺=287.

Step 2. The title compound (73 mg, 75%) was obtained from the foregoing compound, Enantiomer 2 by application of the procedure described for Intermediate 14. Data for (5-bromomethyl-oxazol-2-yl)-cyclobutyl-phenyl-methanol, Enantiomer 2: LCMS (Method 7, 3.75 min) MH⁺=324.

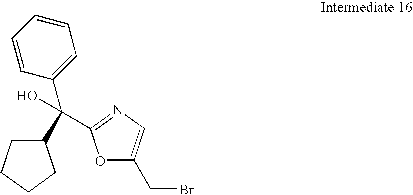

### (R)-(5-Bromomethyl-oxazol-2-yl)-cyclopentyl-phenyl-methanol

The title compound (268 mg, 52%) was obtained from (R)—Cyclopentyl-(5-dimethylaminomethyl-oxazol-2-yl)-phenyl-methanol (described as Example 19 in WO 2007/017669) by application of the procedure described for Intermediate 14. Data for (R)-(5-bromomethyl-oxazol-2-yl)-cyclopentyl-phenyl-methanol: LCMS (Method 7, 4.00 min). MH⁺=336.05.

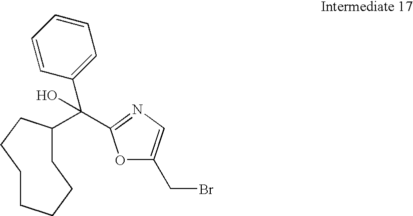

### (5-Bromomethyl-oxazol-2-yl)-cyclooctyl-phenyl-methanol, Enantiomer 2

Step 1. (RS)—Cyclooctyl-(5-dimethylaminomethyl-oxazol-2-yl)-phenyl-methanol was obtained from Intermediate 2 by methods analogous to those used in the preparation of Intermediate 12. The compound was resolved into its two enantiomers by means of chiral HPLC (Method 3, eluting with 97.4/2.5/0.1 heptane/IPA/DEA). Retention time of Enantiomer 1 was 17.47 min and retention time of Enantiomer 2 was 19.54 min. LCMS (Method 8, 2.35 min). MH⁺=343.

Step 2. The title compound (159 mg, 65%) was obtained from the foregoing compound, Enantiomer 2 by application of the procedure described for Intermediate 14. Data for (5-bromomethyl-oxazol-2-yl)-cyclooctyl-phenyl-methanol, Enantiomer 2: LCMS (Method 8, 4.49 min). MH⁺=378.

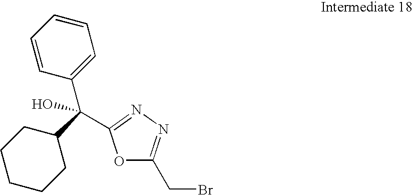

### (R)-(5-Bromomethyl-[1,3,4]oxadiazol-2-yl)-cyclohexyl-phenyl-methanol

The title compound was obtained from (R)-cyclohexyl-mandelic acid as follows:

Step 1. 1,1′-Carbonyl diimidazole (6 g) was added to a stirred suspension of (R)-cyclohexyl-hydroxy-phenyl-acetic acid (8 g) in dry DCM (280 mL) at room temperature. After stirring for 2 h a solution of dimethylamino-acetic acid hydrazide (4 g) in dry DCM (40 mL) was added rapidly dropwise. After stirring at room temperature for three days, the reaction mixture was diluted with DCM and saturated sodium bicarbonate solution. The organic layer was dried (MgSO₄), filtered and evaporated in vacuo. Purification by silica gel chromatography eluting with 20-100% ethyl acetate in DCM gave (R)-cyclohexyl-hydroxy-phenyl-acetic acid N′-(2-dimethylaminoacetyl)-hydrazide (5.13 g, 45%) as a white foam. ¹H NMR (400 MHz, DMSO-d₆): δ 9.51 (2H, brs), 7.55-7.6 (2H, m), 7.28-7.36 (2H, m), 7.18-7.25 (1H, m), 5.53 (1H, s), 2.88 (2H, s), 2.2-2.8 (1H, m), 2.2 (6H, s), 1.68-1.79 (2H, m), 1.56-1.63 (2H, m), 1.0-1.38 (6H, m).

Step 2. A solution of the foregoing compound (5.13 g) in acetic anhydride (65 mL) was heated at 90° C. for 1 h. The reaction mixture was cooled and poured into an ice-water-NaHCO₃ mixture. More NaHCO₃ solution was added until the mixture was basic. The mixture was extracted with DCM and the organic phase was washed with brine, dried (Na₂SO₄), filtered and evaporated in vacuo to give a crude solid. Purification by silica gel chromatography (eluting with 20-100% ethyl acetate in DCM and 0-20% methanol in DCM) gave (R)-cyclohexyl-(5-dimethylaminomethyl-[1,3,4]oxadiazol-2-yl)-phenyl-methanol (3.29 g, 69%) as a yellow foam. LCMS (Method 7, 2.37 min). MH⁺=316.

Step 3. Reaction of the foregoing compound (1 g) with cyanogen bromide as described for Intermediate 14 gave the title compound (0.26 g, 23%) as a white foam. Data for the title compound: LCMS (Method 7, 3.90 min). MH⁺=353.

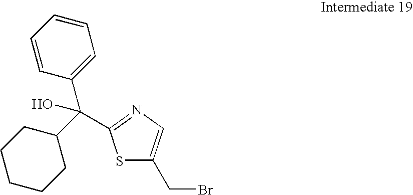

### (RS)-(5-Bromomethyl-thiazol-2-yl)-cyclohexyl-phenyl-methanol

Step 1. A solution of 5-methylthiazole (2.5 g, 25.2 mmol) in anhydrous THF (50 mL) was cooled to −78° C. under a N₂ atmosphere. n-Butyllithium (11 mL of a 2.5 M solution in hexanes) was added dropwise over 5 minutes. The reaction was stirred for 10 minutes before a solution of cyclohexylphenyl ketone in anhydrous THF (50 mL) was added dropwise. The reaction was stirred for 30 minutes at −78° C. then allowed to warm to ambient temperature before being quenched by addition of sat. Na₂CO₃ and extracted into diethyl ether. The combined organic extracts were washed with brine, dried (MgSO₄) and concentrated. Purification by chromatography using 0-10% EtOAc/heptanes gave cyclohexyl-(5-methyl-thiazol-2-yl)-phenyl-methanol (6.15 g, 85%). ¹H NMR (300 MHz, CDCl3): δ 7.6-7.7 (2H, m), 7.15-7.40 (4H, m), 3.8 (1H, s), 2.30-2.40 (4H, m), 1.6-1.8 (3H, m), 1.35-1.45 (2H, m), 1.05-1.3 (5H, m).

Step 2. A suspension of the foregoing compound (0.5 g), N-bromosuccinimide (0.35 g) and AIBN (50 mg) in CCl₄ (50 mL) was heated at 120° C. for 90 min. The reaction mixture was cooled, filtered and evaporated in vacuo to give the title compound (600 mg, 94%) as a sticky foam. ¹H NMR (300 MHz, CDCl₃) δ 7.72-7.64 (2H, m), 7.63 (1H, s), 7.37-7.31 (2H, m), 7.27-7.22 (1H, m), 4.63 (2H, m), 3.45 (1H, s), 2.52-2.41 (1H, m), 1.78-1.04 (10H, m).

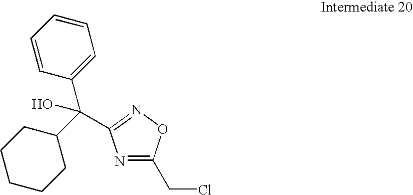

### (5-Chloromethyl-[1,2,4]oxadiazol-3-yl)-cyclohexyl-phenyl-methanol, Enantiomers 1 and 2

Step 1. Cyclohexylmagnesium chloride (14.08 mL, 2.0 M in ether) was added dropwise to a stirred solution of (5-chloromethyl-[1,2,4]-oxadiazol-3-yl)-phenyl-methanone (5.7 g) (H. Brachwitz, J. Prakt. Chem., 1969, 311, 661-70) in THF (120 mL) at −78° C. The reaction mixture was stirred for 1 h, then quenched with 1M HCl at −60° C. and allowed to warm to room temperature. The reaction mixture was extracted with ethyl acetate and the organic extract was washed with water and brine, dried (MgSO₄), filtered and evaporated in vacuo. Purification by silica gel chromatography (eluting with 10% ethyl acetate in isohexane) gave (5-chloromethyl-[1,2,4]oxadiazol-3-yl)-cyclohexyl-phenyl-methanol as a pale yellow oil (6.4 g, 82%) which solidified on standing. ¹H NMR (300 MHz, CDCl₃) 7.63-7.57 (2H, m), 7.38-7.31 (2H, m), 7.29-7.22 (1H, m), 4.64 (1H, s), 3.12 (2H, s), 2.38-2.25 (1H, m), 1.81-1.07 (10H, m).

Step 2. The foregoing compound (1 g) was separated into its enantiomers by chiral HPLC using an AD-H column (50×500 20 μM) eluting with 80% isohexane/20% 2-propanol; flow rate 236 mL/min; temperature 25° C., detection 220 nm. Enantiomer 1: 99.4% ee (380 mg). Enantiomer 2: 97.3% ee (393 mg).

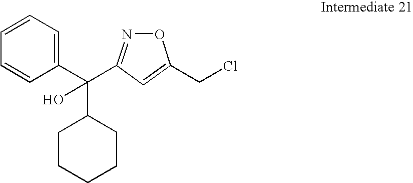

### (5-Chloromethyl-isoxazol-3-yl)-cyclohexyl-phenyl-methanol, Enantiomers 1 and 2

To a solution of (5-(chloromethyl)isoxazol-3-yl)(phenyl)methanone (7 g, 31.58 mmol) (Med. Chem. Res 10 (9), 615-633 (2001)) at −78° C. under nitrogen was added cyclohexyl magnesium chloride 2 M in diethyl ether (17.37 mL, 34.74 mmol) dropwise over 10 minutes. The reaction was stirred for 2 hours and then 1M HCl (aqueous, 200 mL) added. The mixture was allowed to warm to room temperature and then diluted with diethyl ether (200 mL). The organic layer was separated and the aqueous re-extracted with diethyl ether (2×100 mL). The combined organic extracts were dried over magnesium sulfate and concentrated under vacuum. Purification of the resultant crude oil was carried out by column chromatography on silica eluting with ethyl acetate/isohexane (10/90) to yield (5-(chloromethyl)isoxazol-3-yl)-cyclohexyl-phenyl-methanol as an off-white solid (5.60 g, 58.0%). The enantiomers were separated by preparative chiral HPLC of the racemate. Enantiomer 1: (Method 9; 20% ethanol/80% isohexane; Rt6.41 min): ¹H NMR (400 MHz, DMSO-d₆): δ 7.45-7.49 (2H, m), 7.31 (2H, t, J=7.7 Hz), 7.20 (1H, tt, J=7.3, 1.4 Hz), 6.50 (1H, s), 5.80 (1H, s), 4.89 (2H, s), 2.15-2.26 (1H, m), 1.51-1.78 (4H, m), 0.94-1.44 (6H, m). Enantiomer 2: (Method 9; 20% ethanol/80% isohexane; Rₜ, 10.1 min): ¹H NMR (400 MHz, DMSO-d₆): δ 7.43-7.52 (2H, m), 7.31 (2H, t, J=8.0 Hz), 7.20 (1H, dt, J=14.5, 1.3 Hz), 6.50 (1H, s), 5.80 (1H, s), 4.89 (2H, s), 2.14-2.25 (1H, m), 1.53-1.72 (4H, m), 0.96-1.33 (6H, m).

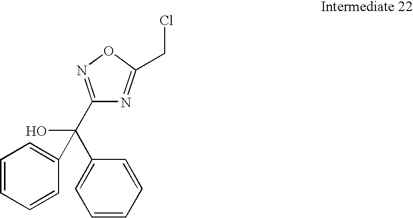

### (5-Chloromethyl-[1,2,4]oxadiazol-3-yl)-diphenyl-methanol

The title compound was obtained from amino-[N-hydroxyimino]-acetic acid ethyl ester as follows:

Step 1. Diisopropylamine (21.6 mL) was added dropwise to a stirred suspension of amino-[(Z)-hydroxyimino]-acetic acid ethyl ester (15 g) in dry DCM (300 mL) cooled to −10° C. After stirring for 10 minutes a solution of chloroacetyl chloride (9.96 mL) in dry DCM (30 mL) was added dropwise over 20 minutes to the cooled mixture. After stirring at room temperature overnight, the reaction was poured into ice/water mixture (1 L) to obtain two layers. Filtration of the bottom layer gave a brown precipitate which was washed with EtOAc (50 mL) and dried in vacuo to give amino-{N-[chloroacetate]imino}-acetic acid ethyl ester (21.54 g, 91%) as a cream solid. LCMS (Method 7): Rt 2.41 min, m/z 209 [MH]⁺.

Step 2. A thick suspension of amino-{N—[chloroacetate]imino}-acetic acid ethyl ester (11.43 g) in toluene (200 mL) was allowed to reflux in a Dean-Stark apparatus for 12 h. The reaction mixture was allowed to cool down to room temperature and dried with MgSO₄. Filtration of the solid residue and evaporation of the solvent in vacuo gave 5-chloromethyl-[1,2,4]oxadiazole-3-carboxylic acid ethyl ester as an oil. LCMS (Method 7): Rt 2.97 min, m/z 190 [no MH]+. ¹H NMR (400 MHz, DMSO-d₆): δ 5.17 (s, 2H), 4.45 (q, 2H), 1.28 (t, 3H).

Step 3. Phenylmagnesium bromide (3 M solution in diethyl ether; 13.44 mL) was added rapidly drop wise to a solution of 5-chloromethyl-[1,2,4]oxadiazole-3-carboxylic aid ethyl ester (3.48 g) in anhydrous THF (85 mL) at −78° C. under N₂. The reaction mixture was stirred at −78° C. for 45 mins, then was allowed to warm up to −20° C. then recooled to −78° C. and stirred for a further 30 mins. The reaction mixture was quenched with a chilled solution of 1 M HCl (40 mL), allowed to warm to room temperature and extracted with EtOAc (2×70 mL). The combined organic extracts were washed with water, NaHCO₃ and brine, dried (MgSO₄), filtered and evaporated in vacuo. Purification by silica gel chromatography eluting with 0-15% EtOAc/cyclohexane gave the title compound (2.96 g, 54%) as a cream solid. LCMS (Method 7): Rₜ 3.64 min (no [MH]⁺). ¹H NMR (400 MHz, DMSO-d₆): δ 7.34-7.24 (m, 10H), 7.09-7.05 (m, 1H), 5.10 (s, 2H).

The following Intermediates were made from 3-R-quinuclidinol by Procedure B:

| | | Method |
|---|---|---|
| Intermediate | | Rt |
| Number | Structure | MH⁺ |
| 23 | 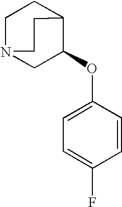 | 7 2.05 min 222 |
| 24 | 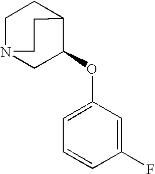 | 7 2.09 min 222 |
| 25 | 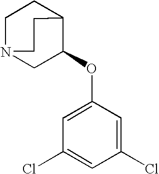 | 7 2.38 min 274 |
| 26 | 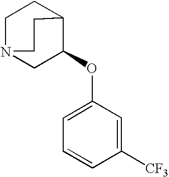 | 7 2.28 min 272 |
| 27 | 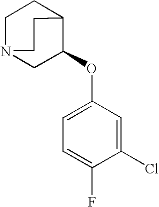 | 7 2.26 min 256 |
| 28 | 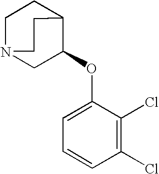 | 7 2.37 min 274 |
| 29 | 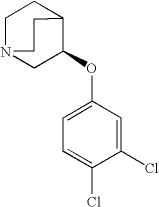 | 7 2.36 min 274 |
| 30 | 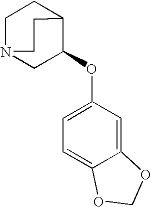 | 7 2.05 min 248 |
| 31 | 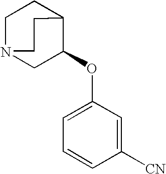 | 7 2.05 min 229 |
| 32 | 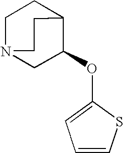 | 7 1.94 min 210 |
| 33 | 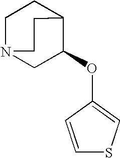 | 7 1.95 min 210 |

### General Procedure A

### Preparation of 3-R-benzyloxyquinuclidine

Step 1. A solution of borane-THF (1.0 M in THF, 24.8 mL) was added dropwise to a solution of R-3-quinuclidinol (3 g) in THF (20 mL) at 0° C. The reaction mixture was allowed to warm to room temperature, stirred for 24 h, and evaporated in vacuo. The resulting residue was diluted with chloroform, washed with water, brine, dried (MgSO₄), filtered and evaporated in vacuo. The resulting residue was dissolved in ether and treated with 0-50% DCM/petroleum spirit (bp 40-60° C.). The resulting fine precipitate was collected by filtration to afford (R)-1-boranyl-1-aza-bicyclo[2.2.2]octan-3-ol (2.1 g, 63%). ¹H NMR (400 MHz, CDCl₃): δ 4.02-3.97 (1H, m), 3.19-3.12 (1H, m), 2.98-2.67 (6H, m), 2.11-2.01 (1H, m), 1.83-1.75 (1H, m), 1.66-1.27 (3H, m).

Step 2. A solution of the foregoing compound (360 mg) in DMF (5 mL) was treated with NaH (101 mg of 60% dispersion in mineral oil), stirred for 5 mins and then treated with benzyl bromide (0.302 mL). The reaction mixture was stirred overnight at room temperature, evaporated in vacuo and purified by silica gel chromatography (eluting with petroleum spirit 40-60° C./DCM [1:0 to 1:1]) to give (R)-3-benzyloxy-1-boranyl-1-aza-bicyclo[2.2.2]octane as a clear oil (433 mg, 74%). ¹H NMR (400 MHz, CDCl₃): δ 7.37-7.24 (5H, m), 4.52 (1H, d, J=11.6), 4.44 (1H, d, J=11.6), 3.74-3.67 (1H, m), 3.23-2.78 (7H, m), 2.26-2.20 (1H, m), 2.13-2.01 (1H, m), 1.87-1.76 (1H, m), 1.64-1.49 (1H, m).

Step 3. A solution of the foregoing compound (433 mg) in acetone (5 mL) at 0° C. was treated with 1.25 M HCl-MeOH (10.45 mL), stirred at 0° C. for 0.5 h, and then stirred at room temperature for 0.5 h. The reaction mixture was evaporated in vacuo and purified by silica gel chromatography (eluting with 0-50% DCM/methanol) to afford the title compound (399 mg, 69%) as a white solid. LCMS (Method 7, Rt 0.35 min). MH⁺=218.

### General Procedure B

### Preparation of 3-(3-fluorophenoxy)-quinuclidine

A solution of R-3-quinuclidinol (1.25 g), CuI (93.1 mg), 1,10-phenanthroline (176 mg), Cs₂CO₃ (3.19 g) and 3-fluoro-iodo-benzene (1.11 g) in toluene (2.5 mL) was heated at 100° C. for 20 h. The reaction mixture was cooled, diluted with ethyl acetate and filtered through Celite. The insoluble material was washed several times with ethyl acetate. The filtrate was washed with water, dried (MgSO₄), filtered and evaporated in vacuo. Purification by SCX and silica gel chromatography (DCM-[2M NH₃-methanol] mixtures) gave (R)-3-(3-fluoro-phenoxy)-1-aza-bicyclo[2.2.2]octane (490 mg, 45%) as a brown oil. LCMS (Method 7, Rt 2.09 min). MH⁺=222.

### General Procedure C1

### Preparation of (RS)-(3-benzylsulfanyl-1-aza-bicyclo[2.2.2]octane from 3-chloroquinuclidine hydrochloride

Benzyl mercaptan (3.87 mL) was dissolved in DMF (20 mL) and treated cautiously with NaH (2.64 g of 60% dispersion in mineral oil). After 20 mins, the reaction mixture was treated with 3-chloroquinuclidine hydrochloride (5 g) and the reaction mixture was heated at 100° C. for 18 h. The reaction mixture was diluted with water and extracted with ethyl acetate. The organic layer was washed with brine, dried (MgSO₄), filtered and evaporated in vacuo. Purification by SCX and silica gel chromatography (eluting with 2-5% [2M NH₃ in methanol]: DCM) gave (RS)-(3-benzylsulfanyl-1-aza-bicyclo[2.2.2]octane (3.3 g, 52%) as a colourless oil. ¹H NMR (300 MHz, CDCl₃): δ7.33-7.20 (5H, m), 3.69 (2H, s), 3.18 (1H, ddd, J=13.86, 9.60, 2.37 Hz), 2.96-2.66 (5H, m), 2.55 (1H, ddd, J=13.87, 6.00, 2.20 Hz), 2.09-1.95 (1H, m), 1.85-1.78 (2H, m), 1.52-1.30 (2H, m).

### General Procedure C2

### Preparation of (R)-(3-benzylsulfanyl-1-aza-bicyclo[2.2.2]octane from methanesulfonic acid (S)-(1-aza-bicyclo[2.2.2]oct-3-yl) ester

Benzyl mercaptan (0.34 mL) was added slowly to a suspension of NaH (234 mg of 60% dispersion in mineral oil) in DMF (1 mL) at 0° C. After 10 mins, the reaction mixture was treated with methanesulfonic acid (S)-(1-aza-bicyclo[2.2.2]oct-3-yl) ester (0.5 g) (J. Med. Chem., 1992, 35, 2392-2406) and the reaction mixture was heated at 100° C. for 20 min. The reaction mixture was diluted with NaHCO₃ solution and extracted with ethyl acetate. The organic layer was washed with brine, dried (MgSO₄), filtered and evaporated in vacuo. Purification by SCX gave (R)-3-benzylsulfanyl-1-aza-bicyclo[2.2.2]octane (37 mg, 6.5%) as a colourless oil.

### General Procedure D

### Preparation of 3-benzamidoquinuclidine

A solution of 3-R-amino-quinuclidine dihydrochloride (550 mg) in THF (10 mL) and DMF (5 mL) was treated with DIPEA (1.41 mL) and benzoyl chloride (0.321 mL) and stirred at room temperature overnight. The reaction mixture was evaporated in vacuo and purified by silica gel chromatography (eluting with 10% methanol/DCM) to give (R)—N-(1-aza-bicyclo[2.2.2]oct-3-yl)-benzamide (375 mg, 87%) as a white solid. LCMS (Method 7, Rt 0.32 min). MH⁺=231

### General Procedure E

### Preparation of (3-phenethyl)quinuclidine

Step 1. A solution of 3-quinuclidinone hydrochloride (16 g) was treated with saturated aqueous NaHCO₃ solution. The reaction mixture was extracted twice with ether and twice with DCM. The combined organic extracts were dried (MgSO₄), filtered and evaporated in vacuo to give the free base as a white solid. This was dissolved in ether (20 mL) and added slowly at 0° C. to a solution of phenethylmagnesium bromide (1.0 M in THF, 100 mL) in ether (100 mL). The reaction mixture was stirred at room temperature for 4 h, cooled to 0° C. and quenched carefully by the addition of water. The reaction mixture was allowed to warm to room temperature, evaporated in vacuo and extracted with CHCl₃/2-propanol (10:1). The organic extracts were evaporated in vacuo to give a to crude residue. Purification by SCX gave 3-phenethyl-1-aza-bicyclo[2.2.2]octan-3-ol (5.5 g, 24%) as a white solid.

Step 2. A solution of the foregoing compound (1 g) was treated with SOCl₂ (5 mL) causing it to dissolve and gas to be evolved. The reaction mixture was evaporated in vacuo and triturated with ether to give a mixture of 3-[2-phenyl-eth-(E)-ylidene]-1-aza-bicyclo[2.2.2]octane, 3-[2-phenyl-eth—(Z)-ylidene]-1-aza-bicyclo[2.2.2]octane and 3-phenethyl-1-aza-bicyclo[2.2.2]oct-2-ene (1 g, 93%) as a white solid.

Step 3. A solution of the foregoing mixture (1 g) and Pd—C (10%, 0.3 g) in ethanol (15 mL) was stirred under an atmosphere of hydrogen at room temperature for 5 h. The reaction mixture was filtered and purified by SCX and column chromatography (eluting with 2-6% [2 M NH₃ in methanol]-DCM) to give the title compound (0.44 g, 51%) as a colourless oil. ¹H NMR (300 MHz, CDCl₃): δ7.32-7.25 (2H, m), 7.22-7.16 (3H, m), 3.14-3.04 (1H, m), 2.88-2.76 (4H, m), 2.59 (2H, appt, J=7.83 Hz), 2.39 (1H, ddd, J=13.42, 6.00, 2.25 Hz), 1.76-1.33 (8H, m).

### General Procedure F

### Formation of Quaternary Ammonium Salts from Quinuclidines and Alkyl Halides

An equimolar solution of a quinuclidine (0.1 mmol) and alkylating agent (0.1 mmol) was heated at 50° C. in MeCN (0.8 mL) overnight. If the resulting product precipitated, it was collected by filtration, washed with ethyl acetate and ether and dried in vacuo; or if it did not precipitate, the reaction mixture was evaporated and the product isolated by silica gel chromatography and/or HPLC.

### Example 1

### 1-[2-(Hydroxy-diphenyl-methyl)-oxazol-5-ylmethyl]-3-phenoxy-1-azoniabicyclo[2.2.2]octane bromide, enantiomer 1

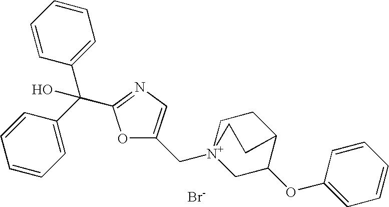

To a solution of (5-bromomethyl-oxazole-2-yl)-diphenyl-methanol (0.119 g, 0.344 mmol) in acetonitrile (1 mL) and chloroform (1 mL) was added 3-phenoxy quinuclidine enantiomer 1 (0.014 g, 0.069 mmol). After heating at 50° C. for 48 h the reaction mixture was cooled to room temperature and the solvents evaporated. The residue was purified by column chromatography using 0-15% MeOH/DCM to give the title compound (13 mg, 34%). LCMS (Method 1): Rₜ 7.72 min, m/z 467 [M-Br]+. ¹H NMR, 400 MHz, CD₃OD: δ 7.50 (1H, s), 7.25-7.38 (12H, m), 6.99-7.04 (1H, m), 6.90-6.95 (2H, m), 4.85-4.93 (1H, m), 4.63-4.72 (2H, m), 3.85-3.95 (1H, m), 3.35-3.58 (5H, m), 2.50-2.55 (1H, m), 2.28-2.40 (1H, m), 2.08-2.18 (1H, m), 1.88-2.05 (2H, m).

### Example 2

### 1-[2-(Hydroxy-diphenyl-methyl)-oxazol-5-ylmethyl]-3-phenoxy-1-azoniabicyclo[2.2.2]octane bromide, enantiomer 2

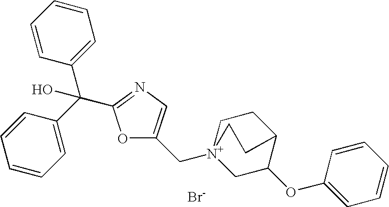

A solution of (5-bromomethyl-oxazole-2-yl)-diphenyl-methanol (0.102 g, 0.295 mmol) and 3-phenoxy quinuclidine enantiomer 2 (0.012 g, 0.059 mmol) was reacted in a similar manner to that described in example 1 to give the title compound (11 mg, 34%). LCMS (Method 1): Rₜ 7.68 min, m/z 467 [M-Br]+. ¹H NMR, (400 MHz, CD₃OD): δ 7.50 (1H, s), 7.25-7.38 (12H, m), 6.99-7.04 (1H, m), 6.90-6.95 (2H, m), 4.85-4.93 (1H, m), 4.61-4.72 (2H, m), 3.83-3.92 (1H, m), 3.38-3.58 (5H, m), 2.50-2.56 (1H, m), 2.28-2.39 (1H, m), 2.08-2.18 (1H, m), 1.88-2.05 (2H, m).

### Example 3

### 1-[2-[(RS)—(Cyclohexyl-hydroxy-phenyl-methyl)]-oxazol-5-ylmethyl]-3-phenoxy-1-azonia-bicyclo[2.2.2]octane bromide, enantiomer 1

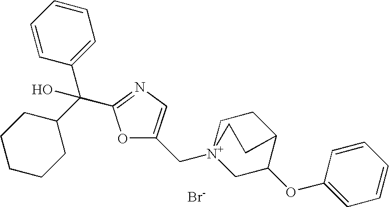

A solution of (5-bromomethyl-oxazole-2-yl)-cyclohexyl-phenyl-methanol racemate (0.021 g, 0.060 mmol) and 3-phenoxy quinuclidine enantiomer 1 (0.010 g, 0.050 mmol) was reacted in a similar manner to that described in example 1 to give the title compound (9 mg, 32%). LCMS (Method 1): Rₜ 8.62 min, m/z 473 [M-Br]+. ¹H NMR, (400 MHz, CD₃OD): δ 7.50-7.55 (2H, m), 7.45 (1H, s), 7.21-7.35 (5H, m), 6.99-7.05 (1H, m), 6.90-6.96 (2H, m), 4.80-4.95 (1H, m), 4.60-4.68 (2H, m), 3.83-3.93 (1H, m), 3.38-3.57 (5H, m), 2.50-2.55 (1H, m), 2.26-2.45 (2H, m), 2.07-2.18 (1H, m), 1.87-2.05 (2H, m), 1.60-1.80 (3H, m), 1.50-1.59 (1H, m), 1.05-1.40 (6H, m).

### Example 4

### 1-[2-[(RS)—(Cyclohexyl-hydroxy-phenyl-methyl)]-oxazol-5-ylmethyl]-3-phenoxy-1-azonia-bicyclo[2.2.2]octane bromide, enantiomer 2

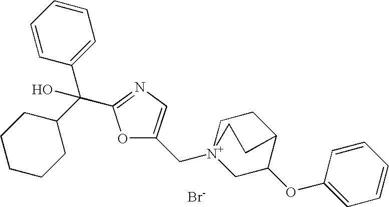

A solution of (5-bromomethyl-oxazole-2-yl)-cyclohexyl-phenyl-methanol racemate (0.021 g, 0.060 mmol) and 3-phenoxy quinuclidine enantiomer 2 (0.010 g, 0.050 mmol) was reacted in a similar manner to that described in example 1 to give the title compound (11 mg, 33%). LCMS (Method 1): Rₜ 8.57 min, m/z 473 [M-Br]+. ¹H NMR, (400 MHz, CD₃OD): δ 7.50-7.55 (2H, m), 7.45 (1H, s), 7.21-7.36 (5H, m), 6.99-7.04 (1H, m), 6.90-6.96 (2H, m), 4.80-4.95 (1H, m), 4.59-4.68 (2H, m), 3.83-3.93 (1H, m), 3.38-3.57 (5H, m), 2.50-2.55 (1H, m), 2.26-2.43 (2H, m), 2.07-2.18 (1H, m), 1.87-2.05 (2H, m), 1.60-1.80 (3H, m), 1.50-1.59 (1H, m), 1.05-1.40 (6H, m).

### Example 5

### 1-[2-((S))-Cyclohexyl-hydroxy-phenyl-methyl)-oxazol-5-ylmethyl]-1-azonia-bicyclo[2.2.2]octane bromide

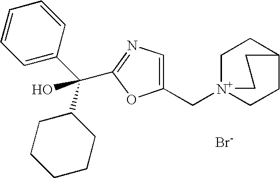

A microwave reaction vessel was charged with [2-((S)-cyclohexyl-hydroxy-phenyl-methyl)-oxazol-5-ylmethyl]-trimethyl-ammonium bromide (20 mg, 0.049 mmol), quinuclidine (55 mg, 0.490 mmol), acetonitrile (0.9 mL) and chloroform (0.6 mL). The vessel was sealed and irradiated under microwave heating at 180° C. for 30 min. The reaction was allowed to cool to room temperature. LCMS (Method 2): Rt 2.29 min, m/z 381 [M⁺].

### Example 6

### (S)-1-[2-((S)—Cyclohexyl-hydroxy-phenyl-methyl)-oxazol-5-ylmethyl]-3-phenoxy-1-azonia-bicyclo[2.2.2]octane bromide

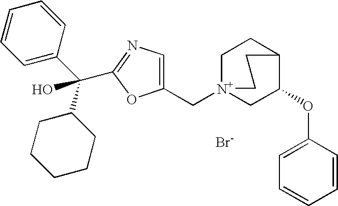

### Method A:

A microwave reaction vessel was charged with [2-((S)-cyclohexyl-hydroxy-phenyl-methyl)-oxazol-5-ylmethyl]-trimethyl-ammonium bromide (20 mg, 0.049 mmol), (S)-3-phenoxy-1-aza-bicyclo[2.2.2]octane (99 mg, 0.490 mmol), acetonitrile (0.9 mL) and chloroform (0.6 mL). The vessel was sealed and irradiated under microwave heating at 180° C. for 30 min. The reaction was allowed to cool to room temperature. LCMS (Method 2): Rt 2.64 min, m/z 473 [M⁺]. ¹H NMR (400 MHz, CDCl₃): δ 8.73 (s, 1H), 7.54 (d, 2H), 7.36 (s, 1H), 7.29 (d, 2H), 7.25-7.14 (m, 3H), 6.99 (t, 1H), 6.82 (d, 2H), 4.80 (s, 2H), 4.40-4.31 (m, 1H), 3.83 (s, 2H), 3.60 (t, 2H), 3.48 (d, 2H), 3.16 (d, 2H), 2.38 (s, 1H), 2.27 (s, 2H), 2.25-2.13 (m, 1H), 1.91 (d, 3H), 1.88-1.66 (m, 3H), 1.26 (d, 3H), 1.13-1.04 (m, 2H).

### Method B:

A reaction vessel was charged with [2-((S)-cyclohexyl-hydroxy-phenyl-methyl)-oxazol-5-ylmethyl]-trimethyl-ammonium bromide (20 mg, 0.049 mmol), (S)-3-phenoxy-1-aza-bicyclo[2.2.2]octane (99 mg, 0.490 mmol), acetonitrile (0.9 mL) and chloroform (0.6 mL) and heated at 50° C. for 48 h. The reaction contents were allowed to cool to room temperature. LCMS (Method 2): Rt 2.64 min, m/z 473 [M⁺].

### Example 7

### (R)-1-[2-((S)—Cyclohexyl-hydroxy-phenyl-methyl)-oxazol-5-ylmethyl]-3-phenoxy-1-azonia-bicyclo[2.2.2]octane bromide

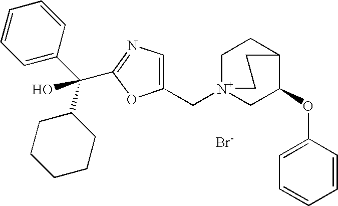

The title compound (40.2 mg, 51%) was made from R-phenoxyquinuclidinol and (S)-(5-bromomethyl-oxazol-2-yl)-cyclohexyl-phenyl-methanol [prepared from Intermediate 11 by the procedure described for the synthesis of Intermediate 14] according to General Procedure F. Data for the title compound: MS (Method 6): Rt 8.17 min, m/z 473 [M⁺]. ¹H NMR (400 MHz, CD₃OD): δ 7.53-7.49 (m, 2H), 7.46 (s, 1H), 7.34-7.28 (m, 4H), 7.27-7.21 (m, 1H), 7.03-6.98 (m, 1H), 6.95-6.91 (m, 2H), 4.90 (d, 1H), 4.69-4.58 (m, 2H), 3.90 (ddd, 1H), 3.57-3.41 (m, 5H), 2.54-2.50 (m, 1H), 2.42-2.27 (m, 2H), 2.18-2.07 (m, 1H), 2.07-1.87 (m, 2H), 1.79-1.59 (m, 3H), 1.53 (d, 1H), 1.40-1.02 (m, 7H).

### Example 8

### (S)-1-[2-((R)—Cyclohexyl-hydroxy-phenyl-methyl)-oxazol-5-ylmethyl]-3-phenoxy-1-azonia-bicyclo[2.2.2]octane; bromide

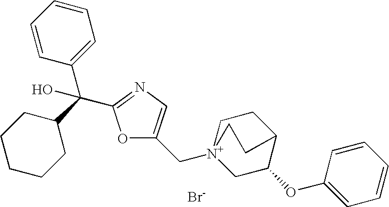

The title compound (28 mg, 36%) was prepared according to General Procedure F. Data for the title compound: LCMS (Method 6, 8.10 min). M⁺=473. ¹H NMR (400 MHz, CD₃OD): δ 7.53-7.49 (m, 2H), 7.46 (s, 1H), 7.34-7.28 (m, 4H), 7.27-7.21 (m, 1H), 7.03-6.98 (m, 1H), 6.95-6.91 (m, 2H), 4.89 (s, 1H), 4.69-4.57 (m, 2H), 3.88 (ddd, 1H), 3.56-3.46 (m, 2H), 3.47-3.38 (m, 4H), 2.55-2.51 (m, 1H), 2.43-2.26 (m, 2H), 2.18-2.07 (m, 1H), 2.07-1.87 (m, 2H), 1.80-1.60 (m, 3H), 1.54 (d, 1H), 1.40-1.05 (m, 6H).

### Example 9

### (R)-[2-((R)—Cyclohexyl-hydroxy-phenyl-methyl)-oxazol-5-ylmethyl]-3-phenoxy-1-azonia-bicyclo[2.2.2]octane; bromide

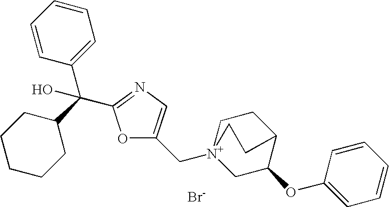

The title compound (62 mg, 75%) was prepared according to General Procedure F. Data for the title compound: LCMS (Method 6, Rt 8.02 min). M⁺=473. ¹H NMR (400 MHz, CD₃OD): δ 7.53-7.49 (m, 2H), 7.46 (s, 1H), 7.34-7.28 (m, 4H), 7.27-7.21 (m, 1H), 7.03-6.98 (m, 1H), 6.95-6.91 (m, 2H), 4.89 (s, 1H), 4.69-4.57 (m, 2H), 3.88 (ddd, 1H), 3.56-3.46 (m, 2H), 3.47-3.38 (m, 4H), 2.55-2.51 (m, 1H), 2.43-2.26 (m, 2H), 2.18-2.07 (m, 1H), 2.07-1.87 (m, 2H), 1.80-1.60 (m, 3H), 1.54 (d, 1H), 1.40-1.05 (m, 6H).

### Example 10

### (S)-3-benzoyloxy-1-[2-((R)-cyclohexyl-hydroxy-phenyl-methyl)-oxazol-5-ylmethyl]-1-azonia-bicyclo[2.2.2]octane; formate

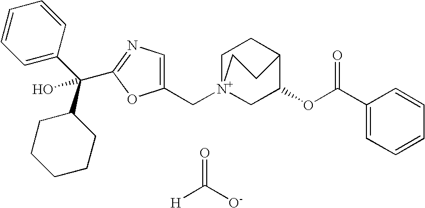

The title compound was prepared from benzoic acid (S)-(1-aza-bicyclo[2.2.2]oct-3-yl) ester (Eur. J. Org. Chem., 2003, 295) and Intermediate 14 by application of General Procedure F. Data for the title compound: LCMS (Method 1, Rt 8.56 min). M⁺=501. ¹H NMR (400 MHz, CDCl₃): δ 8.71 (s, 1H), 7.98-7.93 (m, 2H), 7.63-7.53 (m, 3H), 7.50-7.40 (m, 3H), 7.23 (d, 2H), 7.13 (t, 1H), 5.30 (s, 1H), 5.07-4.90 (m, 2H), 4.15 (dd, 1H), 3.90-3.77 (m, 1H), 3.70 (s, 2H), 3.44 (d, 1H), 3.37 (d, 1H), 2.46 (s, 1H), 2.29 (s, 1H), 2.16 (s, 1H), 2.03 (s, 2H), 1.95 (s, 3H), 1.70-1.53 (m, 3H), 1.35-1.22 (m, 3H), 1.12 (d, 3H).

### Example 11

### 1-[2-((R)-cyclohexyl-hydroxy-phenyl-methyl)-oxazol-5-ylmethyl]-4-phenyl-1-azonia-bicyclo[2.2.2]octane; formate

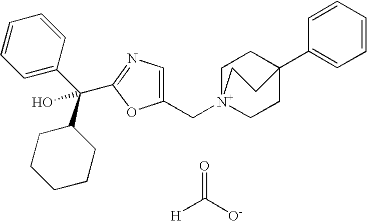

The title compound was prepared from 4-phenyl-1-aza-bicyclo[2.2.2]octane and Intermediate 14 by application of General Procedure F. Data for the title compound: LCMS (Method 1, Rt 8.34 min). M⁺=457. ¹H NMR (400 MHz, CDCl₃): δ 8.75 (s, 1H), 7.58 (d, 2H), 7.44 (s, 1H), 7.34 (t, 2H), 7.32-7.24 (m, 3), 7.23-7.15 (m, 3H), 4.99 (d, 1H), 4.88 (d, 1H), 3.66 (s, 8H), 2.13 (t, 6H), 1.76-1.61 (m, 3H), 1.29 (d, 4H), 1.17-1.06 (m, 3H).

### Example 12

### (R)-3-(Benzyloxy)-1-[2-(R)-cyclohexyl-hydroxy-phenyl-methyl)-oxazol-5-ylmethyl]-1-azonia-bicyclo[2.2.2]octane; bromide

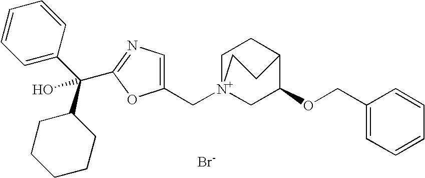

Step 1. (R)-3-(benzyloxy)-1-aza-bicyclo[2.2.2]octane was prepared from benzyl bromide and 3-R-quinuclidinol by the procedure described in Procedure A.

Step 2. The title compound was prepared from the foregoing compound and Intermediate 14 by application of General Procedure F. Data for the title compound: LCMS (Method 1, Rt 8.66 min). M⁺=487. ¹H NMR (400 MHz, CDCl₃): δ 7.58-7.53 (m, 2H), 7.48 (s, 1H), 7.39-7.27 (m, 3H), 7.25 (d, 4H), 7.16 (t, 1H), 5.16-4.95 (m, 2H), 4.50-4.40 (m, 2H), 4.34 (s, 1H), 4.28-4.20 (m, 1H), 4.09-3.89 (m, 2H), 3.84-3.73 (m, 2H), 3.21-3.10 (m, 2H), 2.32 (d, 2H), 2.17-2.08 (m, 1H), 2.03-1.94 (m, 1H), 1.86 (d, 3H), 1.69 (d, 2H), 1.36-1.17 (m, 4H), 1.20-1.03 (m, 3H).

### Example 13

### (R)-3-benzoylamino-1-[2-((R)-cyclohexyl-hydroxy-phenyl-methyl)-oxazol-5-ylmethyl]-1-azonia-bicyclo[2.2.2]octane; formate

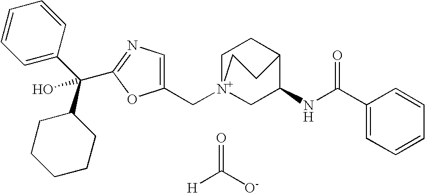

Step 1. (R)—N-(1-aza-bicyclo[2.2.2]oct-3-yl)-benzamide was prepared from benzoyl chloride and 3-R-aminoquinuclidine dihydrochloride by the procedure described in Procedure D.

Step 2. The title compound was prepared from the foregoing compound and Intermediate 14 by application of General Procedure F. Data for the title compound: LCMS (Method 1, Rt 7.77 min). M⁺=500. ¹H NMR (400 MHz, CDCl₃): δ 9.60 (d, 1H), 8.57 (s, 1H), 7.99-7.94 (m, 2H), 7.48 (d, 2H), 7.43-7.36 (m, 1H), 7.32 (t, 2H), 7.28 (s, 1H), 7.27-7.19 (m, 2H), 7.16 (t, 1H), 4.63-4.35 (m, 3H), 4.30 (d, 1H), 4.11 (s, 1H), 3.42 (t, 1H), 3.15 (s, 4H), 2.33 (d, 2H), 2.32-2.20 (m, 1H), 1.98-1.84 (m, 2H), 1.68 (s, 2H), 1.55 (s, 2H), 1.23 (s, 4H), 1.16-0.98 (m, 3H).

### Example 14

### (R)-3-benzoyloxy-1-[2-((R)-cyclohexyl-hydroxy-phenyl-methyl)-oxazol-5-ylmethyl]-1-azonia-bicyclo[2.2.2]octane; formate

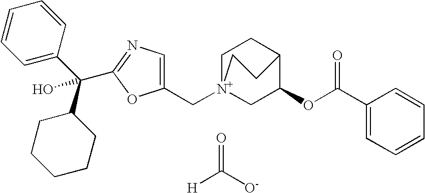

The title compound was prepared from benzoic acid (R)-(1-aza-bicyclo[2.2.2]oct-3-yl) ester (Eur. J. Org. Chem., 2003, 295) and Intermediate 14 by application of General Procedure F. Data for the title compound: LCMS (Method 1, Rt 8.39 min). M⁺=501. ¹H NMR (400 MHz, CDCl₃): δ 8.63 (s, 1H), 7.95 (d, 2H), 7.63-7.53 (m, 3H), 7.48-7.41 (m, 3H), 7.27 (d, 1H), 7.22 (s, 1H), 7.13 (t, 1H), 5.28 (s, 1H), 4.91 (s, 2H), 4.36 (s, 5H), 4.16-4.06 (m, 1H), 3.74 (s, 1H), 3.62 (s, 2H), 3.41 (d, 1H), 3.33 (d, 1H), 2.43 (s, 1H), 2.28 (s, 1H), 2.13 (s, 1H), 2.00 (s, 1H), 1.92 (d, 3H), 1.31-1.20 (m, 2H), 1.07 (d, 3H).

### Example 15

### (S)-3-Benzoylamino-1-[2-((R)-cyclohexyl-hydroxy-phenyl-methyl)-oxazol-5-ylmethyl]-1-azonia-bicyclo[2.2.2]octane; formate

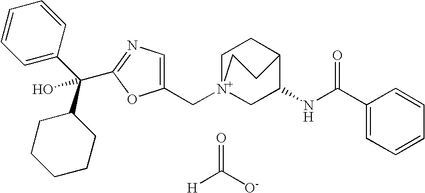

Step 1. (S)—N-(1-aza-bicyclo[2.2.2]oct-3-yl)-benzamide was prepared from benzoyl chloride and 3-S-aminoquinuclidine dihydrochloride by the procedure described in Procedure D.

Step 2. The title compound was prepared from the foregoing compound and Intermediate 14 by application of General Procedure F. Data for the title compound: LCMS (Method 1, Rt 7.75 min). M⁺=500. ¹H NMR (400 MHz, CDCl₃): δ 9.77 (d, 1H), 8.66 (s, 1H), 8.06 (d, 2H), 7.54-7.36 (m, 5H), 7.30 (d, 3H), 7.25-7.18 (m, 1H), 4.56 (d, 4H), 4.41 (s, 1H), 3.47-3.36 (m, 2H), 3.20 (t, 2H), 3.13 (s, 1H), 2.49-2.24 (m, 4H), 2.08-1.92 (m, 1H), 1.81-1.56 (m, 4H), 1.58 (d, 1H), 1.35-1.23 (m, 3H), 1.13 (d, 3 H).

### Example 16

### 3-benzyloxy-1-[2-((R)-cyclohexyl-hydroxy-phenyl-methyl)-oxazol-5-ylmethyl]-1-azonia-bicyclo[2.2.2]octane; formate

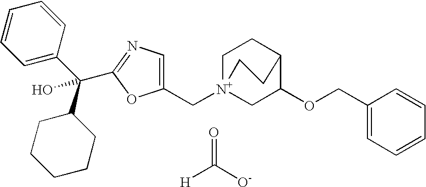

Step 1. (RS)-3-(benzyloxy)-1-aza-bicyclo[2.2.2]octane was prepared from benzyl bromide and 3-RS-quinuclidinol by the procedure described in Procedure A. Enantiomerically pure (S)-3-(benzyloxy)-1-aza-bicyclo[2.2.2]octane was obtained by separation of the foregoing mixture of enantiomers by means of chiral HPLC Method 5 (eluent: 99.9% ethanol, 0.1% DEA). Retention time for (S)-3-(benzyloxy)-1-aza-bicyclo[2.2.2]octane (the first eluting enantiomer) is 12.18 min.

Step 2. The title compound was prepared from the foregoing compound and Intermediate 14 by application of General Procedure F. Data for the title compound: LCMS (Method 6, Rt 8.47 min). M⁺=487. ¹H NMR (400 MHz, CDCl₃): δ 8.63 (s, 1H), 7.55-7.50 (m, 2H), 7.34-7.25 (m, 4H), 7.25-7.16 (m, 4H), 7.13 (t, 1H), 4.85-4.63 (m, 2H), 4.43-4.33 (m, 2H), 3.85 (s, 2H), 3.43 (s, 3H), 3.12 (q, 1H), 3.00 (d, 1H), 2.24 (s, 2H), 2.16-2.04 (m, 1H), 1.86 (s, 2H), 1.67 (d, 1H), 1.47 (d, 1H), 1.31-1.03 (m, 6H).

### Example 17

### (S)-1-[2-((R)—Cyclohexyl-hydroxy-phenyl-methyl)-oxazol-5-ylmethyl]-3-(3-trifluoromethyl-benzoylamino)-1-azonia-bicyclo[2.2.2]octane; bromide

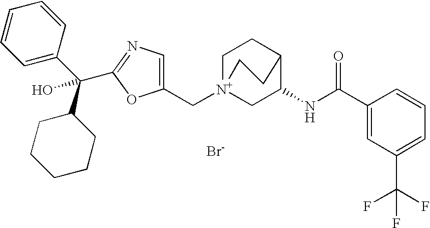

Step 1. N—(S)-1-aza-bicyclo[2.2.2]oct-3-yl-3-trifluoromethyl-benzamide was prepared from 3-trifluoromethyl-benzoyl chloride and 3-S-aminoquinuclidine dihydrochloride by the procedure described in Procedure D. Data for N—(S)-1-aza-bicyclo[2.2.2]oct-3-yl-3-trifluoromethyl-benzamide: LCMS (Method 7, Rt 1.94 min). MH⁺=299.

Step 2. The title compound was prepared from the foregoing compound and Intermediate 14 by application of General Procedure F. Data for the title compound: LCMS (Method 6, Rt 8.27 min). M⁺=568. ¹H NMR (400 MHz, CD₃OD); δ 8.17-8.08 (m, 2H), 7.85 (d, 1H), 7.67 (t, 1H), 7.51 (d, 3H), 7.46 (s, 1H), 7.28 (t, 2H), 7.18 (t, 1H), 4.60 (s, 2H), 4.42 (s, 1H), 3.95 (ddd, 1H), 3.53-3.28 (m, 5H), 2.40-2.33 (m, 2H), 2.28 (d, 1H), 2.10 (dd, 2H), 1.96 (t, 1H), 1.73-1.46 (m, 5H), 1.35-0.98 (m, 6H).

### Example 18

### (S)-1-[2-((R)-cyclohexyl-hydroxy-phenyl-methyl)-oxazol-5-ylmethyl]-3-[(naphthalene-1-carbonyl)-amino]-1-azonia-bicyclo[2.2.2]octane; formate

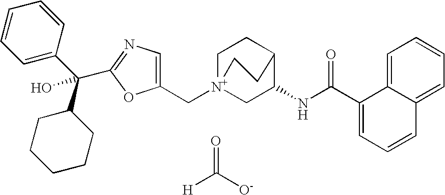

Step 1. N—(S)-1-aza-bicyclo[2.2.2]oct-3-yl-1-naphthyl-benzamide was prepared from 1-naphthoyl chloride and 3-S-aminoquinuclidine dihydrochloride by the procedure described in Procedure D. Data for N—(S)-1-aza-bicyclo[2.2.2]oct-3-yl-1-naphthyl-benzamide: LCMS (Method 7, Rt 1.89 min). MH⁺=281.

Step 2. The title compound was prepared from the foregoing compound and Intermediate 14 by application of General Procedure F. Data for the title compound: LCMS (Method 6, Rt 8.12 min). M⁺=550. ¹H NMR (400 MHz, CD₃OD): δ 8.50 (s, 1H), 8.20-8.16 (m, 1H), 8.01 (d, 1H), 7.96-7.92 (m, 1H), 7.65 (dd, 1H), 7.60-7.49 (m, 6H), 7.48 (s, 1H), 7.32 (t, 2H), 7.22 (t, 1H), 4.62 (s, 2H), 4.57 (s, 2H), 4.08-3.97 (m; 1H), 3.50-3.42 (m, 4H), 3.37-3.33 (m, 1H), 2.46-2.37 (m, 2H), 2.26 (s, 1H), 2.19-2.11 (m, 2H), 2.04-1.95 (m, 1H), 1.79-1.51 (m, 3H), 1.40-1.21 (m, 4H), 1.20-1.05 (m, 3H).

### Example 19

### (S)-3-(4-chloro-benzoylamino)-1-[2-((R)-cyclohexyl-hydroxy-phenyl-methyl)-oxazol-5-ylmethyl]-1-azonia-bicyclo[2.2.2]octane; formate

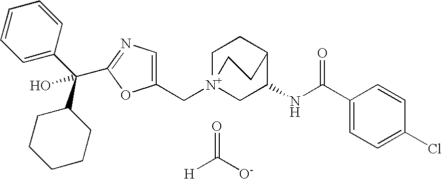

Step 1. N—(S)-1-aza-bicyclo[2.2.2]oct-3-yl-4-chloromethyl-benzamide was prepared from 4-chlorobenzoyl chloride and 3-S-aminoquinuclidine dihydrochloride by the procedure described in Procedure D. Data for N—(S)-1-aza-bicyclo[2.2.2]oct-3-yl-4-chloromethyl-benzamide: LCMS (Method 7, Rt 1.91 min). MH⁺=265.

Step 2. The title compound was prepared from the foregoing compound and Intermediate 14 by application of General Procedure F. Data for the title compound: LCMS (Method 6, Rt 7.91 min). M⁺=534. ¹H NMR (400 MHz, CD₃OD): δ 8.37 (s, 2H), 7.86-7.82 (m, 2H), 7.55-7.50 (m, 2H), 7.49 (dd, 3H), 7.34-7.29 (m, 2H), 7.24-7.19 (m, 1H), 4.60 (s, 2H), 4.42 (s, 1H), 3.95 (ddd, 1H), 3.53-3.38 (m, 4H), 3.36-3.31 (m, 1H), 2.44-2.34 (m, 2H), 2.28 (s, 1H), 2.15-2.08 (m, 2H), 2.03-1.93 (m, 1H), 1.77-1.50 (m, 4H), 1.40-1.21 (m, 3H), 1.23-1.03 (m, 4H).

### Example 20

### (R)-1-[2-((R)-cyclohexyl-hydroxy-phenyl-methyl)-oxazol-5-ylmethyl]-3-(4-trifluoromethoxy-benzyloxy)-1-azonia-bicyclo[2.2.2]octane; formate

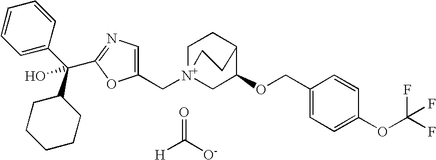

Step 1. (R)-3-(4-Trifluoromethoxy-benzyloxy)-1-aza-bicyclo[2.2.2]octane was prepared from 4-trifluoromethoxybenzyl bromide and 3-R-quinuclidinol by the procedure described in Procedure A. Data for (R)-3-(4-trifluoromethoxy-benzyloxy)-1-aza-bicyclo[2.2.2]octane: LCMS (Method 7, Rt 0.34 min). MH⁺=302.

Step 2. The title compound was prepared from the foregoing compound and Intermediate 14 by application of General Procedure F. Data for the title compound: LCMS (Method 6, 9.10 min). M⁺=no ion detected. ¹H NMR (400 MHz, CDCl₃): δ 8.62 (s, 1H), 7.55 (d, 2H), 7.37 (s, 1H), 7.29 (d, 2H), 7.28-7.11 (m, 6H), 5.14 (s, 4H), 4.91-4.56 (m, 2H), 4.44 (s, 2H), 3.97 (s, 1H), 3.58 (s, 2H), 3.56-3.37 (m, 3H), 3.18-3.04 (m, 2H), 2.26 (s, 2H), 2.21-1.93 (m, 1H), 1.87 (s, 2H), 1.31-1.14 (m, 3H), 1.16-1.03 (m, 3H).

### Example 21

### (R)-3-(4-cyano-benzyloxy)-1-[2-((R)-cyclohexyl-hydroxy-phenyl-methyl)-oxazol-5-ylmethyl]-1-azonia-bicyclo[2.2.2]octane; formate

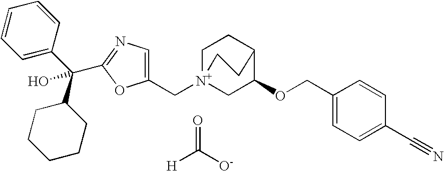

Step 1. (R)-3-(4-cyanobenzyloxy)-1-aza-bicyclo[2.2.2]octane was prepared from 4-cyanobenzyl bromide and 3-R-quinuclidinol by the procedure described in Procedure A. Data for (R)-3-(4-cyano-benzyloxy)-1-aza-bicyclo[2.2.2]octane: LCMS (Method 7, Rt 0.35 min). MH⁺=243.

Step 2. The title compound was prepared from the foregoing compound and Intermediate 14 by application of General Procedure F. Data for the title compound: LCMS (Method 6, Rt 7.97 min). M⁺=512. ¹H NMR (400 MHz, CDCl₃): δ 8.64 (s, 1H), 7.63 (d, 2H), 7.56 (d, 2H), 7.38 (d, 3H), 7.27 (d, 1H), 7.27-7.13 (m, 3H), 4.96-4.55 (m, 2H), 4.61-4.45 (m, 2H), 4.30 (s, 4H), 4.03 (d, 1H), 3.96 (s, 1H), 3.62 (s, 1H), 3.48 (t, 1H), 3.38 (s, 1H), 3.25-3.08 (m, 2H), 2.28 (s, 2H), 2.10 (d, 1H), 1.88 (s, 2H), 1.77 (s, 2H), 1.25 (d, 3H), 1.20-1.03 (m, 2H).

### Example 22

### (R)-1-[2-((R)-cyclohexyl-hydroxy-phenyl-methyl)-oxazol-5-ylmethyl]-3-(3,4-dichloro-benzyloxy)-1-azonia-bicyclo[2.2.2]octane; formate

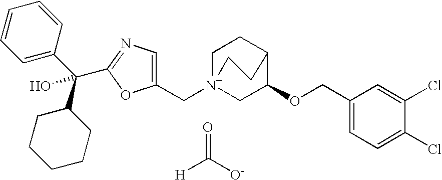

Step 1. (R)-3-(3,4-dichloro-benzyloxy)-1-aza-bicyclo[2.2.2]octane was prepared from 3,4-dichlorobenzyl bromide and 3-R-quinuclidinol by the procedure described in Procedure A. Data for (R)-3-(3,4-dichloro-benzyloxy)-1-aza-bicyclo[2.2.2]octane: LCMS (Method 7, Rt 2.25 min). MH⁺=286.

Step 2. The title compound was prepared from the foregoing compound and Intermediate 14 by application of General Procedure F. Data for the title compound: LCMS (Method 6, Rt 9.11 min). M⁺=555. ¹H NMR (400 MHz, CDCl₃): δ 8.60 (s, 1H), 7.54 (d, 2H), 7.42-7.34 (m, 3H), 7.24 (d, 2H), 7.17 (t, 1H), 7.11 (dd, 1H), 4.72 (s, 2H), 4.45-4.30 (m, 2H), 4.15 (s, 5H), 3.92 (s, 2H), 3.55 (s, 1H), 3.41 (d, 2H), 3.17-3.02 (m, 2H), 2.25 (d, 2H), 2.09-1.95 (m, 1H), 1.86 (s, 1H), 1.74 (s, 2H), 1.25 (d, 3H), 1.20-0.99 (m, 3H).

### Example 23

### (R)-3-(4-Chloro-benzyloxy)-1-[2-((R)-cyclohexyl-hydroxy-phenyl-methyl)-oxazol-5-ylmethyl]-1-azonia-bicyclo[2.2.2]octane; bromide

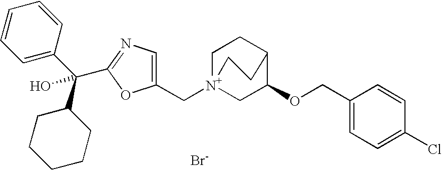

Step 1. (R)-3-(4-chloro-benzyloxy)-1-aza-bicyclo[2.2.2]octane was prepared from 4-chlorobenzyl bromide and 3-R-quinuclidinol by the procedure described in Procedure A. Data for (R)-3-(4-chloro-benzyloxy)-1-aza-bicyclo[2.2.2]octane: LCMS (Method 7, 0.37 min). MH⁺=252.

Step 2. The title compound was prepared from the foregoing compound and Intermediate 14 by application of General Procedure F. Data for the title compound: LCMS (Method 6, Rt 8.73 min). M⁺=521. ¹H NMR (400 MHz, CDCl₃): δ 7.58-7.53 (m, 2H), 7.48 (s, 1H), 7.34-7.28 (m, 2H), 7.30-7.22 (m, 2H), 7.21-7.14 (m, 3H), 5.17-4.97 (m, 2H), 4.42 (s, 2H), 4.37-4.23 (m, 2H), 4.08-3.95 (m, 2H), 3.81-3.66 (m, 2H), 3.27-3.15 (m, 2H), 2.32 (d, 2H), 2.18-2.08 (m, 1H), 2.06-1.80 (m, 3H), 1.72-1.61 (m, 3H), 1.37-1.21 (m, 4H), 1.22-1.03 (m, 3H).

### Example 24

### (S)-1-[2-((R)—Cyclohexyl-hydroxy-phenyl-methyl)-oxazol-5-ylmethyl]-3-phenoxymethyl-1-azonia-bicyclo[2.2.2]octane; bromide

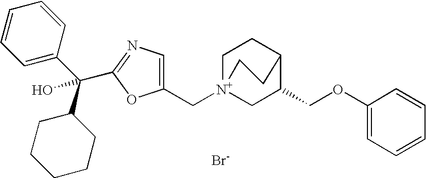

Method 1. Step 1. A solution of (1-aza-bicyclo[2.2.2]oct-3-yl)-methanol (Heterocycles 1987, 25(1), 251-8) (982 mg), iodobenzene (0.78 mL), copper iodide (133 mg), 1,10-phenanthroline (251 mg) and Cs₂CO₃ (4.53 g) in toluene (1.75 mL) was heated at 110° C. for 2 days. The reaction mixture was cooled to room temperature, filtered through Celite, and the residue was washed with DCM. The combined organic phases were evaporated in vacuo, re-dissolved in DCM and washed with water and copper sulfate solution, dried (MgSO₄), filtered and evaporated in vacuo. Purification by SCX gave the desired compound (509 mg, 34%) as an off-white solid. A portion of this compound (0.2 g) was separated into its enantiomers by use of HPLC Method 3, to give Enantiomer 1 (S)-configuration (Retention time=14.16 min) (58.4 mg) and Enantiomer 2 (R)-configuration (Retention time=15.86 min) (56.8 mg), both as colourless oils. Data for 3R-phenoxymethyl-1-aza-bicyclo[2.2.2]octane: LCMS (Method 7, 0.35 min). MH⁺=218.

Step 2. The title compound was prepared from the foregoing compound (Enantiomer 1) and Intermediate 14 by application of General Procedure F. Data for the title compound: LCMS (Method 6, 8.40 min). M⁺=487.33. ¹H NMR (400 MHz, CH₃OH-d₄): δ 7.54-7.49 (m, 2H), 7.47 (s, 1H), 7.34-7.19 (m, 5H), 6.98-6.87 (m, 3H), 4.59 (s, 2H), 4.05-3.99 (m, 2H), 3.69 (t, 1H), 3.47 (d, 4H), 3.41 (s, 1H), 3.20 (dd, 1H), 2.72-2.62 (m, 1H), 2.45-2.37 (m, 1H), 2.27 (d, 1H), 2.19 (s, 1H), 2.07 (t, 2H), 1.98-1.88 (m, 1H), 1.78-1.52 (m, 4H), 1.33-1.04 (m, 6H).

Method 2. The title compound may also be prepared by the following method.

Step 1. A suspension of sodium hydride (380 mg, 60% dispersion in mineral oil) in DMF (5 mL) was treated carefully with phenol (0.9 g). After 10 min, the reaction was treated with methanesulfonic acid (S)-1-(1-aza-bicyclo[2.2.2]oct-3-yl)methyl ester (L)-tartrate salt (0.5 g) (Y. Guminski et al., Org. Prep. Proc. Int., 1999, 31, 399) and the reaction mixture was heated at 100° C. for 1 h. The reaction mixture was cooled, and diluted with water and ethyl acetate. The organic phase was separated and washed with 1M NaOH and brine, dried (MgSO₄), filtered and evaporated in vacuo. Purification by SCX and silica gel chromatography gave 3S-phenoxymethyl-1-aza-bicyclo[2.2.2]octane (0.19 g, 65%).

Step 2. The title compound was prepared from the foregoing compound and Intermediate 14 by application of General Procedure F. Data for the title compound: LCMS (Method 6, Rt 8.40 min). M⁺=487.

### Example 25

### (R)-1-[2-((R)—Cyclohexyl-hydroxy-phenyl-methyl)-oxazol-5-ylmethyl]-3-phenoxymethyl-1-azonia-bicyclo[2.2.2]octane; bromide

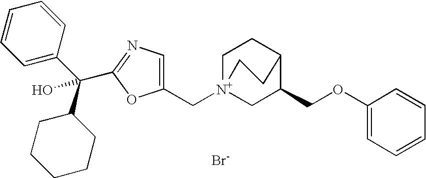

The title compound was prepared from the compound of Step 1 Example 24 (Enantiomer 2) and Intermediate 14 by application of General Procedure F. Data for the title compound: LCMS (Method 6, 8.42 min). M⁺=487. ¹H NMR (400 MHz, CD₃OD): δ 7.56-7.52 (m, 2H), 7.50-7.45 (m, 1H), 7.33-7.19 (m, 5H), 6.98-6.90 (m, 3H), 4.72-4.51 (m, 2H), 4.10-3.99 (m, 2H), 3.70 (ddd, 1H), 3.53-3.38 (m, 5H), 3.21 (ddd, 1H), 2.73-2.63 (m, 1H), 2.42 (t, 1H), 2.28 (d, 1H), 2.11-2.04 (m, 2H), 1.99-1.89 (m, 1H), 1.79-1.60 (m, 3H), 1.56 (d, 1H), 1.42-1.28 (m, 4H), 1.29-1.05 (m, 3H).

### Example 26

### (R)-1-[2-(Hydroxy-diphenyl-methyl)-oxazol-5-ylmethyl]-3-phenoxymethyl-1-azonia-bicyclo[2.2.2]octane; bromide

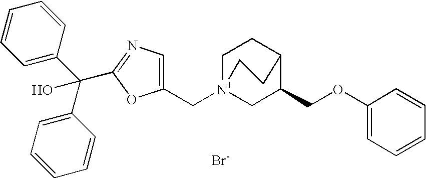

The title compound was prepared from the compound of Step 1 Example 24 (Enantiomer 2) and Intermediate 8 by application of General Procedure F. Data for the title compound: LCMS (Method 6, Rt 7.81 min). M⁺=481. ¹H NMR (400 MHz, CD₃OD): δ 7.51 (s, 1H), 7.37-7.25 (m, 12H), 6.97-6.89 (m, 3H), 4.63 (s, 2H), 4.08-3.98 (m, 2H), 3.68 (ddd, 1H), 3.51-3.36 (m, 5H), 3.20 (ddd, 1H), 2.66 (q, 1H), 2.27 (d, 1H), 2.21-2.14 (m, 1H), 2.13-2.03 (m, 2H), 1.97-1.87 (m, 1H).

### Example 27

### (R)-3-Benzyloxy-1-[2-(hydroxy-diphenyl-methyl)-oxazol-5-ylmethyl]-1-azonia-bicyclo[2.2.2]octane; bromide

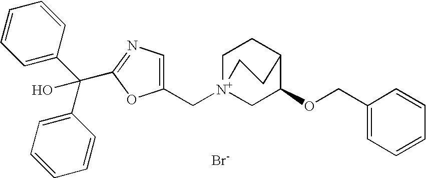

The title compound was prepared from the compound of Step 1 Example 12 and Intermediate 8 by application of General Procedure F. Data for the title compound: LCMS (Method 6, Rt 7.61 min). M⁺=481. ¹H NMR (400 MHz, CDCl₃): δ 7.49 (s, 1H), 7.38-7.29 (m, 7H), 7.31-7.25 (m, 3H), 7.26-7.19 (m, 5H), 5.74 (s, 1H), 4.73 (s, 2H), 4.48-4.38 (m, 2H), 4.19-4.11 (m, 1H), 3.93 (s, 1H), 3.88-3.77 (m, 1H), 3.74-3.64 (m, 2H), 3.15-3.05 (m, 1H), 3.00 (d, 1H), 2.29 (s, 1H), 2.16-2.05 (m, 1H), 1.97-1.88 (m, 1H), 1.83 (d, 2H).

### Example 28

### (R)-3-(4-Chloro-benzyloxy-1-[2-(hydroxy-diphenyl-methyl)-oxazol-5-ylmethyl]-1-azonia-bicyclo[2.2.2]octane; bromide

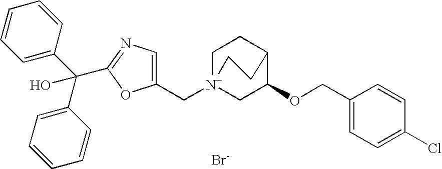

The title compound was prepared from the compound of Step 1 Example 23 and Intermediate 8 by application of General Procedure F. Data for the title compound: LCMS (Method 6, Rt 8.11 min). M⁺=515. ¹H NMR (400 MHz, DMSO-d₆): δ 7.52 (s, 1H), 7.46-7.42 (m, 2H), 7.39-7.35 (m, 6H), 7.34-7.24 (m, 6H), 7.13 (s, 1H), 4.72-4.61 (m, 2H), 4.57-4.43 (m, 2H), 4.01-3.95 (m, 1H), 3.70-3.61 (m, 1H), 3.47-3.31 (m, 2H), 3.31-3.20 (m; 3H), 2.42 (s, 1H), 2.08-1.92 (m, 2H), 1.78 (s, 2H).

### Example 29

### (R)-3-Benzyloxy-1-[2-(cyclohexyl-hydroxy-phenyl-methyl)-thiazol-5-ylmethyl]-1-azonia-bicyclo[2.2.2]octane; bromide

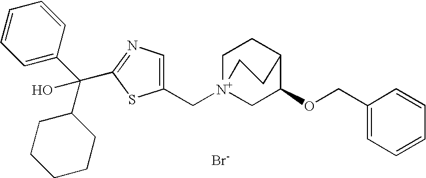

The title compound was prepared from the compound of Step 1 Example 12 and Intermediate 19 by application of General Procedure F. Data for the title compound: LCMS (Method 6, Rt 8.62 min). M⁺=503. ¹H NMR (400 MHz, DMSO-d₆): δ 7.92 (d, 1H), 7.70-7.65 (m, 2H), 7.36-7.25 (m, 7H), 7.20 (t, 1H), 6.30 (d, 1H), 4.74-4.64 (m, 2H), 4.56-4.42 (m, 2H), 3.97 (s, 1H), 3.68-3.56 (m, 1H), 3.44-3.33 (m, 3H), 2.38 (s, 2H), 2.04 (s, 2H), 1.98-1.88 (m, 1H), 1.84-1.70 (m, 3H), 1.68-1.52 (m, 4H), 1.35-1.18 (m, 3H), 1.21-1.04 (m, 3H).

### Example 30

### (R)-3-benzyloxy-1-[3-(cyclohexyl-hydroxy-phenyl-methyl)-[1,2,4]oxadiazol-5-ylmethyl]-1-azonia-bicyclo[2.2.2]octane; formate

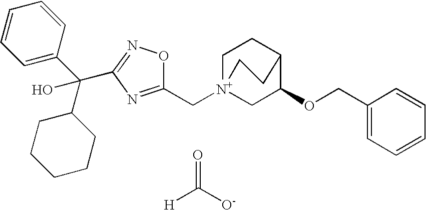

The title compound was prepared from the compound of Step 1 Example 12 and Intermediate 20, Enantiomer 2 by application of General Procedure F. Data for the title compound: LCMS (Method 6, Rt 8.56 min). M⁺=488. ¹H NMR (400 MHz, CDCl₃): δ 8.54 (s, 1H), 7.42-7.38 (m, 2H), 7.37-7.30 (m, 2H), 7.32-7.27 (m, 3H), 7.27 (s, 1H), 7.25-7.13 (m, 2H), 5.18 (d, 1H), 4.76 (d, 1H), 4.57-4.43 (m, 2H), 4.15-4.05 (m, 1H), 4.01 (s, 1H), 3.90 (s, 5H), 3.82 (d, 4H), 3.39 (t, 2H), 2.36 (s, 1H), 2.23 (d, 2H), 1.90 (s, 2H), 1.27 (d, 3H), 1.22-1.06 (m, 3H).

### Example 31

### (R)-3-benzyloxy-1-[3-(cyclohexyl-hydroxy-phenyl-methyl)-isoxazol-5-ylmethyl]-1-azonia-bicyclo[2.2.2]octane; formate

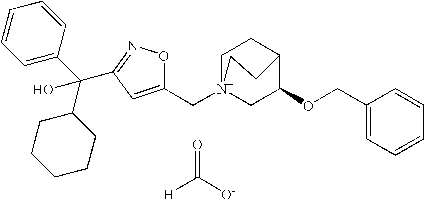

The title compound was prepared from the compound of Step 1 Example 12 and Intermediate 21, Enantiomer 2 by application of General Procedure F. Data for the title compound: LCMS (Method 6, Rt 8.69 min). M⁺=487. ¹H NMR (400 MHz, CDCl₃): δ 8.64 (s, 1H), 7.58-7.51 (m, 2H), 7.38-7.35 (t, 1H), 7.32-7.23 (m, 5H), 7.20 (t, 1H), 7.38-7.35 (t, 1H), 7-7.11 (s, 1H), 5.04-4.85 (m, 2H), 4.54-4.46 (m, 2H), 4.22-4.10 (m, 1H), 3.98 (s, 1H), 3.82 (s, 1H), 3.73 (t, 1H), 3.52 (s, 1H), 3.39-3.28 (m, 1H), 3.26 (d, 1H), 2.43 (d, 3H), 2.25 (s, 3H), 1.98 (s, 1H), 1.85 (s, 3H), 1.73 (s, 1H), 1.32-1.22 (m, 3H), 1.20-1.05 (m, 3H).

### Example 32

### (R)-1-[2-((R)—Cyclohexyl-hydroxy-phenyl-methyl)-oxazol-5-ylmethyl]-3-(4-fluoro-benzyloxy)-1-azonia-bicyclo[2.2.2]octane; bromide

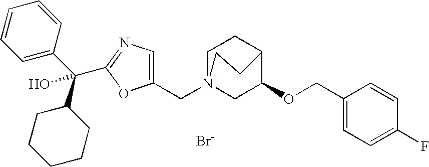

Step 1. (R)-3-(4-fluoro-benzyloxy)-1-aza-bicyclo[2.2.2]octane was prepared from 4-fluorobenzyl bromide and 3-R-quinuclidinol by the procedure described in Procedure A. Data for (R)-3-(4-fluoro-benzyloxy)-1-aza-bicyclo[2.2.2]octane: LCMS (Method 7, Rt 0.36 min). MH⁺=236.

Step 2. The title compound was prepared from the foregoing compound and Intermediate 14 by application of General Procedure F. Data for the title compound: LCMS (Method 6, Rt 8.36 min). M⁺=505. ¹H NMR (400 MHz, CDCl₃): δ 7.56 (d, 2H), 7.48 (s, 1H), 7.28 (s, 1H), 7.28-7.16 (m, 4H), 7.03 (t, 2H), 5.16-4.98 (m, 2H), 4.41 (s, 2H), 4.28 (s, 3H), 3.99 (s, 2H), 3.81-3.69 (m, 2H), 3.18 (d, 2H), 2.34 (s, 2H), 2.29 (s, 1H), 2.13 (s, 1H), 2.05-1.94 (m, 1H), 1.94-1.79 (m, 3H), 1.34-1.17 (m, 4H), 1.22-1.01 (m, 3H).

### Example 33

### (R)-1-[2-((R)—Cyclohexyl-hydroxy-phenyl-methyl)-oxazol-5-ylmethyl]-3-(4-trifluoromethyl-benzyloxy)-1-azonia-bicyclo[2.2.2]octane; bromide

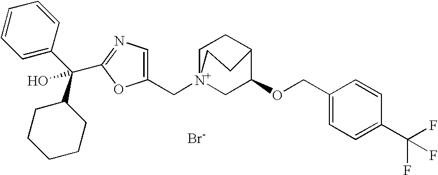

Step 1. (R)-3-(4-trifluoromethyl-benzyloxy)-1-aza-bicyclo[2.2.2]octane was prepared from 4-fluorobenzyl bromide and 3-R-quinuclidinol by the procedure described in Procedure A. Data for (R)-3-(4-trifluoromethyl-benzyloxy)-1-aza-bicyclo[2.2.2]octane: LCMS (Method 7, Rt 0.36 min). MH⁺=286.

Step 2. The title compound was prepared from the foregoing compound and Intermediate 14 by application of General Procedure F. Data for the title compound: LCMS (Method 6, Rt 8.96 min). M⁺=555. ¹H NMR (400 MHz, CDCl₃): δ 7.62-7.54 (m, 4H), 7.49 (s, 1H), 7.39 (d, 2H), 7.29-7.22 (m, 2H), 7.16 (t, 1H), 5.17-5.00 (m, 2H), 4.56-4.47 (m, 2H), 4.37-4.27 (m, 2H), 4.06-3.95 (m, 2H), 3.82-3.64 (m, 2H), 3.31-3.20 (m, 2H), 2.38 (s, 1H), 2.30 (s, 1H), 2.24-2.05 (m, 1H), 2.05-1.94 (m, 1H), 1.82 (s, 5H), 1.36-1.20 (m, 4H), 1.19-1.03 (m, 3H).

### Example 34

### (R)-1-[2-((R)—Cyclohexyl-hydroxy-phenyl-methyl)-oxazol-5-ylmethyl]-3-(4-methyl-benzyloxy)-1-azonia-bicyclo[2.2.2]octane; bromide

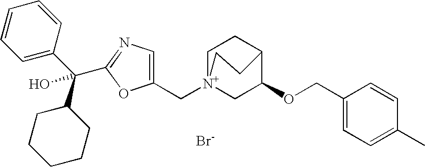

Step 1. (R)-3-(4-methyl-benzyloxy)-1-aza-bicyclo[2.2.2]octane was prepared from 4-methylbenzyl bromide and 3-R-quinuclidinol by the procedure described in Procedure A. Data for (R)-3-(4-methyl-benzyloxy)-1-aza-bicyclo[2.2.2]octane: LCMS (Method 7, Rt 0.37 min). MH⁺=232.

Step 2. The title compound was prepared from the foregoing compound and Intermediate 14 by application of General Procedure F. Data for the title compound: LCMS (Method 6, Rt 8.76 min). M⁺=501. ¹H NMR (400 MHz, CDCl₃): δ 7.55 (d, 2H), 7.46 (s, 1H), 7.32-7.20 (m, 2H), 7.20-7.11 (m, 5H), 5.15-4.95 (m, 2H), 4.45-4.35 (m, 2H), 4.22 (s, 2H), 3.96 (s, 2H), 3.77 (s, 3H), 3.18-3.06 (m, 2H), 2.34 (s, 4H), 2.12 (s, 1H), 1.97 (d, 1H), 1.90-1.75 (m, 5H); 1.29 (d, 4H), 1.23-1.03 (m, 3H).

### Example 35

### (R)-1-[2-((R)-cyclohexyl-hydroxy-phenyl-methyl)-oxazol-5-ylmethyl]-3-(3-fluoro-benzyloxy)-1-azonia-bicyclo[2.2.2]octane; formate

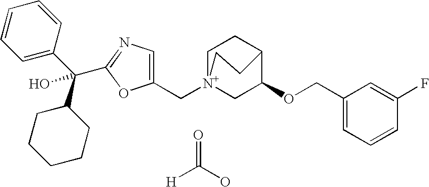

Step 1. (R)-3-(3-fluoro-benzyloxy)-1-aza-bicyclo[2.2.2]octane was prepared from 3-fluorobenzyl bromide and 3-R-quinuclidinol by the procedure described in Procedure A. Data for (R)-3-(3-fluoro-benzyloxy)-1-aza-bicyclo[2.2.2]octane: LCMS (Method 7, Rt 0.36 min). MH⁺=236.

Step 2. The title compound was prepared from the foregoing compound and Intermediate 14 by application of General Procedure F. Data for the title compound: LCMS (Method 6, Rt 8.45 min). M⁺=505. ¹H NMR (400 MHz, CDCl₃): δ 8.66 (s, 1H), 7.58-7.52 (m, 2H), 7.36 (s, 1H), 7.31 (td, 1H), 7.26-7.13 (m, 3H), 7.07-6.95 (m, 3H), 5.83 (s, 2H), 4.83-4.66 (m, 2H), 4.44 (s, 2H), 4.06-3.96 (m, 1H), 3.92 (s, 1H), 3.67-3.47 (m, 2H), 3.44 (d, 1H), 3.11 (d, 2H), 2.27 (s, 2H), 2.14-2.04 (m, 1H), 1.87 (s, 1H), 1.75 (d, 2H), 1.72-1.54 (m, 3H), 1.42-1.14 (m, 3H), 1.18-1.02 (m, 3H).

### Example 36

### 1-[2-((R)—Cyclohexyl-hydroxy-phenyl-methyl)-oxazol-5-ylmethyl]-3-phenethyl-1-azonia-bicyclo[2.2.2]octane; bromide

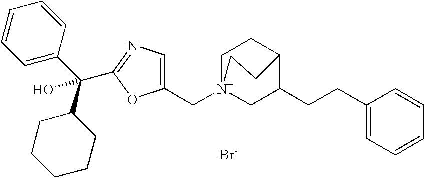

Step 1. (RS)-3-phenethyl-1-aza-bicyclo[2.2.2]octane was prepared by the procedure described in Procedure E.

Step 2. The title compound was prepared from the foregoing compound and Intermediate 14 by application of General Procedure F. Data for the title compound: LCMS (Method 6, Rt 8.68 min). M⁺=485. ¹H NMR (400 MHz, DMSO-d₆): δ 7.45 (d, 3H), 7.32-7.25 (m, 4H), 7.24-7.16 (m, 4H), 6.06 (d, 1H), 4.59-4.48 (m, 2H), 3.56-3.44 (m, 1H), 3.33 (s, 4H), 2.87 (dt, 1H), 2.54-2.50 (m, 2H), 2.25 (t, 1H), 1.98 (s, 3H), 1.94-1.71 (m, 3H), 1.70-1.54 (m, 6H), 1.27 (d, 1H), 1.18 (t, 2H), 1.13-0.90 (m, 3H).

### Example 37

### 1-[2-(Hydroxy-diphenyl-methyl)-oxazol-5-ylmethyl]-3-phenethyl-1-azonia-bicyclo[2.2.2]octane; bromide

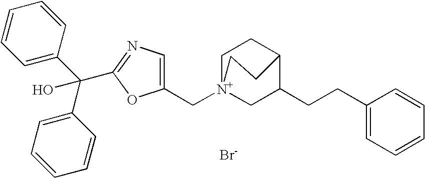

The title compound was prepared from the compound of Step 1 Example 36 and Intermediate 8 by application of General Procedure F. Data for the title compound: LCMS (Method 6, Rt 7.98 min). M⁺=479. ¹H NMR (400 MHz, DMSO-d₆): δ 7.52 (s, 1H), 7.39-7.36 (m, 4H), 7.34-7.26 (m, 7H), 7.27-7.17 (m, 4H), 7.11 (s, 1H), 4.65-4.50 (m, 2H), 3.59-3.47 (m, 1H), 2.93 (dd, 1H), 2.56-2.52 (m, 1H), 2.01 (d, 4H), 1.95-1.87 (m, 1H), 1.88-1.72 (m, 2H), 1.72-1.62 (m, 2H), 1.40 (s, 4H).

### Example 38

### (R)-1-[2-((R)-cyclohexyl-hydroxy-phenyl-methyl)-oxazol-5-ylmethyl]-3-(3-methyl-but-2-enyloxy)-1-azonia-bicyclo[2.2.2]octane; formate

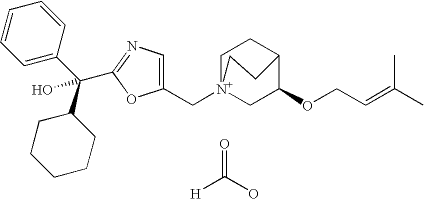

Step 1. (R)-3-(3-Methyl-but-2-enyloxy)-1-aza-bicyclo[2.2.2]octane was prepared from prenyl bromide and 3-R-quinuclidinol by the procedure described in Procedure A. Data for (R)-3-(3-Methyl-but-2-enyloxy)-1-aza-bicyclo[2.2.2]octane: LCMS (Method 7, 0.37 min). MH⁺=196.

Step 2. The title compound was prepared from the foregoing compound and Intermediate 14 by application of General Procedure F. Data for the title compound: LCMS (Method 6, Rt 8.15 min). M⁺=465. ¹H NMR (400 MHz, CDCl₃): δ 8.67 (s, 1H), 7.58-7.53 (m, 2H), 7.36 (d, 1H), 7.32-7.26 (m, 2H), 7.24-7.15 (m, 1H), 5.25-5.20 (m, 1H), 4.91 (s, 3H), 4.81-4.66 (m, 2H), 3.99-3.79 (m, 3H), 3.62-3.39 (m, 3H), 3.09-2.95 (m, 2H), 2.32-2.16 (m, 2H), 2.08-1.96 (m, 1H), 1.87 (d, 1H), 1.81-1.65 (m, 6H), 1.64 (s, 3H), 1.57 (t, 2H), 1.27 (d, 3H), 1.23-1.02 (m, 3H).

### Example 39

### 3-Benzylsulfanyl-1-[2-(hydroxy-diphenyl-methyl)-oxazol-5-ylmethyl]-1-azonia-bicyclo[2.2.2]octane; bromide,

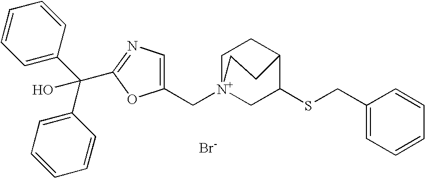

Step 1. (RS)-(3-Benzylsulfanyl-1-aza-bicyclo[2.2.2]octane was prepared from benzyl mercaptan and 3-chloroquinuclidine hydrochloride by the procedure described in Procedure C1. Data for (RS)-(3-benzylsulfanyl-1-aza-bicyclo[2.2.2]octane: LCMS (Method 7, Rt 2.12 min). MH⁺=234.

Step 2. The title compound was prepared from the foregoing compound and Intermediate 8 by application of General Procedure F. Data for the title compound: LCMS (Method 6, Rt 8.14 min). M⁺=497. ¹H NMR (400 MHz, DMSO-d₆): δ 7.49 (s, 1H), 7.39-7.35 (m, 4H), 7.36-7.34 (m, 5H), 7.33-7.30 (m, 3H), 7.29-7.24 (m, 3H), 7.12 (s, 1H), 4.61 (s, 2H), 3.87-3.78 (m, 2H), 3.78-3.70 (m, 1H), 3.44-3.36 (m, 3H), 3.31-3.23 (m, 2H), 3.06 (ddd, 1H), 2.16 (d, 2H), 2.06-1.96 (m, 1H), 1.84 (d, 2H).

### Example 40

### (S)-3-Benzylsulfanyl-1-[2-((R)-cyclohexyl-hydroxy-phenyl-methyl)-oxazol-5-ylmethyl]-1-azonia-bicyclo[2.2.2]octane; bromide

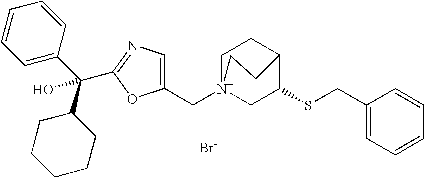

Step 1. (S)-(3-Benzylsulfanyl-1-aza-bicyclo[2.2.2]octane was prepared from methanesulfonic acid (R)-(1-aza-bicyclo[2.2.2]oct-3-yl0 ester and benzyl mercaptan by analogy to the procedure described in Procedure C2.

Step 2. The title compound was prepared from the foregoing compound and Intermediate 14 by application of General Procedure F. Data for the title compound: LCMS (Method 6, Rt 8.76 min). M⁺=503. ¹H NMR (400 MHz, CDCl₃): δ 7.53 (d, 2H), 7.38 (s, 1H), 7.37-7.19 (m, 4H), 7.21 (dd, 4H), 5.12-4.92 (m, 2H), 4.14 (t, 1H), 3.83 (s, 2H), 3.75-3.71 (m, 1H), 3.68 (s, 2H), 3.33-3.19 (m, 1H), 3.15 (s, 1H), 2.86 (dd, 1H), 2.32-2.17 (m, 2H), 2.00 (d, 2H), 1.84 (d, 4H), 1.72 (d, 2H), 1.34 (d, 1H), 1.31-1.16 (m, 3H), 1.20-1.08 (m, 3H).

### Example 41

### (R)-3-Benzylsulfanyl-1-[2-((R)-cyclohexyl-hydroxy-phenyl-methyl)-oxazol-5-ylmethyl]-1-azonia-bicyclo[2.2.2]octane; bromide

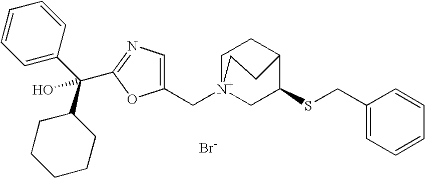

Step 1. (R)-(3-Benzylsulfanyl-1-aza-bicyclo[2.2.2]octane was prepared by the procedure described in Procedure C2.

Step 2. The title compound was prepared from the foregoing compound and Intermediate 14 by application of General Procedure F. Data for the title compound: LCMS (Method 6, Rt 8.79 min). M⁺=503. ¹H NMR (400 MHz, CD₃OD): δ 7.54-7.50 (m, 2H), 7.37-7.23 (m, 9H), 4.49 (s, 2H), 3.83 (s, 2H), 3.62 (d, 1H), 3.41-3.32 (m, 5H), 3.29-3.20 (m, 3H), 3.03-2.97 (m, 1H), 2.40 (s, 2H), 2.15 (d, 1H), 1.90 (d, 2H), 1.68 (s, 2H), 1.54 (s, 1H), 1.33 (dd, 3H), 1.21-1.10 (m, 3H).

### Example 42

### (R)-3-benzyloxy-1-[2-((R)-cyclopentyl-hydroxy-phenyl-methyl)-oxazol-5-ylmethyl]-1-azonia-bicyclo[2.2.2]octane; formate

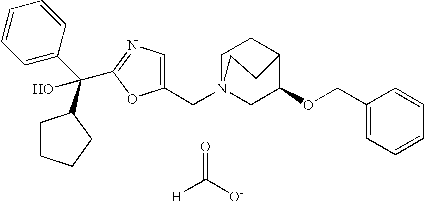

The title compound was prepared from the compound of Step 1 Example 12 and Intermediate 16 by application of General Procedure F. Data for the title compound: LCMS (Method 6, Rt 8.04 min). M⁺=473. ¹H NMR (400 MHz, CDCl₃): δ 8.59 (s, 1H), 7.54-7.49 (m, 2H), 7.38-7.28 (m, 4H), 7.27-7.10 (m, 5H), 4.70 (s, 2H), 4.49-4.39 (m, 5H), 3.94-3.82 (m, 2H), 3.59-3.37 (m, 3H), 3.11-2.98 (m, 2H), 2.99-2.86 (m, 1H), 2.26 (s, 1H), 2.06 (t, 1H), 1.88 (s, 1H), 1.80-1.60 (m, 4H), 1.61-1.49 (m, 3H), 1.31-1.22 (m, 1H).

### Example 43

### (S)-1-[2-((R)—Cyclohexyl-hydroxy-phenyl-methyl)-oxazol-5-ylmethyl]-3-phenylsulfanylmethyl-1-azonia-bicyclo[2.2.2]octane; bromide

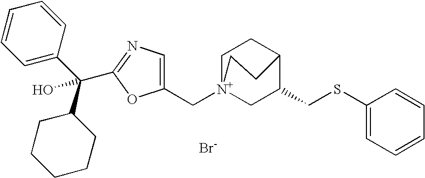

Step 1. Thiophenol (1 g) was added to a stirred suspension of sodium hydride (379 mg of 60% dispersion in mineral oil) in DMF (5 mL). After 10 min, methanesulfonic acid (S)-1-(1-aza-bicyclo[2.2.2]oct-3-yl)methyl ester (L)-tartrate salt (0.5 g) (Y. Guminski et al., Org. Prep. Proc. Int., 1999, 31, 399) was added and the reaction mixture was heated at 100° C. for 1 h. The reaction mixture was cooled, and diluted with water and ethyl acetate. The organic phase was separated and washed with 1M NaOH and, brine; dried (MgSO₄), filtered and evaporated in vacuo. Purification by SCX and silica gel chromatography (eluting with 0-10% [2M NH₃ in MeOH]-DCM) gave (S)-3-phenylsulfanylmethyl-1-aza-bicyclo[2.2.2]octane (200 mg, 53%) as a colourless oil. ¹H NMR (300 MHz, CDCl₃) 7.36-7.27 (4H, m), 7.21-7.15 (1H, m), 3.16-2.73 (7H, m), 2.51-2.43 (1H, m), 1.89-1.35 (6H, m).

Step 2. The title compound was prepared from the foregoing compound and Intermediate 14 by application of General Procedure F. Data for the title compound: LCMS (Method 6, Rt 8.81 min). M⁺=503. ¹H NMR (400 MHz, DMSO-d₆): δ 7.50-7.44 (m, 3H), 7.41-7.29 (m, 6H), 7.26-7.20 (m, 2H), 7.15 (s, 1H), 6.07 (s, 1H), 4.62 (d, 2H), 3.18-3.01 (m, 3H), 2.28-2.17 (m, 2H), 2.11 (s, 1H), 2.04 (d, 2H), 1.85 (d, 3H), 1.69 (s, 1H), 1.65-1.52 (m, 3H), 1.40 (s, 4H), 1.32-1.14 (m, 3H), 1.15-0.89 (m, 2H).

### Example 44

### (R)-3-(4-chloro-benzyloxy)-1-[2-((R)-cyclopentyl-hydroxy-phenyl-methyl)-oxazol-5-ylmethyl]-1-azonia-bicyclo[2.2.2]octane; formate

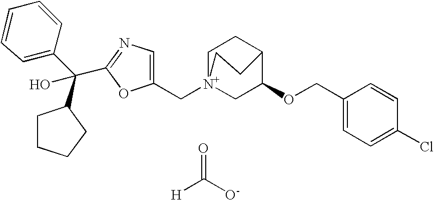

The title compound was prepared from the compound of Step 1 Example 23 and Intermediate 16 by application of General Procedure F. Data for the title compound: LCMS (Method 6, Rt 8.52 min). M⁺=507. ¹H NMR (400 MHz, CDCl₃): δ 8.57 (s, 1H), 7.54-7.49 (m, 2H), 7.36 (s, 1H), 7.31 (d, 2H), 7.26-7.12 (m, 5H), 4.94 (s, 4H), 4.70 (s, 2H), 4.40 (s, 2H), 3.90 (s, 2H), 3.58-3.34 (m, 3H), 3.14-3.02 (m, 2H), 2.96-2.85 (m, 1H), 2.25 (s, 1H), 2.05 (t, 1H), 2.01-1.74 (m, 1H), 1.74 (d, 2H), 1.77-1.51 (m, 2H), 1.47-1.38 (m, 2H), 1.32-1.22 (m, 1H).

### Example 45

### (R)-1-[2-((R)-cyclopentyl-hydroxy-phenyl-methyl)-oxazol-5-ylmethyl]-3-(3,4-dichloro-benzyloxy)-1-azonia-bicyclo[2.2.2]octane; formate

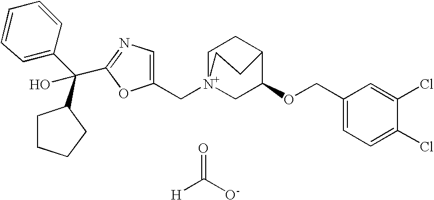

The title compound was prepared from the compound of Step 1 Example 22 and Intermediate 16 by application of General Procedure F. Data for the title compound: LCMS (Method 6, Rt 8.86 min). M⁺=541. ¹H NMR (400 MHz, CDCl₃): δ 8.56 (s, 1H), 7.54-7.49 (m, 2H), 7.43-7.31 (m, 3H), 7.24 (t, 2H), 7.16 (t, 1H), 7.11 (dd, 1H), 5.88 (s, 3H), 4.71 (s, 2H), 4.44-4.34 (m, 2H), 3.94 (d, 2H), 3.60-3.43 (m, 2H), 3.39 (d, 1H), 3.19-3.06 (m, 2H), 2.97-2.84 (m, 1H), 2.27 (s, 1H), 2.12-2.02 (m, 1H), 1.88 (s, 1H), 1.76 (s, 3H), 1.67-1.58 (m, 2H), 1.48-1.35 (m, 2H), 1.32-1.19 (m, 1H).

### Example 46

### (R)-1-[2-((A)-Cyclohexyl-hydroxy-phenyl-methyl)-oxazol-5-ylmethyl]-3-(thiophen-3-ylmethoxy)-1-azonia-bicyclo[2.2.2]octane; bromide

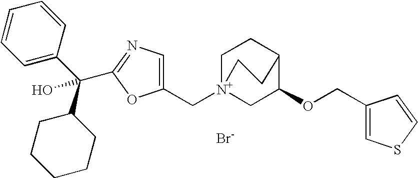

Step 1. (R)-3-(Thiophen-3-ylmethoxy)-1-aza-bicyclo[2.2.2]octane was prepared from 3-bromomethyl-thiophene (Tetrahedron, 2006, 62, 6182) and 3-R-quinuclidinol by the procedure described in Procedure A. Data for (R)-3-(thiophen-3-ylmethoxy)-1-aza-bicyclo[2.2.2]octane: LCMS (Method 7, Rt 0.37 min). MH⁺=224.

Step 2. The title compound was prepared from the foregoing compound and Intermediate 14 by application of General Procedure F. Data for the title compound: LCMS (Method 6, Rt 8.27 min). M⁺=493. ¹H NMR (400 MHz, CHCl₃-d): δ 7.59-7.54 (m, 2H), 7.47 (s, 1H), 7.34-7.26 (m, 2H), 7.27-7.25 (m, 1H), 7.22-7.17 (m, 2H), 6.99 (dd, 1H), 5.15-4.99 (m, 2H), 4.52-4.42 (m, 2H), 4.28-4.20 (m, 2H), 4.07-3.93 (m, 2H), 3.82-3.67 (m, 2H), 3.25-3.10 (m, 2H), 2.32 (s, 2H), 2.19-2.09 (m, 1H), 2.06-1.78 (m, 3H), 1.74 (d, 1H), 1.68 (d, 2H), 1.39-1.24 (m, 4H), 1.21-1.06 (m, 3H).

### Example 47

### 4-Benzyloxymethyl-1-[2-((R)-cyclohexyl-hydroxy-phenyl-methyl)-oxazol-5-ylmethyl]-1-azonia-bicyclo[2.2.2]octane; bromide

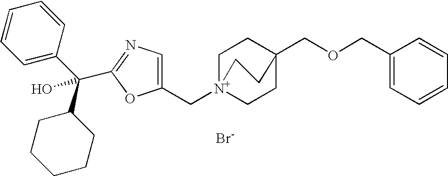

Step 1. A solution of (1-boranyl-1-aza-bicyclo[2.2.2]oct-4-yl)-methanol (Step 2, Example 55) (100 mg) in DMF (2 mL) was treated with sodium hydride (29 mg of 60% dispersion in mineral oil), stirred at room temperature for 15 min, then treated with benzyl bromide (0.086 mL). After 3 h at room temperature, the reaction mixture was quenched with water and extracted with ethyl acetate. The organic phase was washed with brine, dried (MgSO₄), filtered and evaporated in vacuo. The resulting product was dissolved in acetone, treated with excess 1.25 M HCl-MeOH, stirred for 0.5 h, evaporated in vacuo and purified by SCX to give 4-benzyloxymethyl-1-aza-bicyclo[2.2.2]octane (100 mg, 60%). ¹H NMR (300 MHz, CDCl₃) δ7.35-7.26 (5H, m), 4.49 (2H, s), 3.10 (2H, s), 2.91-2.85 (6H, m), 1.46-1.40 (6H, m).

Step 2. The title compound was prepared from the foregoing compound and Intermediate 14 by application of General Procedure F. Data for the title compound: LCMS (Method 6, Rt 8.62 min). M⁺=501. ¹H NMR (400 MHz, CDCl₃): δ 7.60-7.55 (m, 2H), 7.49 (s, 1H), 7.38-7.27 (m, 6H), 7.26-7.20 (m, 2H), 5.21-5.02 (m, 2H), 4.46 (s, 2H), 4.14 (s, 1H), 3.71 (d, 6H), 3.30 (t, 1H), 3.19 (s, 2H), 2.32 (d, 1H), 1.75 (d, 1H), 1.43 (s, 1H), 1.41-1.25 (m, 4H), 1.20-1.08 (m, 3H).

### Example 48

### (R)-3-benzyloxy-1-[2-(cyclooctyl-hydroxy-phenyl-methyl)-oxazol-5-ylmethyl]-1-azonia-bicyclo[2.2.2]octane; formate

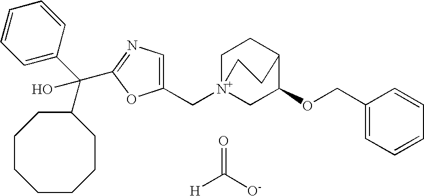

The title compound was prepared from the compound of Step 1 Example 12 and Intermediate 17 by application of General Procedure F. Data for the title compound: LCMS (Method 6, Rt 9.02 min). M⁺=515. ¹H NMR (400 MHz, CDCl₃): δ 8.69 (s, 1H), 7.62-7.57 (m, 2H), 7.38-7.31 (m, 4H), 7.30-7.23 (m, 4H), 7.18 (t, 1H), 4.82 (s, 2H), 4.50-4.41 (m, 2H), 4.09-3.97 (m, 1H), 3.94 (s, 1H), 3.71 (s, 6H), 3.61-3.43 (m, 2H), 3.20-3.06 (m, 2H), 2.63 (d, 1H), 2.30 (s, 1H), 2.19-2.09 (m, 1H), 1.91 (s, 1H), 1.78 (d, 2H), 1.44 (dd, 7H), 1.39-1.31 (m, 2H), 1.27-1.19 (m, 1H).

### Example 49

### (R)-3-(3-fluoro-benzyloxy)-1-[2-(hydroxy-diphenyl-methyl)-oxazol-5-ylmethyl]-1-azonia-bicyclo[2.2.2]octane; formate

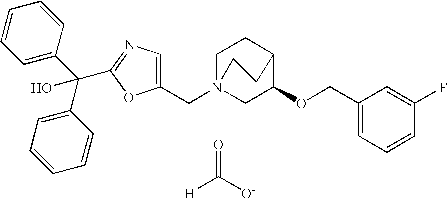

The title compound was prepared from the compound of Step 1 Example 35 and Intermediate 8 by application of General Procedure F. Data for the title compound: LCMS (Method 6, Rt 7.70 min). M⁺=499. ¹H NMR (400 MHz, CDCl₃): δ 8.49 (s, 1H), 7.41-7.29 (m, 6H), 7.28-7.16 (m, 6H), 7.05-6.95 (m, 3H), 5.58 (s, 3H), 4.67-4.50 (m, 2H), 4.38 (s, 2H), 3.91-3.81 (m, 2H), 3.47 (t, 2H), 3.37 (d, 1H), 3.13-2.98 (m, 2H), 2.20 (s, 1H), 2.04 (s, 1H), 1.80 (s, 1H).

### Example 50

### (R)-3-benzyloxy-1-[2-(cyclobutyl-hydroxy-phenyl-methyl)-oxazol-5-ylmethyl]-1-azonia-bicyclo[2.2.2]octane; formate

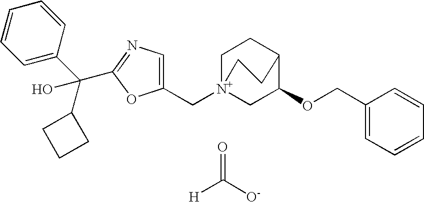

The title compound was prepared from the compound of Step 1 Example 12 and Intermediate 15 by application of General Procedure F. Data for the title compound: LCMS (Method 6, Rt 7.64 min). M⁺=459. ¹H NMR (400 MHz, CDCl₃): δ 8.50 (s, 1H), 7.40-7.27 (m, 6H), 7.29-7.12 (m, 5H), 5.88 (s, 3H), 4.86-4.44 (m, 2H), 4.49-4.36 (m, 2H), 3.89 (d, 2H), 3.52 (d, 2H), 3.44 (s, 1H), 3.28 (p, 1H), 3.08 (d, 2H), 2.30-2.16 (m, 2H), 2.13-1.98 (m, 2H), 1.98-1.88 (m, 2H), 1.84-1.65 (m, 3H).

### Example 51

### (R)-3-allyloxy-1-[2-((R)-cyclohexyl-hydroxy-phenyl-methyl)-oxazol-5-ylmethyl]-1-azonia-bicyclo[2.2.2]octane; formate

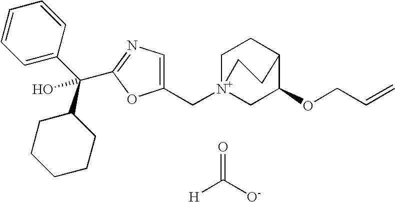

Step 1. (R)-3-Allyloxy-1-aza-bicyclo[2.2.2]octane was prepared from allyl bromide and 3-R-quinuclidinol by the procedure described in Procedure A. Data for (R)-3-allyloxy-1-aza-bicyclo[2.2.2]octane: LCMS (Method 7, Rt 0.38 min). MH⁺=168.

Step 2. The title compound was prepared from the foregoing compound and Intermediate 14 by application of General Procedure F. Data for the title compound: LCMS (Method 6, Rt 7.58 min). M⁺=437. ¹H NMR (400 MHz, CDCl₃): δ 8.62 (s, 1H), 7.60-7.54 (m, 2H), 7.38 (s, 1H), 7.30 (t, 2H), 7.22 (t, 1H), 5.82 (ddt, 1H), 5.38 (s, 4H), 5.27-5.17 (m, 2H), 4.75 (s, 2H), 3.98-3.84 (m, 4H), 3.64-3.38 (m, 3H), 3.15-3.01 (m, 2H), 2.28 (d, 2H), 2.07 (t, 1H), 1.93-1.84 (m, 1H), 1.75 (s, 3H), 1.38-1.24 (m, 3H), 1.24-1.03 (m, 3H).

### Example 52

### (R)-1-[2-((R)-cyclohexyl-hydroxy-phenyl-methyl)-oxazol-5-ylmethyl]-3-(2-fluoro-benzyloxy)-1-azonia-bicyclo[2.2.2]octane; formate

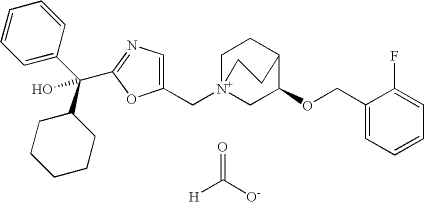

Step 1. (R)-3-(2-Fluoro-benzyloxy)-1-aza-bicyclo[2.2.2]octane was prepared from 2-fluorobenzyl bromide and 3-R-quinuclidinol by the procedure described in Procedure A. Data for (R)-3-(2-fluoro-benzyloxy)-1-aza-bicyclo[2.2.2]octane: LCMS (Method 7, Rt 0.38 min). MH⁺=236.

Step, 2. The title compound was prepared from the foregoing compound and Intermediate 14 by application of General Procedure F. Data for the title compound: LCMS (Method 6, Rt 8.41 min). M⁺=505. ¹H NMR (400 MHz, CDCl₃): δ 8.64 (s, 1H), 7.56-7.51 (m, 2H), 7.35 (s, 1H), 7.35-7.26 (m, 2H), 7.23 (t, 2H), 7.19-7.09 (m, 2H), 7.07-7.00 (m, 1H), 5.26 (s, 3H), 4.81-4.67 (m, 2H), 4.52 (d, 1H), 4.45 (d, 1H), 4.03-3.88 (m, 2H), 3.67-3.39 (m, 3H), 3.11-2.98 (m, 2H), 2.33-2.21 (m, 2H), 2.14-2.01 (m, 1H), 1.95-1.83 (m, 1H), 1.75 (d, 2H), 1.70-1.58 (m, 2H), 1.35-1.14 (m, 3H), 1.18-1.00 (m, 3H).

### Example 53

### (R)-3-(2-cyclohexyl-ethoxy)-1-[2-((R)-cyclohexyl-hydroxy-phenyl-methyl)-oxazol-5-ylmethyl]-1-azonia-bicyclo[2.2.2]octane; formate

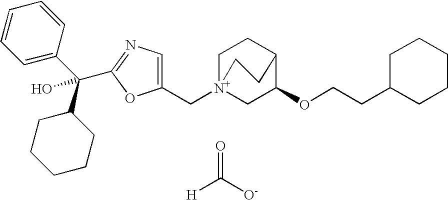

Step 1. (R)-3-(2-Cyclohexyl-ethoxy)-1-aza-bicyclo[2.2.2]octane was prepared from 2-cyclohexylethyl bromide and 3-R-quinuclidinol by the procedure described in Procedure A. Data for (R)-3-(2-Cyclohexyl-ethoxy)-1-aza-bicyclo[2.2.2]octane: LCMS (Method 7, Rt 0.37 min). MH⁺=238.

Step 2. The title compound was prepared from the foregoing compound and Intermediate 14 by application of General Procedure F. Data for the title compound: LCMS (Method 6, Rt 9.65 min). M⁺=507. ¹H NMR (400 MHz, CDCl₃): δ 8.64 (s, 1H), 7.59-7.54 (m, 2H), 7.36 (s, 1H), 7.32-7.25 (m, 2H), 7.25-7.17 (m, 1H), 5.59 (s, 3H), 4.79-4.64 (m, 2H), 3.95-3.86 (m, 1H), 3.75 (s, 1H), 3.60-3.26 (m, 5H), 3.09-2.91 (m, 2H), 2.29 (s, 1H), 2.23 (s, 1H), 2.18-1.84 (m, 1H), 1.91-1.82 (m, 1H), 1.80-1.64 (m, 9H), 1.44-1.32 (m, 2H), 1.33-1.04 (m, 10H), 0.94-0.81 (m, 2H).

### Example 54

### (R)-1-[3-(Cyclohexyl-hydroxy-phenyl-methy)-isoxazol-5-ylmethyl]-3-(4-fluoro-benzyloxy)-1-azonia-bicyclo[2.2.2]octane; bromide

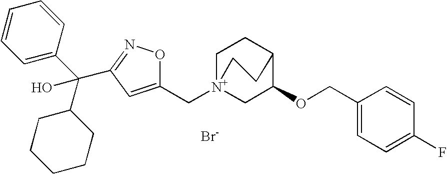

The title compound was prepared from the compound of Step 1 Example 32 and Intermediate 21, Enantiomer 2 by application of General Procedure F. Data for the title compound: LCMS (Method 6, Rt 8.90 min). M⁺=505. ¹H NMR (400 MHz, CDCl₃): δ 7.54 (dd, 2H), 7.30 (t, 4H), 7.27-7.19 (m, 1H), 7.07-7.00 (m, 3H), 5.20 (d, 2H), 4.46 (s, 2H), 4.39 (ddd, 1H), 4.06-4.01 (m, 1H), 3.98 (s, 1H), 3.80 (d, 1H), 3.60 (d, 1H), 3.39 (d, 1H), 3.31 (d, 1H), 2.38 (s, 1H), 2.26 (s, 2H), 2.02-1.97 (m, 1H), 1.94-1.82 (m, 2H), 1.55 (s, 2H), 1.32-1.23 (m, 6H), 1.17-1.05 (m, 3H).

### Example 55

### 1-[2 ((R)—Cyclohexyl-hydroxy-phenyl-methyl)-oxazol-5-ylmethyl]-4-phenoxymethyl-1-azonia-bicyclo[2.2.2]octane; bromide

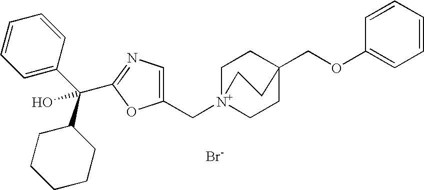

Step 1. A solution of 4-carbethoxyquinuclidine (10.92 g) in THF (155 mL) was treated at −78° C. with borane-THF (1.0 M, 77.5 mL). The resulting mixture was stirred at −78° C. for 4 h, then treated with water (50 mL), warmed to room temperature and stirred for an additional hour. The reaction mixture was diluted with ethyl acetate and the aqueous phase was separated and extracted with two further portions of ethyl acetate. The combined organic layers were washed with brine twice), dried (MgSO₄), filtered and evaporated in vacuo. Purification by silica gel chromatography (eluting with cyclohexane-ethyl acetate [1:0 to 1:1]) gave 1-boranyl-1-aza-bicyclo[2.2.2]octane-4-carboxylic acid ethyl ester (7.12 g, 61%) as an off-white solid. ¹H NMR (400 MHz, CDCl₃) δ 4.16 (2H, q, J=6.9), 3.10-3.05 (6H, m), 1.98-1.93 (6H, m), 1.26 (3H, t, J=6.9).

Step 2. A solution of the foregoing compound (455 mg) was dissolved in ether (15 mL) and cooled to −78° C. The reaction mixture was treated with LiAlH₄ solution (1.0 M in THF) (4.2 mL), stirred for 30 mins, then allowed to warm to room temperature and stirred for an additional 2 h. The reaction mixture was cooled to −40° C. and quenched by the cautious and sequential addition of water (0.24 mL), 4 N NaOH (0.24 mL) and water (0.24 mL). The reaction mixture was warmed to room temperature, stirred for 1 h, and filtered, washing with ether. The filtrate was evaporated and the residue was dissolved in DCM, washed with brine, dried (MgSO₄), filtered and evaporated in vacuo to give (1-boranyl-1-aza-bicyclo[2.2.2]oct-4-yl)-methanol as a white solid (310 mg, 94%). ¹H NMR (300 MHz, CDCl₃) δ 3.41 (2H, d, J=3.2), 3.08-3.03 (6H, m), 1.66-1.59 (6H, m).

Step 3. A suspension of the foregoing compound (200 mg), iodobenzene (0.144 mL), 1,10-phenanthroline (23.2 mg), copper iodide (24.5 mg), Cs₂CO₃ (419 mg) in toluene (0.35 mL) was heated in a sealed vessel for 34 h. The reaction mixture was cooled, filtered through Celite washing with ethyl acetate and DCM. The filtrate was evaporated in vacuo and the resulting residue taken up in acetone and treated with an excess of HCl-MeOH (1.25 M). The reaction mixture was stirred at room temperature for 30 min, evaporated in vacuo and purified by SCX and silica gel chromatography (eluting with 2-10% [2 M NH₃ in MeOH] in DCM) to give 4-phenoxymethyl-1-aza-bicyclo[2.2.2]octane (42 mg, 15%) as a pale-coloured solid. ¹H NMR (300 MHz, CDCl₃) δ 7.27-7.23 (2H, m), 6.96-6.86 (3H, m), 3.59 (2H, s), 2.96-2.90 (6H, m), 1.56-1.49 (6H, m).

Step 4. The title compound was prepared from the foregoing compound and Intermediate 14 by the application of General Procedure F. Data for the title compound: LCMS (Method 6, 8.56 min). M⁺=487.34. ¹H NMR (400 MHz, CDCl₃): δ7.61-7.56 (m, 2H), 7.51 (s, 1H), 7.33-7.26 (m, 3H), 7.25-7.20 (m, 2H), 6.98-6.93 (m, 1H), 6.83-6.80 (m, 2H), 5.25-5.08 (m, 2H), 4.22 (s, 1H), 3.83-3.75 (m, 6H), 3.69 (s, 2H), 2.33 (s, 1H), 1.97 (t, 6H), 1.75 (d, 1H), 1.64 (s, 3H), 1.36 (s, 3H), 1.13 (s, 3H).

### Example 56

### (R)-3-benzyloxy-1-[5-((R)-cyclohexyl-hydroxy-phenyl-methyl)-[1,3,4]oxadiazol-2-ylmethyl]-1-azonia-bicyclo[2.2.2]octane; formate

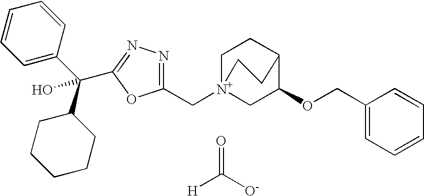

The title compound was prepared from the compound of Step 1 Example 12 and Intermediate 18 by application of General Procedure F. Data for the title compound: LCMS (Method 6, Rt 8.42 min). M⁺=488. ¹H NMR (400 MHz, CDCl₃): δ 8.36 (s, 1H), 7.39-7.23 (m, 5H), 7.22-7.11 (m, 2H), 6.47 (s, 3H), 4.81 (s, 1H), 4.47 (s, 2H), 4.11-4.01 (m, 1H), 3.96 (s, 1H), 3.84-3.58 (m, 4H), 3.47-3.33 (m, 2H), 2.34-2.08 (m, 4H), 1.98-1.91 (m, 1H), 1.87-1.76 (m, 2H), 1.70 (s, 2H), 1.66-1.52 (m, 3H), 1.31-1.14 (m, 3H), 1.16-0.96 (m, 3H).

### Example 57

### (R)-1-[2-(Hydroxy-diphenyl-methyl)-oxazol-5-ylmethyl]-3-(4-trifluoromethyl-phenoxy)-1-azonia-bicyclo[2.2.2]octane; bromide

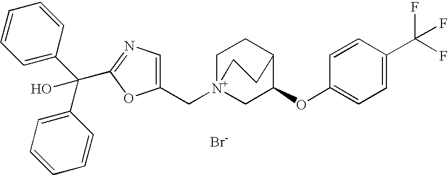

Step 1. (R)-3-(4-Trifluoromethyl-phenoxy)-1-aza-bicyclo[2.2.2]octane was prepared from 1-iodo-4-trifluoromethylbenzene and 3-R-quinuclidinol by the procedure described in General Procedure B. Data for (R)-3-(4-Trifluoromethyl-phenoxy)-1-aza-bicyclo[2.2.2]octane: LCMS (Method 7, Rt 2.39 min). MH⁺=272.

Step 2. The title compound was prepared from the foregoing compound and Intermediate 8 by application of General Procedure F. Data for the title compound: LCMS (Method 6, Rt 8.15 min). M⁺=535. ¹H NMR (400 MHz, CDCl₃): δ 7.55 (d, 2H), 7.51 (s, 1H), 7.42-7.35 (m, 4H), 7.28 (d, 2H), 7.26-7.22 (m, 4H), 6.95 (d, 2H), 6.10 (s, 1H), 4.89 (s, 1H), 4.62 (dd, 3H), 3.92 (t, 1H), 3.81-3.65 (m, 2H), 3.27-3.17 (m, 1H), 3.07 (d, 1H), 2.39 (s, 1H), 2.23-2.12 (m, 1H), 2.09-1.85 (m, 3H).

### Example 58

### (R)-1-[2-(Hydroxy-diphenyl-methyl)-oxazol-5-ylmethyl]-3-p-tolyloxy-1-azonia-bicyclo[2.2.2]octane; bromide

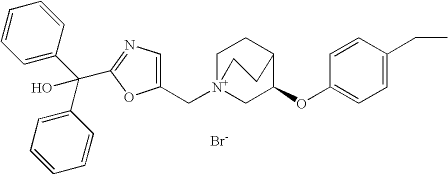

Step 1. (R)-3-(4-methyl-phenoxy)-1-aza-bicyclo[2.2.2]octane was prepared from 1-iodo-4-methylbenzene and 3-R-quinuclidinol by the procedure described in General Procedure B. Data for (R)-3-(4-methyl-phenoxy)-1-aza-bicyclo[2.2.2]octane: LCMS (Method 7, Rt 2.20 min). MH⁺=218.

Step 2. The title compound was prepared from the foregoing compound and Intermediate 8 by application of General Procedure F. Data for the title compound: LCMS (Method 6, Rt 7.95 min). M⁺=481. ¹H NMR (400 MHz, CDCl₃): δ 7.54 (d, 1H), 7.38 (d, 5H), 7.37-7.27 (m, 4H), 7.27 (s, 1H), 7.09 (d, 2H), 6.72 (d, 2H), 5.82 (s, 1H), 4.92 (s, 2H), 4.74 (s, 1H), 4.59-4.51 (m, 1H), 4.16-4.05 (m, 1H), 3.81-3.65 (m, 2H), 3.28-3.14 (m, 2H), 2.59-2.56 (m, 1H), 2.47 (s, 1H), 2.29 (s, 3H), 2.04 (d, 3H).

### Example 59

### (R)-3-(3-Chloro-4-methyl-phenoxy)-1-[2-(hydroxy-diphenyl-methyl)-oxazol-5-ylmethyl]-1-azonia-bicyclo[2.2.2]octane; bromide

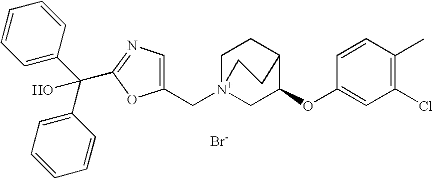

Step 1. (R)-3-(3-chloro-4-methyl-phenoxy)-1-aza-bicyclo[2.2.2]octane was prepared from 1-iodo-3-chloro-4-methylbenzene and 3-R-quinuclidinol by the procedure described in General Procedure B. Data for (R)-3-(3-chloro-4-methyl-phenoxy)-1-aza-bicyclo[2.2.2]octane: LCMS (Method 7, Rt 2.38 min). MH⁺=252.

Step 2. The title compound was prepared from the foregoing compound and Intermediate 8 by application of General Procedure F. Data for the title compound: LCMS (Method 6, Rt 8.33 min). M⁺=517. ¹H NMR (400 MHz, CDCl₃): δ 7.52 (s, 1H), 7.40-7.36 (m, 4H), 7.32-7.26 (m, 4H), 7.26 (s, 2H), 7.12 (d, 1H), 6.86 (d, 1H), 6.66 (dd, 1H), 5.69 (s, 1H), 4.77 (d, 2H), 4.72 (s, 1H), 4.53 (dd, 1H), 4.02 (s, 1H), 3.72 (d, 2H), 3.19 (d, 1H), 3.08 (d, 1H), 2.40 (s, 1H), 2.30 (s, 3H), 2.17 (s, 1H), 2.01-1.94 (m, 2-H), 1.87 (d, 1H).

### Example 60

### (R)-3-(3-Chloro-4-methyl-phenoxy)-1-[2-((R)-cyclohexyl-hydroxy-phenyl-methyl)-oxazol-5-ylmethyl]-1-azonia-bicyclo[2.2.2]octane; bromide

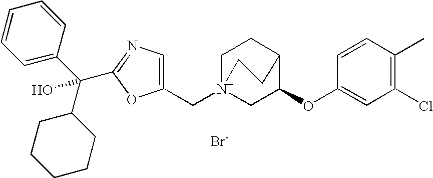

The title compound was prepared from the compound of Step, 1 Example 59 and Intermediate 14 by application of General Procedure F. Data for the title compound: LCMS (Method 6, Rt 9.05 min). M⁺=521. ¹H NMR (400 MHz, CDCl₃): δ 7.59-7.54 (m, 2H), 7.50 (s, 1H), 7.29 (d, 2H), 7.23 (d, 1H), 7.14 (d, 1H), 6.85 (d, 1H), 6.67 (dd, 1H), 5.20-5.01 (m, 2H), 4.79 (s, 1H), 4.73-4.65 (m, 1H), 4.26 (t, 1H), 4.12 (s, 1H), 3.82-3.67 (m, 2H), 3.24 (d, 2H), 2.49 (s, 1H), 2.31 (s, 3. H), 2.26-2.15 (m, 1H), 2.12-2.01 (m, 2H), 1.93-1.82 (m, 1H), 1.75 (d, 1H), 1.66 (s, 3H), 1.37-1.23 (m, 4H), 1.14 (d, 3H).

### Example 61

### 3-benzyl-1-[2-((R)-cyclohexyl-hydroxy-phenyl-methyl)-oxazol-5-ylmethyl]-1-azonia-bicyclo[2.2.2]octane; formate

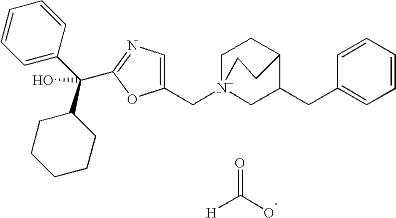

Step 1. (RS)-3-benzyl-1-aza-bicyclo[2.2.2]octane was prepared from benzylmagnesium bromide and 3-quinuclidinone hydrochloride by the procedure described in General Procedure E. Data for (RS)-3-benzyl-1-aza-bicyclo[2.2.2]octane: LCMS (Method 8, Rt 1.90 min). MH⁺ 202.

Step 2. The title compound was prepared from the foregoing compound and Intermediate 14 by application of General Procedure F. Data for the title compound: LCMS (Method 6, Rt 8.38 min). M⁺=471. ¹H NMR (400 MHz, CDCl₃): δ 8.66 (s, 1H), 7.56 (t, 2H), 7.36 (d, 1H), 7.31-7.27 (m, 3H), 7.21 (d, 3H), 7.10 (t, 2H), 4.84-4.67 (m, 2H), 4.65 (s, 3H), 3.62-3.47 (m, 2H), 3.46 (s, 2H), 3.31 (d, 1H), 3.02-2.89 (m, 1H), 2.69 (td, 1H), 2.54 (dt, 1H), 2.29 (s, 2H), 2.05 (s, 1H), 1.89-1.74 (m, 2H), 1.72 (d, 4H), 1.37-1.22 (m, 3H), 1.23-1.03 (m, 3H).

### Example 62

### (R)-3-(3-Chloro-phenoxy)-1-[2-(hydroxy-diphenyl-methyl)-oxazol-5-ylmethyl]-1-azonia-bicyclo[2.2.2]octane; bromide

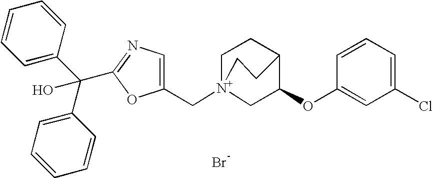

Step 1. (R)-3-(3-chloro-phenoxy)-1-aza-bicyclo[2.2.2]octane was prepared from 1-iodo-3-chlorobenzene and 3-R-quinuclidinol by the procedure described in General Procedure B. Data for (R)-3-(3-chloro-phenoxy)-1-aza-bicyclo[2.2.2]octane: LCMS (Method 7, 2.21 min). MH⁺=238

Step 2. The title compound was prepared from the foregoing compound and Intermediate 8 by application of General Procedure F. Data for the title compound: LCMS (Method 6, Rt 7.99 min). M⁺=501. ¹H NMR (400 MHz, CDCl₃): δ 7.52 (s, 1H), 7.41-7.37 (m, 4H), 7.28 (d, 5H), 7.27-7.18 (m, 3H), 7.00 (dd, 1H), 6.88 (t, 1H), 6.76 (dd, 1H), 5.92 (s, 1H), 4.79-4.65 (m, 2H), 4.59-4.50 (m, 1H), 4.03-3.91 (m, 1H), 3.81-3.65 (m, 2H), 3.21 (q, 1H), 3.09 (d, 1H), 2.39 (s, 1H), 2.17 (dd, 1H), 2.01-1.95 (m, 2H), 1.94-1.82 (m, 1H).

### Example 63

### (R)-3-(4-Chloro-phenoxy)-1-[2-(hydroxy-diphenyl-methyl)-oxazol-5-ylmethyl]-1-azonia-bicyclo[2.2.2]octane; bromide

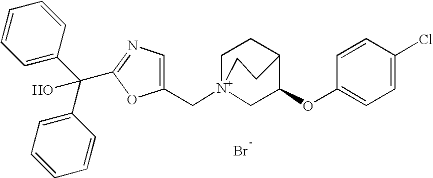

Step 1. (R)-3-(4-chloro-phenoxy)-1-aza-bicyclo[2.2.2]octane was prepared from 1-iodo-4-chlorobenzene and 3-R-quinuclidinol by the procedure described in General Procedure B. Data for (R)-3-(4-chloro-phenoxy)-1-aza-bicyclo[2.2.2]octane: LCMS (Method 7, Rt 2.26 min). MH⁺=238

Step 2. The title compound was prepared from the foregoing compound and Intermediate 8 by application of General Procedure F. Data for the title compound: LCMS (Method 6, Rt 8.02 min). M⁺=501. ¹H NMR (400 MHz, CDCl₃): δ 7.51 (s, 1H), 7.37 (d, 4H), 7.34-7.18 (m, 8H), 6.78 (d, 2H), 5.67 (s, 1H), 4.75 (s, 3H), 4.60 (d, 1H), 4.03 (s, 1H), 3.68 (s, 2H), 3.21 (d, 1H), 3.09 (d, 1H), 2.40 (s, 1H), 2.21 (s, 1H), 2.01 (d, 2H), 1.88 (d, 1H).

### Example 64

### (R)-3-(4-Chloro-phenoxy)-1-[2-((R)-cyclohexyl-hydroxy-phenyl-methyl)-oxazol-5-ylmethyl]-1-azonia-bicyclo[2.2.2]octane; bromide

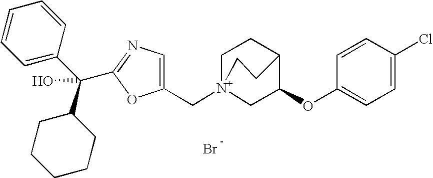

The title compound was prepared from the compound of Step 1 Example 63 and Intermediate 14 by application of General Procedure F. Data for the title compound: LCMS (Method 6, Rt 8.74 min). M⁺=507. ¹H NMR (400 MHz, CDCl₃): δ7.58-7.53 (m, 2H), 7.48 (s, 1H), 7.29-7.24 (m, 3H), 7.25-7.18 (m, 2H), 6.81-6.76 (m, 2H), 5.14-4.99 (m, 2H), 4.82 (s, 1H), 4.77-4.69 (m, 1H), 4.23 (t, 1H), 4.16 (s, 1H), 3.78-3.62 (m, 2H), 3.32-3.19 (m, 2H), 2.47 (s, 1H), 2.34-2.16 (m, 2H), 2.16-1.98 (m, 2H), 1.69 (d, 5H), 1.37-1.23 (m, 4H), 1.15-1.08 (m, 2H).

### Example 65

### (R)-3-(3-Chloro-phenoxy)-1-[2-((R)-cyclohexyl-hydroxy-phenyl-methyl)-oxazol-5-ylmethyl]-1-azonia-bicyclo[2.2.2]octane; bromide

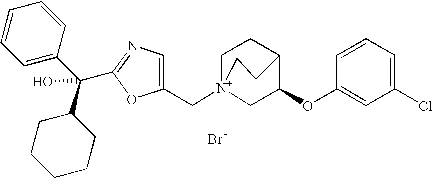

The title compound was prepared from Step 1 Example 62 and Intermediate 14 by application of General Procedure F. Data for the title compound: LCMS (Method 6, Rt 8.74 min). M⁺=507. ¹H NMR (400 MHz, CDCl₃): δ 7.55-7.50 (m, 2H), 7.46 (s, 1H), 7.28-7.21 (m, 2H), 7.22-7.17 (m, 2H), 6.98-6.95 (m, 1H), 6.81 (t, 1H), 6.71 (ddd, 1H), 5.14-4.97 (m, 2H), 4.81 (s, 1H), 4.74-4.66 (m, 1H), 4.22 (t, 1H), 4.10 (s, 1H), 3.78-3.62 (m, 2H), 3.28-3.16 (m, 2H), 2.47 (s, 1H), 2.28 (s, 1H), 2.23-2.11 (m, 1H), 2.10-1.95 (m, 2H), 1.90-1.79 (m, 1H), 1.71 (d, 1H), 1.62 (s, 2H), 1.34-1.19 (m, 4H), 1.16-1.04 (m, 3H).

### Example 66

### (R)-1-[2-((R)—Cyclohexyl-hydroxy-phenyl-methyl)-oxazol-5-ylmethyl]-3-(4-fluoro-phenoxy)-1-azonia-bicyclo[2.2.2]octane; bromide

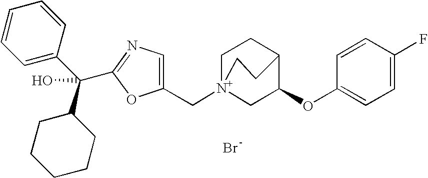

The title compound was prepared from Intermediate 23 and Intermediate 14 by application of General Procedure F. Data for the title compound: LCMS (Method 6, Rt 8.32 min). M⁺=491. ¹H NMR (400 MHz, CDCl₃): δ 7.58-7.53 (m, 2H), 7.48 (s, 1H), 7.28 (d, 2H), 7.21 (d, 1H), 7.01-6.95 (m, 2H), 6.83-6.76 (m, 2H), 5.16-5.00 (m, 2H), 4.78 (s, 1H), 4.74-4.66 (m, 1H), 4.23 (t, 1H), 4.14 (s, 1H), 3.79-3.63 (m, 2H), 3.33-3.20 (m, 2H), 2.47 (s, 1H), 2.27 (dd, 2H), 2.14-1.98 (m, 2H), 1.92-1.81 (m, 1H), 1.74 (d, 1H), 1.66 (s, 2H), 1.38-1.22 (m, 4H), 1.19-1.09 (m, 3H).

### Example 67

### (R)-1-[2-((R)—Cyclohexyl-hydroxy-phenyl-methyl)-oxazol-5-ylmethyl]-3-(3-fluoro-phenoxy)-1-azonia-bicyclo[2.2.2]octane; bromide

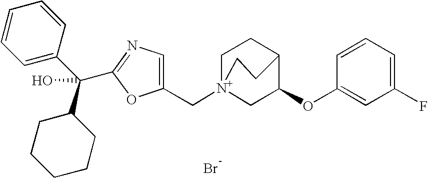

The title compound was prepared from Intermediate 24 and Intermediate 14 by application of General Procedure F. Data for the title compound: LCMS (Method 6, Rt 8.33 min). M⁺=491. ¹H NMR (400 MHz, CDCl₃): δ 7.58-7.54 (m, 2H), 7.49 (s, 1H), 7.28 (dd, 2H), 7.22 (d, 2H), 6.73 (td, 1H), 6.63 (dd, 1H), 6.56 (dt, 1H), 5.17-5.00 (m, 2H), 4.85 (s, 1H), 4.79-4.71 (m, 1H), 4.27 (t, 1H), 4.10 (s, 1H), 3.80-3.64 (m, 2H), 3.29-3.18 (m, 2H), 2.51 (s, 1H), 2.27 (d, 2H), 2.11-2.02 (m, 2H), 1.88 (d, 1H), 1.68 (m, 2), 1.74 (d, 2H), 1.37-1.25 (m, 3H), 1.13 (d, 3H).

### Example 68

### 1-[2-(Hydroxy-diphenyl-methyl)-oxazol-5-ylmethyl]-3-phenylsulfanyl-1-azonia-bicyclo[2.2.2]octane; bromide

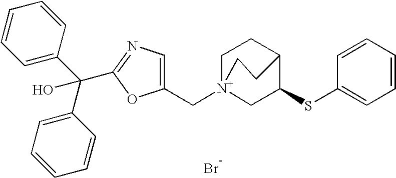

Step 1. (RS)-3-(thiophenoxy)-1-aza-bicyclo[2.2.2]octane was prepared from thiophenol and 3-chloroquinuclidine hydrochloride by the procedure described in Procedure C1. The compound was resolved into its enantiomers by means of chiral HPLC Method 3. Retention time for (S)-enantiomer (Enantiomer 1) is 10.4 min. Retention time for (R)-enantiomer (Enantiomer 2) is 11.5 min. Data for (R)-3-(thiophenoxy)-1-aza-bicyclo[2.2.2]octane: LCMS (Method 7, 2.12 min). MH⁺=220.

Step 2. The title compound was prepared from the foregoing compound (Enantiomer 2) and Intermediate 8 by application of General Procedure F. Data for the title compound: LCMS (Method 6, Rt 7.89 min). M⁺=483. ¹H NMR (400 MHz, CD₃OD): δ 7.46-7.41 (m, 3H), 7.37-7.30 (m, 13H), 4.58 (s, 2H), 3.84 (d, 2H), 3.48-3.45 (m, 5H), 3.24 (m, 1H), 3.12 (t, 1H), 2.21 (d, 1H), 2.17 (m, 1H), 1.98 (m, 2H).

### Example 69

### 1-[2-(R)-cyclohexyl-hydroxy-phenyl-methyl)-oxazol-5-ylmethyl]-3-phenylsulfanyl-1-azonia-bicyclo[2.2.2]octane; formate

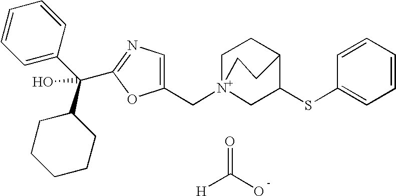

The title compound was prepared from (RS)-3-(thiophenoxy)-1-aza-bicyclo[2.2.2]octane (prepared as described in Example 68, Step 1) and Intermediate 14 by application of General Procedure F. Data for the title compound: LCMS (Method 6, Rt 8.53 min). M⁺=489. ¹H NMR (400 MHz, CD₃OD) δ 8.52 (s, 1H), 7.53-7.48 (m, 2H), 7.47-7.41 (m, 3H), 7.39-7.29 (m, 5H), 7.27-7.22 (m, 1H), 4.55 (s, 2H), 3.84 (s, 2H), 3.53-3.31 (m, 5H), 3.25-3.18 (m, 1H), 2.47-2.34 (m, 2H), 2.23-2.19 (m, 1H), 2.16-2.06 (m, 1H), 2.04-1.89 (m, 2H), 1.76 (d, 1H), 1.73-1.60 (m, 2H), 1.54 (d, 1H), 1.37-1.24 (m, 3H), 1.24-1.06 (m, 3H).

### Example 70

### 1-[3-(Cyclohexyl-hydroxy-phenyl-methyl)-isoxazol-5-ylmethyl]-3-phenylsulfanyl-1-azonia-bicyclo[2.2.2]octane; chloride

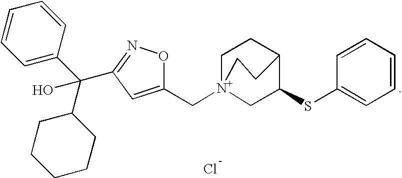

The title compound was prepared from Intermediate 21, Enantiomer 2 and the compound of Step 1 Example 68, Enantiomer 2 by application of General Procedure F. Data for the title compound: LCMS (Method 6, Rt 8.95 min). M⁺=489. ¹H NMR (400 MHz, CDCl₃): δ 7.56-7.52 (m, 2H), 7.41-7.36 (m, 2H), 7.36-7.30 (m, 4H), 7.30 (s, 1-H), 7.20 (t, 1H), 7.05 (s, 1H), 5.22-4.98 (m, 2H), 4.44 (t, 1H), 3.94 (s, 1H), 3.82 (s, 1H), 3.74 (s, 3H), 3.50 (s, 1H), 3.17 (dd, 1H), 2.26-2.15 (m, 1H), 1.93 (s, 8H), 1.75 (s, 1H), 1.27 (d, 4H), 1.16-1.06 (m, 2H).

### Example 71

### 1-[5-((R)—Cyclohexyl-hydroxy-phenyl-methyl)-[1,3,4]oxadiazol-2-ylmethyl]-3-phenylsulfanyl-1-azonia-bicyclo[2.2.2]octane; bromide

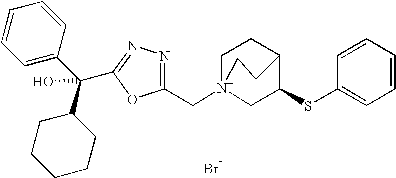

The title compound was prepared from Intermediate 18 and the compound of Step 1 Example 68, Enantiomer 2 by application of General Procedure F. Data for the title compound: LCMS (Method 6, Rt 8.54 min). M⁺=490. ¹H NMR (400 MHz, CDCl₃): δ 7.42 (dd, 2H), 7.38-7.30 (m, 5H), 7.24-7.13 (m, 3H), 4.92 (dd, 2H), 4.42 (t, 1H), 3.84 (d, 4H), 3.63 (s, 1H), 3.34-3.26 (m, 1H), 2.45 (s, 2H), 2.29 (s, 2H), 1.77 (d, 4H), 1.63 (dd, 3H), 1.31-1.22 (m, 3H), 1.16-1.01 (m, 3H).

The following Examples were made from Intermediates 23-31 and Intermediate 8 by Procedure F:

| Exam- | | | Rt | |
|---|---|---|---|---|
| ple | | | Method | |
| Number | Structure | Name | M⁺ | NMR |
| Exam- ple 72 | 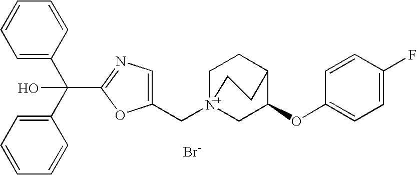 | (R)-3-(4-Fluoro- phenoxy)-1-[2- (hydroxy-diphenyl- methyl)-oxazol-5- ylmethyl]-1-azonia- bicyclo[2.2.2]octane; bromide | 7.62 6 485 | ¹H NMR (400 MHz, CDCl₃): δ 7.54-7.52 (m, 1 H), 7.38- 7.35 (m, 3 H), 7.30 (d, 7 H), 7.02-6.96 (m, 2 H), 6.80 (dd, 2 H), 5.40 (s, 1 H), 4.83 (s, 2 H), 4.73 (s, 1 H), 4.65- 4.58 (m, 1 H), 4.12 (s, 1 H), 3.67 (d, 2 H), 3.22 (s, 1 H), 3.12 (d, 1 H), 2.44 (s, 1 H), 2.24 (t, 1H), 2.06-1.99 (m, 2 H), 1.94 (m, 1H). |
| Exam- ple 73 | 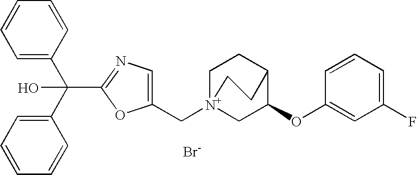 | (R)-3-(3-Fluoro- phenoxy)-1-[2- (hydroxy-diphenyl- methyl)-oxazol-5- ylmethyl]-1-azonia- bicyclo[2.2.2]octane; bromide | 7.67 6 485 | ¹H NMR (400 MHz, CDCl₃): δ 7.52 (d, 1 H), 7.38 (d, 4 H), 7.38-7.14 (m, 7 H), 6.73 (t, 1 H), 6.64 (d, 1 H), 6.58 (d, 1 H), 5.62-5.83 (m, 1 H), 4.77 (s, 3 H), 4.58 (s, 1 H), 4.03 (s, 1 H), 3.73 (d, 2 H), 3.21 (d, 1 H), 3.09 (d, 1 H), 2.42 (s, 1 H), 2.19 (d, 1 H), 2.01 (s, 2 H), 1.89 (d, 1 H). |
| Exam- ple 74 | 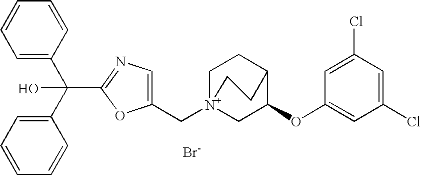 | (R)-3-(3,5-dichloro- phenoxy)-1-[2- (hydroxy-diphenyl- methyl)-oxazol-5- ylmethyl]-1-azonia- bicyclo[2.2.2]octane; bromide | 8.50 6 537 | ¹H NMR (400 MHz, CDCl₃): δ 7.48 (s, 1 H), 7.42-7.33 (m, 4 H), 7.33-7.21 (m, 3 H), 7.20 (d, 3 H), 6.99 (t, 1 H), 6.78 (d, 2 H), 6.02 (s, 1 H), 4.73 (d, 2 H), 4.61-4.45 (m, 2 H), 3.89 (s, 1 H), 3.78-3.60 (m, 2 H), 3.21 (d, 1 H), 3.09 (d, 1 H), 2.31 (s, 1 H), 2.05 (s, 1 H), 1.91 (s, 2 H), 1.84 (d, 1 H). |
| Exam- ple 75 | 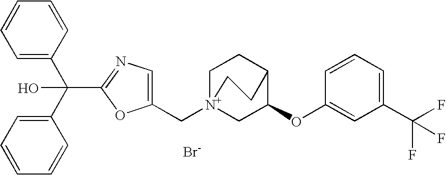 | (R)-1-[2-[Hydroxy- diphenyl-methyl)- oxazol-5-ylmethyl]- 3-(3-trifluoromethyl- phenoxy)-1-azonia- bicyclo[2.2.2]octane; bromide | 8.34 6 535 | ¹H NMR (400 MHz, CDCl₃): δ 7.54-7.51 (m, 1 H), 7.42 (d, 2 H), 7.36-7.19 (m, 5 H), 7.26 (s, 5 H), 7.11-7.03 (m, 2 H), 5.68 (s, 1 H), 4.87 (s, 1 H), 4.77 (s, 2 H), 4.68 (s, 1 H), 4.08 (s, 1 H), 3.73 (s, 2 H), 3.23 (s, 1 H), 3.11 (d, 1 H), 2.44 (s, 1 H), 2.21 (s, 1 H), 2.04 (dd, 2 H), 1.91 (d, 1 H). |
| Exam- ple 76 | 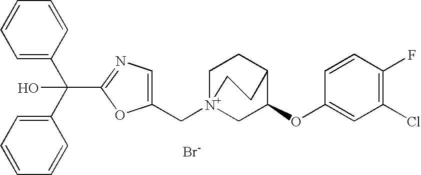 | (R)-3-(3-chloro-4- fluoro-phenoxy)-1-[2- (hydroxy-diphenyl- methyl)-oxazol-5- ylmethyl]-1-azonia- bicyclo[2.2.2]octane; bromide | 8.05 6 519 | ¹H NMR (400 MHz, CDCl₃): δ 7.48 (d, 1 H), 7.38-7.34 (m, 4 H), 7.28-7.23 (m, 3 H), 7.23- 7.19 (m, 3 H), 7.02 (t, 1 H), 6.91 (dd, 1 H), 6.73 (dt, 1 H), 6.06 (s, 1 H), 4.70-4.53 (m, 3 H), 4.53-4.44 (m, 1 H), 3.85 (s, 1 H), 3.75-3.58 (m, 2 H), 3.25-3.15 (m, 1 H), 3.06 (d, 1 H), 2.30 (s, 1 H), 2.16- 2.05 (m, 1 H), 1.95-1.78 (m, 3H). |
| Exam- ple 77 | 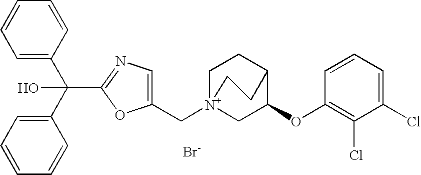 | (R)-3-(2,3-dichloro- phenoxy)-1-[2- (hydroxy-diphenyl- methyl)-oxazol-5- ylmethyl]-1-azonia- bicyclo[2.2.2]octane; bromide | 8.31 6 537 | ¹H NMR (400 MHz, DMSO- d₆): δ 7.53 (s, 1 H), 7.37-7.28 (m, 10 H), 7.29-7.24 (m, 2 H), 7.17-7.11 (m, 2 H), 5.10- 5.04 (m, 1 H), 4.78-4.67 (m, 2 H), 3.87 (dd, 1 H), 3.58- 3.30 (m, 6 H), 2.15 (s, 1 H), 2.08-1.99 (m, 1 H), 1.89 (d, 2 H). |
| Exam- ple 78 | 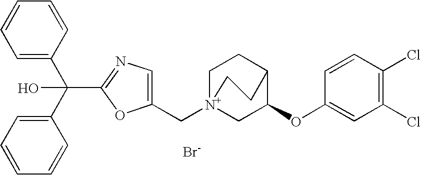 | (R)-3-(3,4-dichloro- phenoxy)-1-[2- (hydroxy-diphenyl- methyl)-oxazol-5- ylmethyl]-1-azonia- bicyclo[2.2.2]octane; bromide | 8.49 6 537 | ¹H NMR (400 MHz, DMSO- d₆): δ 7.59 (d, 1 H), 7.54 (s, 1 H), 7.39-7.30 (m, 8 H), 7.30- 7.23 (m, 3 H), 7.12 (s, 1 H), 7.01 (dd, 1 H), 4.98 (s, 1 H), 4.76-4.65 (m, 2 H), 3.88 (dd, 1 H), 3.54-3.34 (m, 5 H), 2.44 (s, 1 H), 2.14-1.95 (m, 2 H), 1.95-1.78 (m, 2 H). |
| Exam- ple 79 | 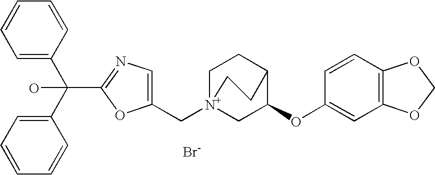 | (R)-3-(Benzo[1,3] dioxal-5-yloxy)-1-[2- (hydroxy-diphenyl- methyl)-oxazol-5- ylmethyl]-1-azonia- bicyclo[2.2.2]octane; bromide | 7.51 6 511 | ¹H NMR (400 MHz, DMSO- d₆): δ 7.54 (s, 1 H), 7.39-7.33 (m, 5 H), 7.33-7.24 (m, 5 H), 7.13 (s, 1 H), 6.84 (d, 1 H), 6.71 (d, 1 H), 6.41 (dd, 1 H), 5.99 (s, 2 H), 4.77 (s, 2 H), 4.71 (d, 2 H), 3.87-3.76 (m, 1 H), 3.50-3.33 (m, 4 H), 2.38 (s, 1 H), 2.13 (s, 1 H), 2.03- 1.94 (m, 1 H), 1.93-1.78 (m, 2 H). |
| Exam- ple 80 | 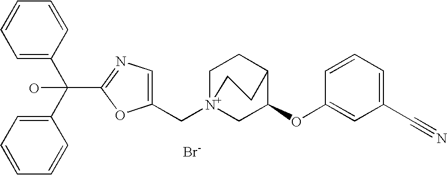 | (R)-3-(3-cyano- phenoxy)-1-[2- (hydroxy-diphenyl- methyl)-oxazol-5- ylmethyl]-1-azonia- bicyclo[2.2.2]octane; bromide | 7.41 6 492 | ¹H NMR (400 MHz, DMSO- d₆): δ 7.56-7.45 (m, 4 H), 7.38-7.29 (m, 8 H), 7.30-7.23 (m, 3 H), 7.11 (s, 1 H), 5.01 (s, 1 H), 4.76-4.66 (m, 2 H), 3.90 (dd, 1 H), 3.51-3.29 (m, 5 H), 2.45 (s, 1 H), 2.10 (s, 1 H), 2.06-1.97 (m, 1 H), 1.95- 1.80 (m, 2 H). |

### Example 81

### 1-[2-((R)—Cyclohexyl-hydroxy-phenyl-methyl)-oxazol-5-ylmethyl]-3-(3-fluoro-phenoxymethyl)-1-azonia-bicyclo[2.2.2]octane; bromide

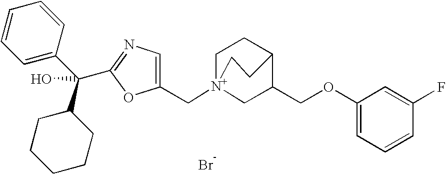

Step 1. 3-(3-Fluoro-phenoxymethyl)-1-aza-bicyclo[2.2.2]octane was prepared from 1-iodo-3-fluoro-benzene and (1-aza-bicyclo[2.2.2]oct-3-yl)-methanol by the procedure described for Step 1 Example 24. LCMS (Method 7, Rt 0.38 min). MH⁺=236. A portion of this compound (0.2 g) was separated into its enantiomers by use of HPLC Method 3, to give Enantiomer 1 (Retention time=9.16 min) (81.7 mg) and Enantiomer 2 (Retention time=10.67 min) (104 mg), both as colourless oils.

Step 2. The title compound was prepared from the foregoing compound (Enantiomer 2) and Intermediate 14 by application of General Procedure F. Data for the title compound: LCMS (Method 6, Rt 8.63 min). M⁺=505.09. ¹H NMR (400 MHz, CD₃OD) δ 7.56-7.50 (m, 2H), 7.48 (s, 1H), 7.34-7.18 (m, 4H), 6.76-6.67 (m, 3H), 4.61 (s, 2H), 4.09-4.01 (m, 2H), 3.69 (t, 1H), 3.52-3.39 (m, 5H), 3.19 (ddd, 1H), 2.72-2.62 (m, 1H), 2.45-2.37 (m, 1H), 2.26 (d, 1H), 2.17 (d, 1H), 2.07 (dd, 2H), 1.93 (dd, 1H), 1.78-1.59 (m, 3H), 1.55 (d, 1H), 1.32 (d, 3H), 1.26-1.01 (m, 3H).

### Example 82

### 1-[2-((R)—Cyclohexyl-hydroxy-phenyl-methyl)-oxazol-5-ylmethyl]-3-(4-fluoro-phenoxymethyl)-1-azonia-bicyclo[2.2.2]octane; bromide

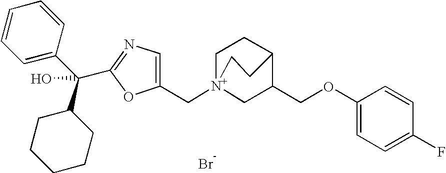

Step 1. 3-(4-Fluoro-phenoxymethyl)-1-aza-bicyclo[2.2.2]octane was prepared from 1-iodo-4-fluoro-benzene and (1-aza-bicyclo[2.2.2]oct-3-yl)-methanol by the procedure described for Step 1 Example 24. LCMS (Method 7, Rt 0.38 min). MH⁺=236. A portion of this compound (0.2 g) was separated into its enantiomers by use of HPLC Method 3, to give Enantiomer 1 (Retention time=9.71 min) (82.4 mg) and Enantiomer 2 (Retention time=11.33 min) (102 mg), both as colourless oils.

Step 2. The title compound was prepared from the foregoing compound (Enantiomer 2) and Intermediate 14 by application of General Procedure F. Data for the title compound: LCMS (Method 6, Rt 8.51 min). M⁺=505. ¹H NMR (400 MHz, CD₃OD) δ 7.56-7.51 (m, 2H), 7.47 (s, 1H), 7.35-7.27 (m, 2H), 7.25-7.19 (m, 1H), 7.04-6.97 (m, 2H), 6.94-6.87 (m, 2H), 4.60 (s, 2H), 4.05-3.96 (m, 2H), 3.68 (ddd, 1H), 3.51-3.35 (m, 5H), 3.19 (ddd, 1H), 2.70-2.59 (m, 1H), 2.44-2.35 (m, 1H), 2.26 (d, 1H), 2.18 (d, 1H), 2.06 (dd, 2H), 1.98-1.88 (m, 1H), 1.80-1.57 (m, 3H), 1.54 (s, 1H), 1.32 (d, 3H), 1.25-1.00 (m, 3H).

### Example 83

### 3-(3-Fluoro-phenoxymethyl)-1-[2-(hydroxy-diphenyl-methyl)-oxazol-5-ylmethyl]-1-azonia-bicyclo[2.2.2]octane; bromide

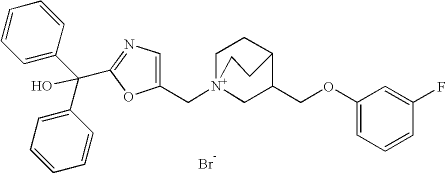

The title compound was prepared from the compound of Step 1 Example 81 (Enantiomer 1) and Intermediate 8 by application of General Procedure F. Data for the title compound: LCMS (Method 6, Rt 7.87 min). M⁺=498. ¹H NMR (400 MHz, CD₃OD) δ 7.52 (s, 1H), 7.38-7.33 (m, 5H), 7.33-7.24 (m, 6H), 6.77-6.68 (m, 3H), 4.64 (s, 2H), 4.10-4.00 (m, 2H), 3.69 (ddd, 1H), 3.52-3.38 (m, 5H), 3.21 (ddd, 1H), 2.73-2.63 (m, 1H), 2.27 (d, 1H), 2.21-2.02 (m, 3H), 1.93 (dd, 1H).

### Example 84

### 3-(3-Fluoro-phenoxymethyl)-1-[2-(hydroxy-diphenyl-methyl)-oxazol-5-ylmethyl]-1-azonia-bicyclo[2.2.2]octane; bromide

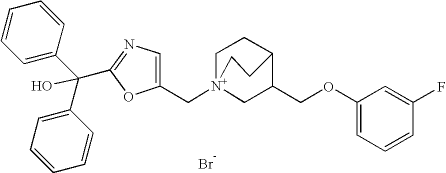

The title compound was prepared from the compound of Step 1 Example 81 (Enantiomer 2) and Intermediate 8 by application of General Procedure F. Data for the title compound: LCMS (Method 6, Rt 7.94 min). M⁺=499.02. ¹H NMR (400 MHz, CD₃OD): δ 7.51 (s, 1H), 7.37-7.32 (m, 5H), 7.32-7.24 (m, 6H), 6.76-6.67 (m, 3H), 4.63 (s, 2H), 4.09-3.99 (m, 2H), 3.68 (ddd, 1H), 3.50-3.36 (m, 5H), 3.20 (ddd, 1H), 2.72-2.62 (m, 1H), 2.26 (d, 1H), 2.22-2.02 (m, 3H); 1.93 (dd, 1H).

### Example 85

### 1-[2-(R)-cyclohexyl-hydroxy-phenyl-methyl)-oxazol-5-ylmethyl]-3-(3-fluoro-phenoxymethyl)-1-azonia-bicyclo[2.2.2]octane; formate

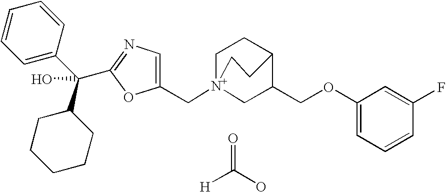

The title compound was prepared from the compound of Step 1 Example 81 (Enantiomer 1) and Intermediate 14 by application of General Procedure F. Data for the title compound: LCMS (Method 6, Rt 8.67 min). M⁺=505. ¹H NMR (400 MHz, CD₃OD) δ 8.50 (s, 1H), 7.53-7.49 (m, 2H), 7.46 (s, 1H), 7.32-7.19 (m, 4H), 6.75-6.67 (m, 3H), 4.57 (s, 2H), 4.09-3.99 (m, 2H), 3.67 (ddd, 1H), 3.51-3.38 (m, 4H), 3.20-3.12 (m, 1H), 2.71-2.60 (m, 1H), 2.44-2.36 (m, 1H), 2.26 (d, 1H), 2.22-2.12 (m, 1H), 2.12-2.01 (m, 2H), 1.97-1.87 (m, 1H), 1.78-1.51 (m, 4H), 1.37-1.01 (m, 7H).

### Example 86

### 3-(4-Fluoro-phenoxymethyl)-1-[2-(hydroxy-diphenyl-methyl)-oxazol-5-ylmethyl]-1-azonia-bicyclo[2.2.2]octane; bromide

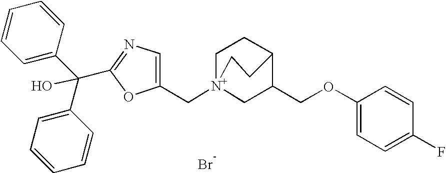

The title compound was prepared from the compound of Step 1 Example 82 (Enantiomer 2) and Intermediate 8 by application of General Procedure F. Data for the title compound: LCMS (Method 6, Rt 7.88 min). M⁺=499 ¹H NMR (400 MHz, CD₃OD): δ 7.52 (s, 1H), 7.38-7.25 (m, 10H), 7.05-6.98 (m, 2H), 6.94-6.87 (m, 2H), 4.63 (s, 2H), 4.05-3.96 (m, 2H), 3.68 (ddd, 1H), 3.52-3.38 (m, 5H), 3.21 (ddd, 1H), 2.71-2.61 (m, 1H), 2.27 (d, 1H), 2.23-2.03 (m, 3H), 1.98-1.88 (m, 1H).

### Example 87

### 1-[2-((R)—Cyclohexyl-hydroxy-phenyl-methyl)-oxazol-5-ylmethyl]-3-(4-fluoro-phenoxymethyl)-1-azonia-bicyclo[2.2.2]octane; bromide

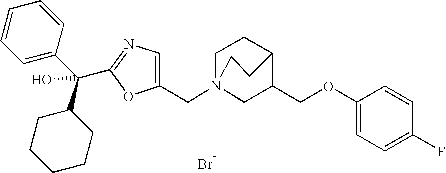

The title compound was prepared from the compound of Step 1 Example 82 (Enantiomer 1) and Intermediate 14 by application of General Procedure F. Data for the title compound: LCMS (Method 6, Rt 8.52 min). M⁺=505. ¹H NMR (400 MHz, CD₃OD): δ 7.54-7.49 (m, 2H), 7.46 (s, 1H), 7.33-7.26 (m, 2H), 7.24-7.19 (m, 1H), 7.03-6.96 (m, 2H), 6.93-6.86 (m, 2H), 4.59 (s, 2H), 4.04-3.96 (m, 2H), 3.72-3.64 (m, 1H), 3.52-3.40 (m, 5H), 3.19 (ddd, 1H), 2.70-2.59 (m, 1H), 2.40 (t, 1H), 2.26-2.11 (m, 2H), 2.10-2.01 (m, 2H), 1.97-1.87 (m, 1H), 1.77-1.50 (m, 4H), 1.33-1.01 (m, 6H).

### Example 88

### 1-[2-((R)—Cyclohexyl-hydroxy-phenyl-methyl)-oxazol-5-ylmethyl]-3-phenyl-1-azonia-bicyclo[2.2.2]octane; bromide

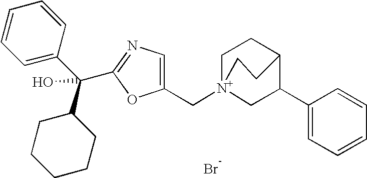

Step 1. 3-Phenylquinuclidine was prepared from 3-quinuclidinone hydrochloride and phenylmagnesium bromide by procedures analogous to General Procedure E. Data for 3-phenylquinuclidine: LCMS (Method 7, Rt 1.93 min). MH⁺=188.

Step 2. The title % compound was prepared from the foregoing compound and Intermediate 14 by application of General Procedure F. Data for the title compound: LCMS (Method 6, Rt 8.23 min). M⁺=457. ¹H NMR (400 MHz, CDCl₃): δ7.62-7.58 (m, 2H), 7.49 (d, 1H), 7.45-7.09 (m, 8H), 5.32-5.12 (m, 2H), 4.17 (t, 1H), 3.85 (d, 2H), 3.74 (dd, 4H), 3.52-3.42 (m, 1H), 2.32 (s, 2H), 2.23-2.11 (m, 3H), 1.41-1.33 (m, 3H), 1.28 (d, 4H), 1.14 (s, 4H).

### Example 89

### 3-Allyloxymethyl-1-[2-((R)-cyclohexyl-hydroxy-phenyl-methyl)-oxazol-5-ylmethyl]-1-azonia-bicyclo[2.2.2]octane; formate

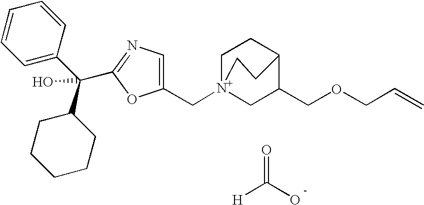

Step 1. 3-(Allyloxymethyl)-1-aza-bicyclo[2.2.2]octane was prepared from allyl bromide and (1-aza-bicyclo[2.2.2]oct-3-yl)-methanol by the procedure described in General Procedure A. LCMS (Method 7, Rt 0.38 min). MH⁺=182. A portion of this compound (0.15 g) was separated into its enantiomers by use of chiral HPLC Method 3, to give Enantiomer 1 (Retention time=8.54 min) (50.8 mg) and Enantiomer 2 (Retention time=11.11 min) (41 mg), both as colourless oils.

Step 2. The title compound was prepared from the foregoing compound (Enantiomer 2) and Intermediate 14 by application of General Procedure F. Data for the title compound: LCMS (Method 6, Rt 7.75 min). M⁺=451. ¹H NMR (400 MHz, CDCl₃): δ 8.67 (s, 1H), 7.59-7.52 (m, 2H), 7.37 (s, 1H), 7.30-7.23 (m, 2H), 7.19 (t, 1H), 5.80 (ddt, 1H), 5.24-5.13 (m, 2H), 4.86-4.59 (m, 3H), 3.94-3.84 (m, 2H), 3.61-3.46 (m, 2H), 3.39 (d, 2H), 3.30 (d, 2H); 2.94 (dd, 1H), 2.24 (s, 2H), 2.02-1.86 (m, 2H), 1.80 (t, 3H), 1.67 (s, 4H), 1.23 (s, 4H), 1.17-0.98 (m, 3H).

### Example 90

### 3-Allyloxymethyl-1-[2-((R)-cyclohexyl-hydroxy-phenyl-methyl)-oxazol-5-ylmethyl]-1-azonia-bicyclo[2.2.2]octane; formate

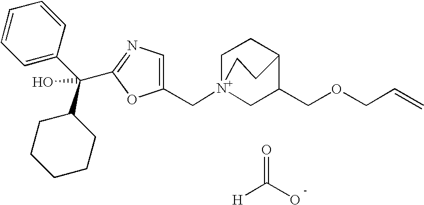

The title compound was prepared from the compound of Step 1 Example 89, Enantiomer 1 and Intermediate 14 by application of General Procedure F. Data for the title compound: LCMS (Method 6, Rt 7.84 min). M⁺=451. ¹H NMR (400 MHz, CDCl₃): δ 8.67 (s, 1H), 7.59-7.52 (m, 2H), 7.39 (s, 1H), 7.32-7.26 (m, 2H), 7.21 (t, 1H), 5.82 (ddt, 1H), 5.26-5.16 (m, 2H), 4.85-4.66 (m, 3H), 3.97-3.87 (m, 2H), 3.63-3.53 (m, 2H), 3.50-3.24 (m, 4H), 2.94 (dd, 1H), 2.29 (s, 3H), 2.04 (s, 1H), 1.99 (s, 1H), 1.92-1.77 (m, 3H), 1.73 (d, 4H), 1.33-1.23 (m, 3H), 1.19-1.06 (m, 3H).

### Example 91

### (S)-1-[3-(Cyclohexyl-hydroxy-phenyl-methyl)-isoxazol-5-ylmethyl]-3-phenoxymethyl-1-azonia-bicyclo[2.2.2]octane; chloride

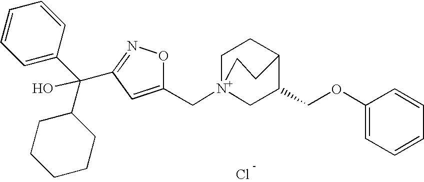

The title compound was made from Intermediate 21, enantiomer 2 and the compound of Method 2 (Step 1), Example 24 by application of General Procedure F. Data for the title compound: LCMS (Method 6, Rt 8.85 min). M⁺=487. ¹H NMR (400 MHz, DMSO-d₆): δ 7.48 (dd, 2H), 7.33-7.27 (m, 4H), 7.24-7.18 (m, 1H), 6.98-6.91 (m, 3H), 6.79 (d, 1 H), 5.91 (s, 1H), 4.70 (s, 2H), 4.09-4.00 (m, 2H), 3.71-3.63 (m, 1H), 3.54-3.36 (m, 4H), 2.60 (t, 1H), 2.24-2.11 (m, 2H), 2.03-1.86 (m, 3H), 1.83 (t, 1H), 1.67 (d, 1H), 1.59 (s, 4H), 1.32-1.14 (m, 3H), 1.15-0.99 (m, 3H).

### Example 92

### (S)-1-[2-(Hydroxy-diphenyl-methyl)-oxazol-5-ylmethyl]-3-phenoxymethyl-1-azonia-bicyclo[2.2.2]octane; bromide

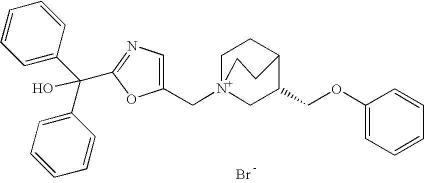

The title compound was made from the compound of Step 1 Example 24, Enantiomer 1 and Intermediate 8 by application of General Procedure F. Data for the title compound: LCMS (Method 6, Rt 7.78 min). M⁺=481. ¹H NMR (400 MHz, DMSO-d₆): δ 7.53 (s, 1H), 7.39-7.22 (m, 11H), 7.11 (s, 1H), 6.96 (dd, 3H), 4.63 (s, 2H), 4.03 (d; 2H), 3.67-3.55 (m, 1H), 3.50-3.32 (m, 5H), 3.22-3.13 (m, 1H), 2.62-2.54 (m, 1H), 2.16 (s, 1H), 2.07 (m, 1H), 1.94 (d, 2H), 1.82 (t, 1H).

### Example 93

### (S)-1-[3-(Hydroxy-diphenyl-methyl)-[1,2,4]oxadiazol-5-ylmethyl]-3-phenoxymethyl-1-azonia-bicyclo[2.2.2]octane; chloride

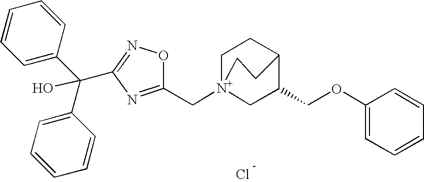

The title compound was made from the compound of Step 1 Example 24, Enantiomer 1 and Intermediate 22 by application of General Procedure F. Data for the title compound: LCMS (Method 6, Rt 7.97 min). M⁺=482. ¹H NMR (400 MHz, DMSO-d₆): δ 7.40-7.36 (m, 4H), 7.33-7.24 (m, 8H), 7.15 (s, 1H), 6.99-6.93 (m, 3H), 4.97 (s, 2H), 4.12-4.02 (m, 2H), 3.83 (t, 1H), 3.66-3.59 (m, 4H), 3.42 (dd, 1H), 2.69-2.57 (m, 1H), 2.18 (d, 1H), 2.09 (s, 1H), 1.98 (s, 2H), 1.95-1.77 (m, 1H).

### Example 94

### (R)-3-(3-Fluoro-phenoxy)-1-[3-(hydroxy-diphenyl-methyl)-[1,2,4]oxadiazol-5-ylmethyl]-1-azonia-bicyclo[2.2.2]octane; chloride

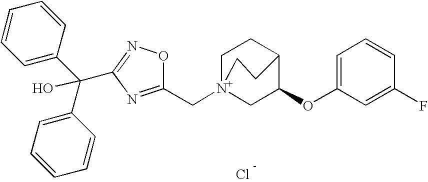

The title compound was made from Intermediate 24 and Intermediate 22 by application of General Procedure F. Data for the title compound: LCMS (Method 6, Rt 7.91 min). M⁺=486. ¹H NMR (400 MHz, DMSO-d₆): δ 7.40-7.35 (m, 5H), 7.34-7.25 (m, 7H), 7.15 (s, 1H), 6.92-6.80 (m, 3H), 5.11-4.99 (m, 2H), 4.98 (s, 1H), 4.16-4.07 (m, 1H), 3.71-3.50 (m, 4H), 2.47 (s, 1H), 2.16 (s, 1H), 2.12-2.02 (m, 1H), 2.00-1.83 (m, 2H).

### Example 95

### (R)-3-(4-Fluoro-phenoxy)-1-[3-(hydroxy-diphenyl-methyl)-[1,2,4]oxadiazol-5-ylmethyl]-1-azonia-bicyclo[2.2.2]octane; chloride

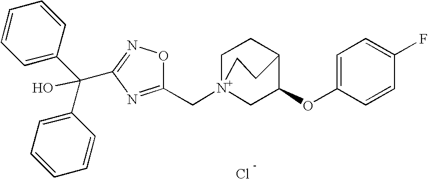

The title compound was made from Intermediate 23 and Intermediate 22 by application of General Procedure F. Data for the title compound: LCMS (Method 6, Rt 7.94 min). M⁺=486. ¹H NMR (400 MHz, DMSO-d₆): δ 7.38 (dd, 4H), 7.35-7.26 (m, 6H), 7.22-7.13 (m, 3H), 7.03-6.96 (m, 2H), 5.05 (s, 2H), 4.89 (s, 1H), 4.12-4.03 (m, 1H), 3.70-3.49 (m, 5H), 2.43 (s, 1H), 2.17 (s, 1H), 2.12-2.00 (m, 1H), 1.98-1.82 (m, 2H).

### Example 96

### (R)-1-[2-((R)—Cyclohexyl-hydroxy-phenyl-methyl)-oxazol-5-ylmethyl]-3-(tetrahydro-pyran-2-yloxy)-1-azonia-bicyclo[2.2.2]octane; bromide

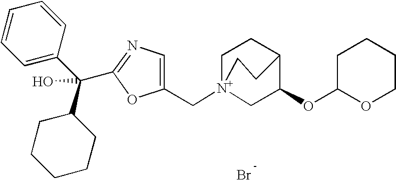

Step 1. A solution of R-quinuclidinol (1.5 g) in DCM (30 mL) was treated with methanesulfonic acid (1.19 g) and 3,4-dihydro-2H-pyran (1.98 g) and stirred at room temperature for 1 h. The reaction mixture was poured into saturated aqueous potassium carbonate solution and extracted with ethyl acetate. The organic extract was washed with brine, dried (MgSO₄), filtered and evaporated in vacuo. Purification by silica gel chromatography (eluting with 0-10% [2M NH₃-MeOH]-DCM) gave 3-(tetrahydro-pyran-2-yloxy)-1-aza-bicyclo[2.2.2]octane (2.1 g, 84%) as a pale straw-coloured oil.

Step 2. The title compound (35 mg, 33%) was prepared from the foregoing compound and Intermediate 14 by General Procedure F. Data for the title compound: LCMS (Method 7, Rt 7.95 min). M⁺=481. ¹H NMR (400 MHz, DMSO-d₆): δ 7.50-7.45 (m, 3H), 7.35-7.28 (m, 2H), 7.24 (t, 1H), 6.08 (s, 1H), 4.68-4.60 (m, 3H), 4.16-4.10 (m, 1H), 3.74-3.66 (m, 1H), 3.49-3.39 (m, 2H), 2.30-2.19 (m, 2H), 2.12-1.86 (m, 2H), 1.64 (s, 7H), 1.46 (s, 5H), 1.39 (s, 5H), 1.30-1.13 (m, 3H), 1.16-0.92 (m, 3H).

### Example 97

### (R)-3-(4-Fluoro-phenylsulfanyl)-1-[2-(hydroxy-diphenyl-methyl)-oxazol-5-ylmethyl]-1-azonia-bicyclo[2.2.2]octane; bromide

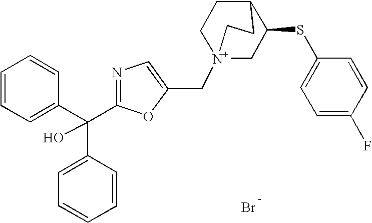

Step 1. (R)-3-(4-Fluoro-phenylsulfanyl)-1-aza-bicyclo[2.2.2]octane was prepared from methanesulfonic acid (S)-(1-aza-bicyclo[2.2.2]oct-3-yl) ester and 4-fluoro benzenethiol by analogy to the procedure described in General Procedure C2. ¹H NMR (300 MHz, CH₃OH-d₄): δ 7.52-7.44 (m, 2H), 7.12-7.03 (m, 2H), 3.58 (ddt, 1H), 3.50-3.40 (m, 1H), 3.13-2.89 (m, 4H), 2.83-2.73 (m, 1H), 2.30-2.17 (m, 1H), 1.96-1.84 (m, 2H), 1.80-1.70 (m, 1H), 1.69-1.54 (m, 1H).

Step 2. The title compound was prepared from the foregoing compound and Intermediate 8 by application of General Procedure F. Data for the title compound: LCMS (Method 6, Rt 8.05 min). M+=501. ¹H NMR (400 MHz, CH₃OH-d₄): δ 7.53-7.47 (m, 3H), 7.37-7.28 (m, 10H), 7.14-7.07 (m, 2H), 4.59 (s, 2H), 3.85-3.72 (m, 2H), 3.54-3.31 (m, 5H), 3.24 (ddd, 1H), 2.48-2.38 (m, 1H), 2.21-2.17 (m, 1H), 2.15-2.06 (m, 1H), 2.01-1.91 (m, 2H).

### Example 98

### (R)-3-(3-Fluoro-phenylsulfanyl)-1-[2-(hydroxy-diphenyl-methyl)-oxazol-5-ylmethyl]-1-azonia-bicyclo[2.2.2]octane; bromide

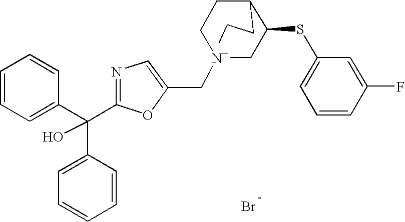

Step 1. (R)-3-(3-Fluoro-phenylsulfanyl)-1-aza-bicyclo[2.2.2]octane was prepared from methanesulfonic acid (S)-(1-aza-bicyclo[2.2.2]oct-3-yl) ester and 3-fluoro benzenethiol by analogy to the procedure described in General Procedure C2. ¹H NMR (300 MHz, CH₃OH-d₄): δ 7.33 (td, 1H), 7.22-7.13 (m, 2H), 7.01-6.93 (m, 1H), 3.75 (ddt, 1H), 3.58-3.48 (m, 1H), 3.12-2.92 (m, 4H), 2.88-2.78 (m, 1H), 2.26-2.15 (m, 1H), 2.02-1.78 (m, 3H), 1.70-1.57 (m, 1H).

Step 2. The title compound was prepared from the foregoing compound and Intermediate 8 by application of General Procedure F. Data for the title compound: LCMS (Method 6, Rt 8.02 min). M+=501. ¹H NMR (400 MHz, CH₃OH-d₄): δ 7.47 (s, 1H), 7.37-7.27 (m, 12H), 7.24-7.19 (m, 2H), 7.05 (tdd, 1H), 4.59 (s, 2H), 3.96-3.89 (m, 2H), 3.89-3.84 (m, 1H), 3.55-3.32 (m, 3H), 3.26-3.23 (m, 1H), 2.43-2.34 (m, 1H), 2.26-2.22 (m, 1H), 2.16-2.07 (m, 1H), 2.07-2.01 (m, 1H), 2.01-1.88 (m, 1H).

### Example 99

### (R)-3-(3,4-Difluoro-phenylsulfanyl)-1-[2-(hydroxy-diphenyl-methyl)-oxazol-5-ylmethyl]-1-azonia-bicyclo[2.2.2]octane; bromide

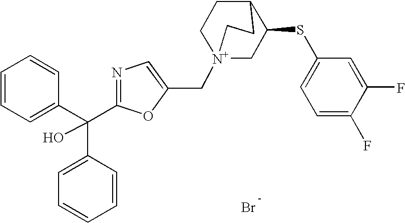

Step 1. (R)-3-(3,4-Difluoro-phenylsulfanyl)-1-aza-bicyclo[2.2.2]octane was prepared from methanesulfonic acid (S)-(1-aza-bicyclo[2.2.2]oct-3-yl) ester and 3,4-difluoro benzenethiol by analogy to the procedure described in General Procedure C2. ¹H NMR (300 MHz, CHCl₃-d): δ 7.27-7.19 (m, 1H), 7.19-7.07 (m, 2H), 3.57-3.41 (m, 2H), 3.08-2.72 (m, 5H), 2.27-2.13 (m, 1H), 1.99-1.82 (m, 2H), 1.80-1.53 (m, 2H).

Step 2. The title compound was prepared from the foregoing compound and Intermediate 8 by application of General Procedure F. Data for the title compound: LCMS (Method 6, Rt 8.20 min). M+=519. ¹H NMR (400 MHz, CH₃OH-d₄): δ 7.48-7.41 (m, 2H), 7.36-7.25 (m, 12H), 4.59 (s, 2H), 3.85 (d, 2H), 3.51-3.33 (m, 5H), 2.41-2.39 (m, 1H), 2.22 (d, 1H), 2.13-2.10 (m, 1H), 2.05-1.91 (m, 2H).

### Example 100

### (R)-1-[2-((R)—Cyclohexyl-hydroxy-phenyl-methyl)-oxazol-5-ylmethyl]-3-phenylsulfanylmethyl-1-azonia-bicyclo[2.2.2]octane; bromide

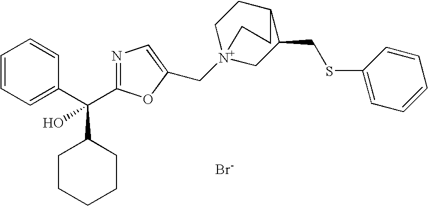

Step 1. (R)-3-phenylsulfanylmethyl-1-aza-bicyclo[2.2.2]octane was prepared from methanesulfonic acid (R)-1-(1-aza-bicyclo[2.2.2]oct-3-yl)methyl ester (D)-tartrate salt and thiophenol by analogy to the procedure described in Step 1 Example 43. ¹H NMR (400 MHz, CDCl₃): δ 7.39-7.23 (4H, m), 7.20-7.12 (1H, m), 3.18-3.07 (1H, m), 3.04-2.91 (2H, m), 2.89-2.70 (4H, m), 2.52-2.43 (1H, m), 1.90-1.80 (2H, m), 1.74-1.59 (2H, m), 1.55-1.35 (2H, m).

Step 2. The title compound was prepared from the foregoing compound and Intermediate 14 by application of General Procedure F. Data for the title compound: LCMS (Method 6, Rt 8.72 min). M+=503 ¹H NMR (400 MHz, CHCl₃-d): δ 7.58 (d, 2H), 7.48 (s, 1H), 7.39-7.26 (m, 7H), 7.23 (d, 1H), 5.23-5.03 (m, 2H), 4.10 (d, 1H), 3.78 (d, 3H), 3.69 (s, 1H), 3.55 (s, 1H), 3.34-3.27 (m, 1H), 2.99-2.90 (m, 2H), 2.32 (s, 1H), 2.22 (s, 2H), 2.10-1.98 (m, 2H), 1.86 (s, 2H), 1.73 (d, 1H), 1.67 (m, 3H), 1.36-1.27 (m, 3H), 1.18-1.06 (m, 3H).

### Example 101

### (R)-1-[3-(Cyclohexyl-hydroxy-phenyl-methyl)-isoxazol-5-ylmethyl]-3-(3-fluoro-phenoxy)-1-azonia-bicyclo[2.2.2]octane; chloride

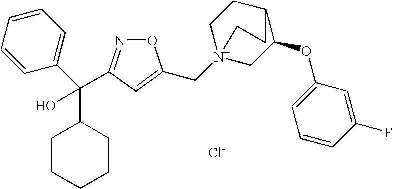

The title compound was prepared from Intermediate 24 and Intermediate 21, Enantiomer 2 by application of General Procedure F. Data for the title compound LCMS (Method 6, Rt 8.70 min). M+=491. ¹H NMR (400 MHz, DMSO-d₆): δ 7.49 (dd, 2H), 7.40-7.29 (m, 3H), 7.25-7.20 (m, 1H), 6.93-6.79 (m, 4H), 5.90 (s, 1H), 4.96 (s, 1H), 4.77 (s, 2H), 3.95 (dd, 1H), 3.49 (d, 4H), 2.43 (s, 1H), 2.26-2.10 (m, 2H), 2.07-1.98 (m, 1H), 1.95-1.82 (m, 2H), 1.69 (d, 1H), 1.59 (s, 4H), 1.28-1.14 (m, 3H), 1.10-0.98 (m, 3H).

### Example 102

### (R)-1-[2-(Hydroxy-diphenyl-methyl)-oxazol-5-ylmethyl]-3-(thiophen-2-yloxy)-1-azonia-bicyclo[2.2.2]octane; bromide

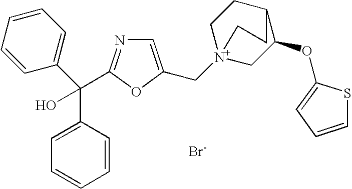

The title compound was prepared from Intermediate 32 and Intermediate 8 by application of General Procedure F. Data for the title compound LCMS (Method 6, Rt 7.38 min). M+=473. ¹H NMR (400 MHz, DMSO-d₆): δ 7.54 (s, 1H), 7.39-7.24 (m, 10H), 7.12 (s, 1H), 6.87 (dd, 1H), 6.77 (dd, 1H), 6.41 (dd, 1H), 4.76-4.70 (m, 3H), 3.84 (ddd, 1H), 3.56-3.34 (m, 6H), 2.16-1.94 (m, 2H), 1.87 (d, 2H).

### Example 103

### (R)-1-[3-(Hydroxy-diphenyl-methyl)-[1,2,4]oxadiazol-5-ylmethyl]-3-phenoxy-1-azonia-bicyclo[2.2.2]octane; chloride

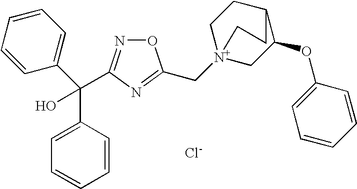

The title compound was prepared from R-3-phenoxyquinuclidine and Intermediate 22 by application of General Procedure F. Data for the title compound LCMS (Method 6, Rt 7.76 min). M+=468. ¹H NMR (400 MHz, DMSO-d₆): δ 7.39-7.26 (m, 12H), 7.13 (s, 1H), 7.03-6.98 (m, 1H), 6.97-6.94 (m, 2H), 5.05 (s, 2H), 4.93 (s, 1H), 4.13-4.04 (m, 1H), 3.60 (dd, 5H), 2.45 (s, 1H), 2.19 (s, 1H), 2.10-2.01 (m, 1H), 2.00-1.83 (m, 2H).

### Example 104

### (R)-3-(3-Chloro-4-methyl-phenoxy)-1-[3-(hydroxy-diphenyl-methyl)-[1,2,4]oxadiazol-5-ylmethyl]-1-azonia-bicyclo[2.2.2]octane; chloride

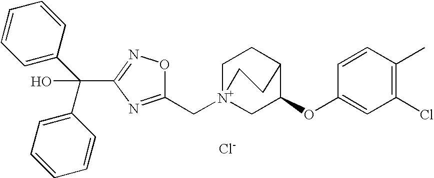

The title compound was prepared from Intermediate 22 and the compound of Step 1, Example 59 by application of General Procedure F. Data for the title compound: LCMS (Method 6, Rt 8.57 min). M+=516. ¹H NMR (400 MHz, DMSO-d₆): δ 7.39-7.34 (m, 4H), 7.34-7.25 (m, 7H), 7.12 (s, 1H), 7.08 (d, 1H), 6.88 (dd, 1H), 5.04 (s, 2H), 4.95 (s, 1 H), 4.11-4.02 (m, 1H), 3.70-3.50 (m, 5H), 2.44 (s, 1H), 2.27 (s, 3H), 2.15 (s, 1H), 2.10-1.99 (m, 1H), 1.98-1.83 (m, 2H).

### Example 105

### (R)-1-[2-((R)—Cyclohexyl-hydroxy-phenyl-methyl)-oxazol-5-ylmethyl]-3-(3-fluoro-phenylsulfanyl)-1-azonia-bicyclo[2.2.2]octane; bromide

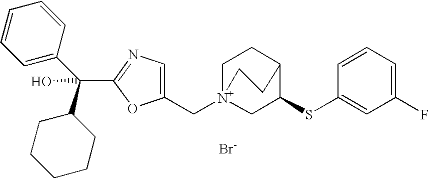

The title compound was prepared from Intermediate 14 and the compound of Step 1, Example 98 by application of General Procedure F. Data for the title compound: LCMS (Method 6, Rt 8.62 min). M+=507. ¹H NMR (400 MHz, DMSO-d₆): δ 7.50-7.37 (m, 4H), 7.34-7.19 (m, 5H), 7.17-7.11 (m, 1H), 6.07 (s, 1H), 4.67-4.56 (m, 2H), 4.10 (t, 1H), 3.96-3.88 (m, 1H), 3.53-3.31 (m, 4H), 3.30-3.23 (m, 1H), 2.24 (t, 1H), 2.16 (s, 2H), 2.02 (d, 2H), 1.94-1.85 (m, 1H), 1.69 (s, 1H), 1.65-1.50 (m, 3H), 1.29-0.89 (m, 6H).

### Example 106

### (R)-1-[2-((R)—Cyclohexyl-hydroxy-phenyl-methyl)-oxazol-5-ylmethyl]-3-(4-fluoro-phenylsulfanylmethyl)-1-azonia-bicyclo[2.2.2]octane; bromide

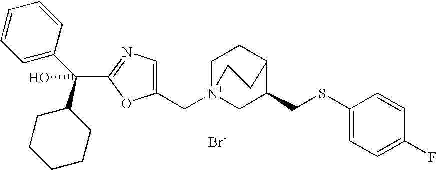

Step 1. (R)-3-(4-Fluoro-phenylsulfanylmethyl-1-aza-bicyclo[2.2.2]octane was prepared from methanesulfonic acid (R)-1-(1-aza-bicyclo[2.2.2]oct-3-yl)methyl ester (D)-tartrate salt and 4-fluorothiophenol by analogy to the procedure described in Step 1 Example 43. ¹H NMR (400 MHz, CDCl₃): δ 7.39-7.30 (2H, m), 7.05-6.93 (2H, m), 3.15-3.03 (1H, m), 3.00-2.70 (6H, m), 2.49-2.40 (1H, m), 1.89-1.74 (2H, m), 1.72-1.58 (2H, m), 1.55-1.35 (2H, m).

Step 2. The title compound was prepared from the foregoing compound and Intermediate 14 by application of General Procedure F. Data for the title compound: LCMS (Method 6, Rt 8.81 min). M+=521. ¹H NMR (400 MHz, CHCl₃-d): δ 7.60-7.55 (m, 2H), 7.48 (s, 1H), 7.42-7.35 (m, 2H), 7.31 (t, 2H), 7.22 (t, 1H), 7.06-6.99 (m, 2H), 5.20-5.07 (m, 2H), 4.16-4.08 (m, 1H), 3.87 (t, 1H), 3.73 (d, 3H), 3.63-3.54 (m, 1H), 3.38 (dd, 1H), 2.97-2.84 (m, 2H), 2.32 (s, 1H), 2.21 (d, 2H), 2.01 (s, 2H), 1.86 (s, 2H), 1.73 (d, 1H), 1.65 (d, 4H), 1.35-1.24 (m, 3H), 1.17-1.07 (m, 2H).

### Example 107

### (R)-3-(4-Chloro-phenylsulfanylmethyl)-1-[2-(R)cyclohexyl-hydroxy-phenyl-methyl)-oxazol-5-ylmethyl]-1-azonia-bicyclo[2.2.2]octane; bromide

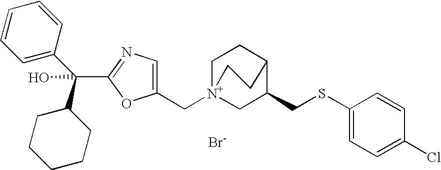

Step 1. (R)-3-(4-Chloro-phenylsulfanylmethyl-1-aza-bicyclo[2.2.2]octane was prepared from methanesulfonic acid (R)-1-(1-aza-bicyclo[2.2.2]oct-3-yl)methyl ester (D)-tartrate salt and 4-chlorothiophenol by analogy to the procedure described in Step 1 Example 43. ¹H NMR (400 MHz, CDCl₃): δ 7.30-7.20 (4H, m), 3.15-3.05 (1H, m), 3.03-2.70 (6H, m), 2.50-2.38 (1H, m), 1.89-1.78 (2H, m), 1.73-1.59 (2H, m), 1.55-1.35 (2H, m).

Step 2. The title compound was prepared from the foregoing compound and Intermediate 14 by application of General Procedure F. Data for the title compound: LCMS (Method 6, Rt 9.17 min). M+=537. ¹H NMR (400 MHz, CHCl₃-d): δ 7.59-7.54 (m, 2H), 7.47 (s, 1H), 7.32-7.25 (m, 6H), 7.24-7.18 (m, 1H), 5.15-5.04 (m, 2H), 4.19 (s, 1H), 3.90-3.66 (m, 4H), 3.57 (d, 1H), 3.41 (dd, 1H), 3.02-2.88 (m, 2H), 2.31 (s, 2H), 2.28-2.15 (m, 3H), 2.02 (d, 2H), 1.85 (s, 2H), 1.72 (d, 1H), 1.36-1.23 (m, 4H), 1.19-1.04 (m, 3H).

### Example 108

### (R)-1-[2-((R)—Cyclohexyl-hydroxy-phenyl-methyl)-oxazol-5-ylmethyl]-3-(3-fluoro-phenylsulfanylmethyl)-1-azonia-bicyclo[2.2.2]octane; bromide

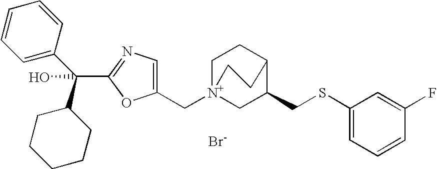

Step 1. (R)-3-(3-Fluoro-phenylsulfanylmethyl-1-aza-bicyclo[2.2.2]octane was prepared from methanesulfonic acid (R)-1-(1-aza-bicyclo[2.2.2]oct-3-yl)methyl ester (D)-tartrate salt and 3-fluorothiophenol by analogy to the procedure described in Step 1 Example 43. ¹H NMR (400 MHz, CDCl₃): δ 7.28-7.16 (1H, m), 7.10-6.95 (2H, m), 6.89-6.80 (1H, m), 3.18-3.08 (1H, m), 3.05-2.91 (2H, m), 2.90-2.72 (4H, m), 2.52-2.43 (1H, m), 1.91-1.80 (2H, m), 1.75-1.60 (2H, m), 1.56-1.35 (2H, m).

Step 2. The title compound was prepared from the foregoing compound and Intermediate 14 by application of General Procedure F. Data for the title compound: LCMS (Method 6, Rt 8.83 min). M+=521. ¹H NMR (400 MHz, CHCl₃-d): δ 7.59-7.54 (m, 2H), 7.51-7.45 (m, 1H), 7.37-7.21 (m, 3H), 7.26-7.18 (m, 1H), 7.10 (ddd, 1H), 7.03-6.98 (m, 1H), 6.91 (tdd, 1H), 5.17-5.04 (m, 2H), 4.19 (d, 1H), 3.87-3.71 (m, 4H), 3.62-3.52 (m, 1H), 3.44-3.36 (m, 1H), 3.06-2.91 (m, 2H), 2.27 (d, 3H), 2.03 (d, 3H), 1.87 (s, 3H), 1.72 (d, 1H), 1.37-1.23 (m, 4H), 1.20-1.04 (m, 3H).

### Example 109

### (S)-1-[2-((R)—Cyclohexyl-hydroxy-phenyl-methyl)-oxazol-5-ylmethyl]-3-(4-fluoro-phenylsulfanylmethyl)-1-azonia-bicyclo[2.2.2]octane; bromide

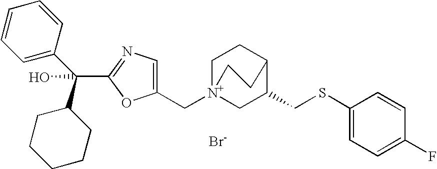

Step 1. (S)-3-(4-Fluoro-phenylsulfanylmethyl-1-aza-bicyclo[2.2.2]octane was prepared from methanesulfonic acid (S)-1-(1-aza-bicyclo[2.2.2]oct-3-yl)methyl ester (L)-tartrate salt and 4-fluorothiophenol by analogy to the procedure described in Step 1 Example 43. ¹H NMR (400 MHz, CDCl₃): δ 7.39-7.30 (2H, m), 7.05-6.95 (2H, m), 3.15-3.03 (1H, m), 2.98-2.70 (6H, m), 2.50-2.41 (1H, m), 1.89-1.74 (2H, m), 1.70-1.59 (2H, m), 1.55-1.35 (2H, m).

Step 2. The title compound was prepared from the foregoing compound and Intermediate 14 by application of General Procedure F. Data for the title compound: LCMS (Method 6, Rt 8.82 min). M+=521. ¹H NMR (400 MHz, CHCl₃-d): δ 7.61-7.54 (m, 2H), 7.47 (d, 1H), 7.42-7.36 (m, 2H), 7.30 (t, 2H), 7.21 (t, 1H), 7.06-6.97 (m, 2H), 5.19 (d, 1H), 5.09 (d, 1H), 3.92-3.73 (m, 3H), 3.69 (s, 1H), 3.55 (d, 1H), 3.34 (dd, 1H), 2.95-2.87 (m, 2H), 2.33 (s, 1H); 2.18 (s, 2H), 2.02 (d, 3H), 1.85 (s, 3H), 1.73 (d, 2H), 1.36-1.19 (m, 4H), 1.22-1.06 (m, 3H).

### Example 110

### (S)-3-(4-Chloro-phenylsulfanylmethyl)-1-[2-((R)cyclohexyl-hydroxy-phenyl-methyl)-oxazol-5-ylmethyl]-1-azonia-bicyclo[2.2.2]octane; bromide

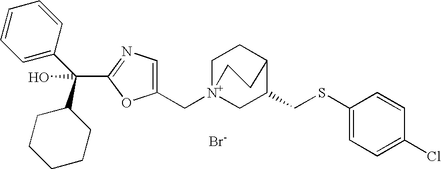

Step 1. (S)-3-(4-Chloro-phenylsulfanylmethyl-1-aza-bicyclo[2.2.2]octane was prepared from methanesulfonic acid (S)-1-(1-aza-bicyclo[2.2.2]oct-3-yl)methyl ester (L)-tartrate salt and 4-chlorothiophenol by analogy to the procedure described in Step 1 Example 43. ¹H NMR (400 MHz, CDCl₃): δ 7.30-7.20 (4H, m), 3.17-3.09 (1H, m), 3.02-2.60 (6H, m), 2.50-2.42 (1H, m), 1.90-1.79 (2H, m), 1.72-1.60 (2H, m), 1.55-1.35 (2H, m).

Step 2. The title compound was prepared from the foregoing compound and Intermediate 14 by application of General Procedure F. Data for the title compound: LCMS (Method 6, Rt 9.19 min). M+=537. ¹H NMR (400 MHz, CHCl₃-d): δ 7.56 (d, 2H), 7.46 (s, 1H), 7.29 (dd, 6H), 7.21 (s, 1H), 5.15 (d, 1H), 5.07 (d, 1H), 3.80 (d, 3H), 3.68 (s, 2H), 3.52 (d, 1H), 3.37 (d, 1H), 2.99-2.88 (m, 2H), 2.32 (s, 2H), 2.19 (s, 2H), 2.09-1.94 (m, 2H), 1.85 (s, 2H), 1.73 (d, 2H), 1.37-1.21 (m, 4H), 1.19-1.08 (m, 3H).

### Example 111

### (S)-1-[2-((R)—Cyclohexyl-hydroxy-phenyl-methyl)-oxazol-5-ylmethyl]-3-(3-fluoro-phenylsulfanylmethyl)-1-azonia-bicyclo[2.2.2]octane; bromide

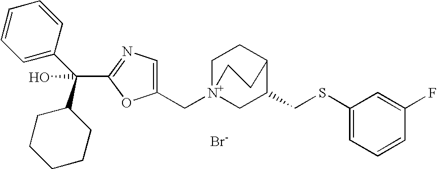

Step 1. (S)-3-(3-Fluoro-phenylsulfanylmethyl-1-aza-bicyclo[2.2.2]octane was prepared from methanesulfonic acid (S)-1-(1-aza-bicyclo[2.2.2]oct-3-yl)methyl ester (L)-tartrate salt and 3-fluorothiophenol by analogy to the procedure described in Step 1 Example 43. ¹H NMR (400 MHz, CDCl₃): δ 7.29-7.20 (1H, m), 7.10-7.05 (1H, m), 7.03-7.00 (1H, m), 6.89-6.82 (1H, m), 3.20-3.10 (1H, m), 3.05-2.92 (2H, m), 2.90-2.73 (4H, m), 2.51-2.44 (1H, m), 1.90-1.80 (2H, m), 1.73-1.60 (2H, m), 1.56-1.38 (2H, m).

Step 2. The title compound was prepared from the foregoing compound and Intermediate 14 by application of General Procedure F. Data for the title compound: LCMS (Method 6, Rt 8.89 min). M+=521. ¹H NMR (400 MHz, CHCl₃-d): δ 7.56-7.51 (m, 2H), 7.45 (s, 1H), 7.27 (t, 3H), 7.24-7.16 (m, 1H), 7.10-7.03 (m, 1H), 7.01-6.96 (m, 1H), 6.89 (td, 1H), 5.08 (q, 2H), 3.78 (t, 4H), 3.70 (s, 1H), 3.48 (d, 1H), 3.34 (dd, 1H), 3.02-2.89 (m, 2H), 2.30 (s, 2H), 2.27-2.14 (m, 3H), 2.00 (d, 2H), 1.84 (d, 2H), 1.70 (d, 1H), 1.33-1.16 (m, 4H), 1.17-1.02 (m, 3H).

### Example 112

### (R)-1-[2-((R)—Cyclohexyl-hydroxy-phenyl-methyl)-oxazol-5-ylmethyl]-3-(2-methyl-allyloxy)-1-azonia-bicyclo[2.2.2]octane; bromide

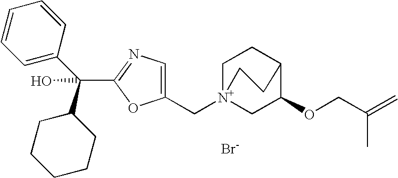

Step 1. (R)-3-(2-Methyl-allyloxy)-1-aza-bicyclo[2.2.2]octane was prepared from 3-(R)-quinuclidinol and 3-bromo-2-methyl-propene by application of General Procedure A. LCMS (Method 7, Rt 1.82 min). MH⁺=182.12.

Step 2. The title compound was prepared from the foregoing compound and Intermediate 14 by application of General Procedure F. Data for the title compound: LCMS (Method 6, Rt 8.15 min). M+=451. ¹H NMR (400 MHz, DMSO-d₆): δ 7.48 (d, 3 H), 7.37-7.29 (m, 2H), 7.27-7.21 (m, 1H), 6.09 (s, 1H), 4.94 (dd, 1H), 4.87 (d, 1H), 4.68-4.58 (m, 2H), 3.93-3.76 (m, 3H), 3.64-3.56 (m, 1H), 3.40 (t, 1H), 3.30-3.13 (m, 4H), 2.33 (s, 1H), 2.26 (t, 1H), 2.02-1.89 (m, 2H), 1.85-1.47 (m, 6H), 1.40 (s, 3H), 1.28-0.91 (m, 6H).

### Example 113

### (R)-3-[((E)-But-2-enyl)oxy]-1-[2-((R)-cyclohexyl-hydroxy-phenyl-methyl)-oxazol-5-ylmethyl]-1-azonia-bicyclo[2.2.2]octane; bromide

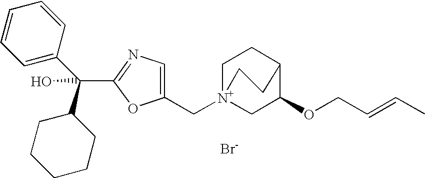

Step 1. (R)-3-[((E)-But-2-enyl)oxy]-1-aza-bicyclo[2.2.2]octane was prepared from 3-(R)-quinuclidinol and (E)-1-bromo-but-2-ene by application of General Procedure A. LCMS (Method 7, Rt 1.93 min). MH⁺=182.12.

Step 2. The title compound was prepared from the foregoing compound and Intermediate 14 by application of General Procedure F. Data for the title compound: LCMS (Method 6, Rt 8.05 min). M+=451. ¹H NMR (400 MHz, DMSO-d₆): δ 7.45-7.40 (m, 3H), 7.29 (t, 2H), 7.23-7.16 (m, 1H), 6.06-6.01 (m, 1H), 5.69-5.57 (m, 1H), 5.50-5.41 (m, 1H), 4.63-4.53 (m, 2H), 3.90-3.78 (m, 3H), 3.59-3.50 (m, 1H), 3.40-3.27 (m, 1H), 3.25-3.16 (m, 3H), 3.15-3.05 (m, 1H), 2.31-2.21 (m, 1H), 2.20 (d, 1H), 1.97-1.83 (m, 2H), 1.71-1.48 (m, 9H), 1.27-0.89 (m, 6H).

### Example 114

### (R)-1-[2-(Hydroxy-diphenyl-methyl)-oxazol-5-ylmethyl]-3-(thiophen-3-yloxy)-1-azonia-bicyclo[2.2.2]octane; bromide

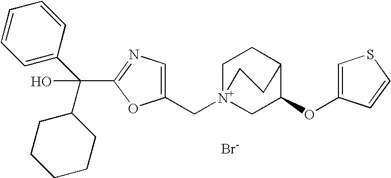

The title compound was prepared from Intermediate 8 and Intermediate 33 by application of General Procedure F. Data for the title compound: LCMS (Method 6, Rt 7.40 min). M+=473. ¹H NMR (400 MHz, DMSO-d₆): δ 7.53 (s, 1H), 7.48 (dd, 1H), 7.39-7.30 (m, 8H), 7.30-7.23 (m, 3H), 7.11 (s, 1H), 6.81 (dd, 1H), 6.64 (dd, 1H), 4.76-4.68 (m, 3H), 3.84 (dd, 1H), 3.50-3.31 (m, 5H), 2.45 (s, 1H), 2.12-1.94 (m, 1H), 1.92-1.79 (m, 2H).

### Example 115

### (R)-1-[3-(Hydroxy-diphenyl-methyl)-[1,2,4]oxadiazol-5-ylmethyl]-3-(thiophen-3-yloxy)-1-azonia-bicyclo[2.2.2]octane; chloride

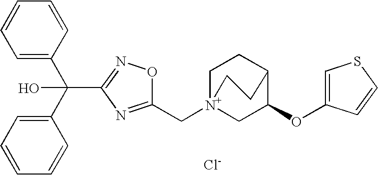

The title compound was prepared from Intermediate 22 and Intermediate 33 by application of General Procedure F. Data for the title compound: LCMS (Method 6, Rt 7.77 min). M+=474. ¹H NMR (400 MHz, DMSO-d₆): δ 7.50 (dd, 1H), 7.40-7.26 (m, 10H), 7.15 (s, 1H), 6.82 (dd, 1H), 6.68 (dd, 1H), 5.05 (s, 2H), 4.78 (s, 1H), 4.13-4.04 (m, 1H), 3.73-3.49 (m, 6H), 2.19-1.97 (m, 2H), 1.98-1.82 (m, 2H).

### Example 116

### (R)-1-[3-(Hydroxy-diphenyl-methyl)-[1,2,4]oxadiazol-5-ylmethyl]-3-(thiophen-2-yloxy)-1-azonia-bicyclo[2.2.2]octane; chloride

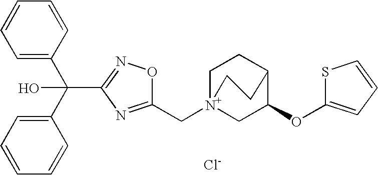

The title compound was prepared from Intermediate 22 and Intermediate 32 by application of General Procedure F. Data for the title compound: LCMS (Method 6, Rt 7.65 min). M+=474. ¹H NMR (400 MHz, DMSO-d₆): δ 7.40-7.26 (m, 10H), 7.16 (s, 1H), 6.87 (dd, 1H), 6.77 (dd, 1H), 6.42 (dd, 1H), 5.11-5.02 (m, 2H), 4.78 (s, 1H), 4.09 (ddd, 1H), 3.79-3.46 (m, 6H), 2.15 (d, 1H), 2.10-1.99 (m, 1H), 1.96-1.84 (m, 2H).

### Example 117

### (R)-3-(3-Chloro-phenoxy)-1-[3-(hydroxy-diphenyl-methyl)-[1,2,4]oxadiazol-5-ylmethyl]-1-azonia-bicyclo[2.2.2]octane; chloride

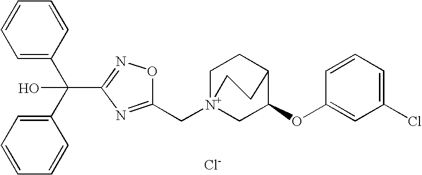

The title compound was prepared from Intermediate 22 and the compound of Step 1, Example 62 by application of General Procedure F. Data for the title compound: LCMS (Method 6, Rt 8.26 min). M+=502. ¹H NMR (400 MHz, DMSO-d₆): δ 7.40-7.26 (m, 11H), 7.13 (s, 1H), 7.09-7.06 (m, 2H), 6.97-6.93 (m, 1H), 5.10-4.96 (m, 2H), 4.14-4.05 (m, 1H), 3.72-3.50 (m, 6H), 2.46 (s, 1H), 2.16 (s, 1H), 2.10-2.01 (m, 1H), 1.99-1.82 (m, 2H).

### Example 118

### (R)-1-[3-(Hydroxy-diphenyl-methyl)-[1,2,4]oxadiazol-5-ylmethyl]-3-phenylsulfanyl-1-azonia-bicyclo[2.2.2]octane; chloride

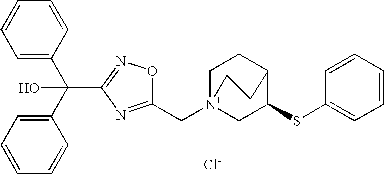

Step 1. (R)-3-(phenylsulfanyl)-1-aza-bicyclo[2.2.2]octane was prepared from methanesulfonic acid (S)-(1-aza-bicyclo[2.2.2]oct-3-yl) ester and thiophenol by analogy to the procedure described in Procedure C2. LCMS (Method 7, Rt 2.17 min). MH⁺=220.1.

Step 2. The title compound was prepared from the foregoing compound and Intermediate 22 by application of General Procedure F. Data for the title compound: LCMS (Method 6, Rt 7.96 min). M+=484. ¹H NMR (DMSO-d₆): δ 7.44-7.26 (15H, m), 7.16-7.11 (1H, m), 4.99 (2H, s), 4.15-4.07 (1H, m), 4.05-3.97 (1H, m), 3.69-3.51 (5H, m), 2.26-2.15 (2H, m), 2.09-1.99 (2H, m), 1.98-1.87 (1H, m).

### Example 119

### (R)-1-[2-((R)—Cyclohexyl-hydroxy-phenyl-methyl)-oxazol-5-ylmethyl]-3-cyclohexylsulfanyl-1-azonia-bicyclo[2.2.2]octane; bromide

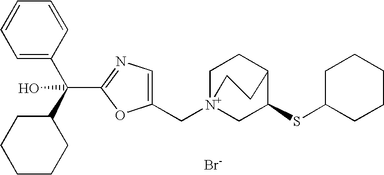

Step 1. (R)-3-(Cyclohexylsulfanyl)-1-aza-bicyclo[2.2.2]octane was prepared from methanesulfonic acid (S)-(1-aza-bicyclo[2.2.2]oct-3-yl) ester and cyclohexyl mercaptan by analogy to the procedure described in Procedure C2. LCMS (Method 7, Rt 2.25 min). MH⁺=226.16.

Step 2. The title compound was prepared from the foregoing compound and Intermediate 14 by application of General Procedure F. Data for the title compound: LCMS (Method 6, Rt 9.03 min). M+=495. ¹H NMR (400 MHz, DMSO-d₆): δ 7.47 (d, 3H), 7.34-7.29 (m, 2H), 7.26-7.21 (m, 1H), 6.07 (s, 1H), 4.64-4.51 (m, 2H), 3.83-3.72 (m, 1H), 3.48-3.34 (m, 4H), 3.31-3.21 (m, 1H), 3.06 (ddd, 1H), 2.78 (td, 1H), 2.24 (t, 1H), 2.12-2.02 (m, 2H), 1.97 (t, 2H), 1.92-1.81 (m, 3H), 1.68 (s, 4H), 1.67-1.50 (m, 5H), 1.37-1.03 (m, 7H), 1.03-0.92 (m, 2H).

### Example 120

### (R)-1-[2-((R)—Cyclohexyl-hydroxy-phenyl-methyl)-oxazol-5-ylmethyl]-3-isobutylsulfanyl-1-azonia-bicyclo[2.2.2]octane; bromide

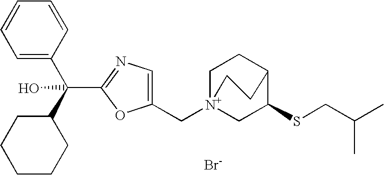

Step 1. (R)-3-(Isobutylsulfanyl)-1-aza-bicyclo[2.2.2]octane was prepared from methanesulfonic acid (S)-(1-aza-bicyclo[2.2.2]oct-3-yl) ester and isobutyl mercaptan by analogy to the procedure described in Procedure C2. LCMS (Method 7, Rt 2.31 min). MH⁺=200.14.

Step 2. The title compound was prepared from the foregoing compound and Intermediate 14 by application of General Procedure F. Data for the title compound: LCMS (Method 6, Rt 8.56 min). M+=469. ¹H NMR (400 MHz, CHCl₃-d): δ 7.60-7.55 (m, 2H), 7.49 (s, 1H), 7.33 (t, 2H), 7.27-7.20 (m, 1H), 5.18-5.02 (m, 2H), 4.49-4.40 (m, 1H), 4.17-4.05 (m, 2H), 3.91-3.82 (m, 1H), 3.81-3.70 (m, 1H), 3.33-3.22 (m, 2H), 2.92 (ddd, 1H), 2.40 (d, 2H), 2.30 (s, 2H), 2.22-2.00 (m, 3H), 1.92-1.81 (m, 1H), 1.77 (dq, 2H), 1.70-1.59 (m, 2H), 1.41-1.09 (m, 7H), 0.97 (dd, 6H).

### Example 121

### (S)-1-[5-((R)—Cyclohexyl-hydroxy-phenyl-methyl)-[1,3,4]oxadiazol-2-ylmethyl]-3-phenylsulfanylmethyl-1-azonia-bicyclo[2.2.2]octane; bromide

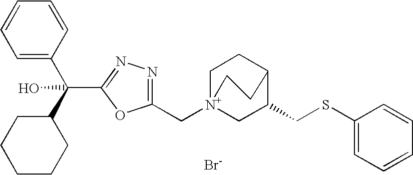

The title compound was prepared from Intermediate 18 and the compound of Step 1, Example 43 by application of General Procedure F. Data for the title compound: LCMS (Method 6, Rt 8.65 min). M+=504. ¹H NMR (400 MHz, CHCl₃-d): δ 7.39-7.27 (m, 6H), 7.26-7.16 (m, 4H), 5.59 (s, 1H), 5.15-5.06 (m, 1H), 4.80 (d, 1H), 4.04-3.83 (m, 4H), 3.70-3.62 (m, 1H), 3.54-3.46 (m, 1H), 3.11-2.96 (m, 2H), 2.37-2.19 (m, 3H), 2.14-2.05 (m, 2H), 1.91-1.71 (m, 4H), 1.85-1.38 (m, 2H), 1.41-1.23 (m, 3H), 1.21-1.01 (m, 3H).

### Example 122

### (S)-1-[2-(Cyclobutyl-hydroxy-phenyl-methyl)-oxazol-5-ylmethyl]-3-phenylsulfanylmethyl-1-azonia-bicyclo[2.2.2]octane; bromide

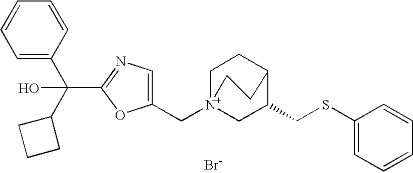

The title compound was prepared from Intermediate 15 and the compound of Step 1, Example 43 by application of General Procedure F. Data for the title compound: LCMS (Method 6, Rt 8.06 min). M+=475. ¹H NMR (400 MHz, CHCl₃-d): δ 7.45 (s, 1H), 7.41-7.29 (m, 6H), 7.28 (d, 1H), 7.26-7.15 (m, 3H), 5.00-4.79 (m, 2H), 3.88-3.66 (m, 4H), 3.52-3.41 (m, 1H), 3.38-3.25 (m, 2H), 3.03-2.94 (m, 2H), 2.41-2.15 (m, 3H), 2.13-1.88 (m, 4H), 1.85-1.70 (m, 5H), 1.64-1.55 (m, 1H).

### Example 123

### (S)-1-[3-(Cyclohexyl-hydroxy-phenyl-methyl)-isoxazol-5-ylmethyl]-3-phenylsulfanylmethyl-1-azonia-bicyclo[2.2.2]octane; chloride

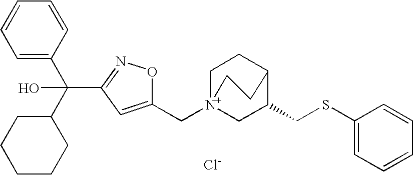

The title compound was prepared from Intermediate 21, Enantiomer 2 and the compound of Step 1, Example 43 by application of General Procedure F. Data for the title compound: LCMS (Method 6, Rt 9.04 min). M+=503. ¹H NMR (400 MHz, CHCl₃-d): δ 7.56-7.51 (m, 2H), 7.37-7.26 (m, 5H), 7.27-7.17 (m, 3H), 7.04 (s, 1H), 5.31-5.13 (m, 1H), 4.38 (s, 1H), 3.94-3.70 (m, 4H), 3.58-3.49 (m, 1H), 3.43 (dd, 1H), 3.06-2.95 (m, 2H), 2.23 (s, 3H), 2.07-1.98 (m, 2H), 1.83 (s, 5H), 1.73 (s, 2H), 1.36-1.05 (m, 6H).

### Example 124

### (R)-1-[3-(Cyclohexyl-hydroxy-phenyl-methyl)-isoxazol-5-ylmethyl]-3-phenylsulfanylmethyl-1-azonia-bicyclo[2.2.2]octane; chloride

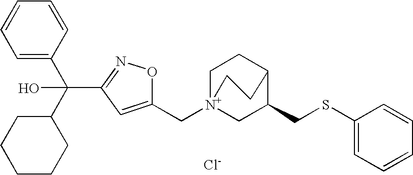

The title compound was prepared from Intermediate 21, Enantiomer 2 and the compound of Step 1, Example 100 by application of General Procedure F. Data for the title compound: LCMS (Method 6, Rt 9.07 min). M+=503. ¹H NMR (400 MHz, CHCl₃-d): δ 7.54 (d, 2H), 7.37-7.25 (m, 6H), 7.28-7.16 (m, 2H), 7.04 (s, 1H), 5.35 (d, 1H), 5.09 (d, 1H), 3.96-3.70 (m, 5H), 3.56 (s, 1H), 3.45 (s, 1H), 3.07-2.94 (m, 2H), 2.23 (s, 4H), 2.01 (s, 2H), 1.84 (s, 2H), 1.73 (s, 1H), 1.35-1.21 (m, 4H), 1.19-1.01 (m, 4H).

### Example 125

### (S)-1-[3-(Cyclohexyl-hydroxy-phenyl-methyl)-isoxazol-5-ylmethyl]-3-(4-fluoro-phenylsulfanylmethyl)-1-azonia-bicyclo[2.2.2]octane; chloride

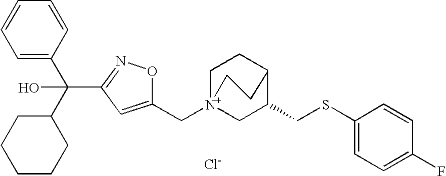

The title compound was prepared from Intermediate 21, Enantiomer 2 and the compound of Step 1, Example 109 by application of General Procedure F. Data for the title compound: LCMS (Method 6, Rt 9.15 min). M+=521. ¹H NMR (400 MHz, CHCl₃-d): δ 7.52 (d, 2H), 7.37 (dd, 2H), 7.28 (d, 2H), 7.19 (t, 1H), 7.03-6.97 (m, 3H), 5.27 (d, 1H), 5.17 (d, 1H), 3.91 (t, 1H), 3.81 (d, 2H), 3.69 (s, 1H), 3.59 (d, 1H), 3.44 (d, 1H), 2.97-2.88 (m, 2H), 2.22 (d, 4H), 2.06-1.96 (m, 3H), 1.85 (s, 3H), 1.72 (s, 1H), 1.63 (s, 1H), 1.32-1.13 (m, 3H), 1.15-1.03 (m, 3H).

### Example 126

### (R)-3-Benzylsulfanyl-1-[3-(cyclohexyl-hydroxy-phenyl-methyl)-isoxazol-5-ylmethyl]-1-azonia-bicyclo[2.2.2]octane; chloride

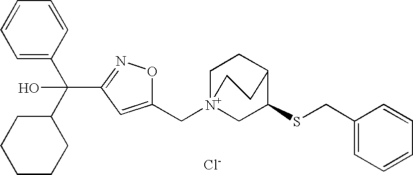

The title compound was prepared from Intermediate 21, Enantiomer 2 and (R)-(3-benzylsulfanyl-1-aza-bicyclo[2.2.2]octane (General Procedure C2) by application of General Procedure F. Data for the title compound: LCMS (Method 6, Rt 9.09 min). M+=503. ¹H NMR (400 MHz, CHCl₃-d): δ 7.55-7.50 (m, 2H), 7.34-7.25 (m, 7H), 7.19 (d, 1H), 7.03-6.97 (m, 1H), 5.26 (d, 1H), 5.00 (d, 1H), 4.22-4.13 (m, 2H), 3.85-3.71 (m, 4H), 3.44-3.36 (m, 1H), 3.19-3.12 (m, 1H), 3.06 (dd, 1H), 2.26 (d, 3H), 2.04-1.92 (m, 2H), 1.85 (d, 2H), 1.76 (s, 4H), 1.36-1.22 (m, 3H), 1.14-1.04 (m, 3H).

### Example 127

### (R)-3-Benzylsulfanyl-1-[3-(cyclohexyl-hydroxy-phenyl-methyl)-[1,2,4]oxadiazol-5-ylmethyl]-1-azonia-bicyclo[2.2.2]octane; chloride

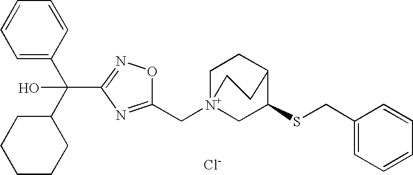

The title compound was prepared from Intermediate 20, Enantiomer 2 and (R)-(3-benzylsulfanyl-1-aza-bicyclo[2.2.2]octane (General Procedure C2) by application of General Procedure F. Data for the title compound: LCMS (Method 6, Rt 8.89 min). M+=504. ¹H NMR (400 MHz, CHCl₃-d): δ 7.48-7.42 (m, 2H), 7.35-7.24 (m, 5H), 7.25-7.17 (m, 3H), 5.56 (d, 1H), 5.04 (d, 1H), 4.60 (s, 1H), 4.31-4.19 (m, 1H), 4.16 (s, 1H), 4.16-3.96 (m, 2H), 3.76 (s, 2H), 3.51 (s, 1H), 3.26 (d, 2H), 2.26 (s, 3H), 2.09-1.99 (m, 2H), 1.92 (d, 2H), 1.76 (d, 1H), 1.62 (d, 2H), 1.39-1.18 (m, 3H), 1.20-1.06 (m, 3H).

### Example 128

### (R)-1-[3-(Cyclohexyl-hydroxy-phenyl-methyl)-[1,2,4]oxadiazol-5-ylmethyl]-3-(3-fluoro-phenoxy)-1-azonia-bicyclo[2.2.2]octane; chloride

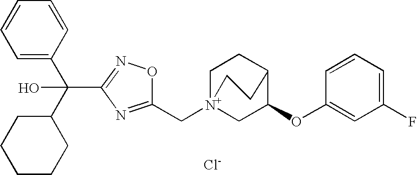

The title compound was prepared from Intermediate 20, Enantiomer 2 and Intermediate 24 by application of General Procedure F. Data for the title compound: LCMS (Method 6, Rt 8.62 min). M+=492. ¹H NMR (400 MHz, CHCl₃-d): δ 7.46-7.41 (m, 2H), 7.31-7.25 (m, 1H), 7.29-7.15 (m, 3H), 6.74 (td, 1H), 6.68-6.59 (m, 2H), 5.66 (d, 1H), 5.09 (d, 1H), 4.86 (s, 2H), 4.74-4.64 (m, 1H), 4.48-4.31 (m, 1H), 4.09 (t, 2H), 3.63-3.47 (m, 2H), 2.49 (s, 1H), 2.34-2.15 (m, 2H), 2.11-1.94 (m, 2H), 1.71 (s, 2H), 1.62 (d, 3H), 1.35-1.14 (m, 3H), 1.16-1.05 (m, 3H).

### Example 129

### (R)-3-(3-Chloro-4-methyl-phenoxy)-1-[3-(cyclohexyl-hydroxy-phenyl-methyl)-[1,2,4]oxadiazol-5-ylmethyl]-1-azonia-bicyclo[2.2.2]octane; chloride

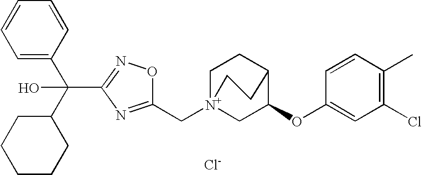

The title compound was prepared from Intermediate 20, Enantiomer 2 and the compound of Step 1, Example 59 by application of General Procedure F. Data for the title compound: LCMS (Method 6, Rt 9.31 min). M+=522. ¹H NMR (400 MHz, CHCl₃-d): δ 7.44 (d, 2H), 7.27-7.12 (m, 4H), 6.90 (d, 1H), 6.70 (dd, 1H), 5.68 (d, 1H), 5.08 (d, 1H), 4.83-4.74 (m, 2H), 4.68-4.59 (m, 1H), 4.43 (s, 1H), 4.11-4.02 (m, 2H), 3.63-3.45 (m, 2H), 2.48 (s, 1H), 2.31 (s, 3H), 2.28-2.15 (m, 2H), 2.08 (s, 2H), 2.05-1.91 (m, 1H), 1.74 (s, 1H), 1.69-1.55 (m, 4H), 1.34-1.22 (m, 3H), 1.24-1.08 (m, 2H).

### Example 130

### (R)-1-[3-(Cyclohexyl-hydroxy-phenyl-methyl)-[1,2,4]oxadiazol-5-ylmethyl]-3-(4-fluoro-benzyloxy)-1-azonia-bicyclo[2.2.2]octane; chloride

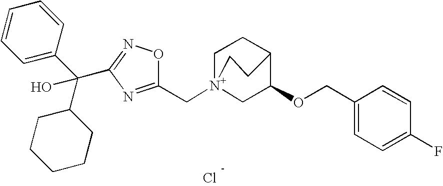

The title compound was prepared from Intermediate 20, Enantiomer 2 and the compound of Step 1, Example 32 by application of General Procedure F. Data for the title compound: LCMS (Method 6, Rt 8.76 min). M+=506. ¹H NMR (400 MHz, CHCl₃-d): δ 7.44-7.39 (m, 2H), 7.30-7.24 (m, 2H), 7.27-7.13 (m, 3H), 7.08-6.99 (m, 2H), 5.60 (d, 1H), 4.95 (d, 1H), 4.72 (s, 1H), 4.47 (d, 2H), 4.36-4.23 (m, 2H), 4.02 (d, 4H), 3.51-3.37 (m, 2H), 2.35 (s, 1H), 2.26-2.16 (m, 2H), 2.09-1.89 (m, 3H), 1.73 (d, 1H), 1.61 (s, 2H), 1.39-1.21 (m, 3H), 1.17-1.04 (m, 3H).

### Example 131

### (R)-1-[3-(Cyclohexyl-hydroxy-phenyl-methyl)-[1,2,4]oxadiazol-5-ylmethyl]-3-phenylsulfanyl-1-azonia-bicyclo[2.2.2]octane; chloride

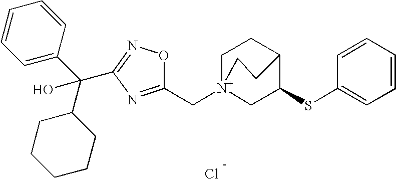

The title compound was prepared from Intermediate 20, Enantiomer 2 and the compound of Step 1, Example 118 by application of General Procedure F. Data for the title compound: LCMS (Method 6, Rt 8.76 min). M+=490. ¹H NMR (400 MHz, CHCl₃-d): δ 7.49-7.37 (m, 4H), 7.42-7.21 (m, 3H), 7.22 (dd, 3H), 5.65 (d, 1H), 5.17 (d, 1H), 4.56 (d, 2H), 4.36 (s, 1H), 4.14 (s, 1H), 4.04 (d, 1H), 3.83 (s, 1H), 3.63 (s, 1H), 3.48 (dd, 1H), 2.46 (s, 1H), 2.25 (s, 2H), 2.19-2.06 (m, 2H), 2.06-1.97 (m, 2H), 1.76 (d, 1H), 1.61 (s, 2H), 1.39-1.18 (m, 3H), 1.20-1.08 (m, 3H).

### Example 132

### (R)-1-[3-(Cyclohexyl-hydroxy-phenyl-methyl)-[1,2,4]oxadiazol-5-ylmethyl]-3-phenylsulfanylmethyl-1-azonia-bicyclo[2.2.2]octane; chloride

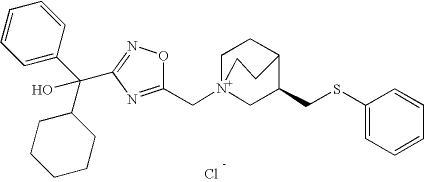

The title compound was prepared from Intermediate 20, Enantiomer 2 and the compound of Step 1, Example 100 by application of General Procedure F. Data for the title compound: LCMS (Method 6, Rt 8.85 min). M+=504. ¹H NMR (400 MHz, CHCl₃-d): δ 7.48-7.43 (m, 2H), 7.39-7.27 (m, 4H), 7.26-7.16 (m, 4H), 5.59 (d, 1H), 5.22 (d, 1H), 4.62 (s, 1H), 4.06-3.91 (m, 4H), 3.86-3.78 (m, 1H), 3.69-3.62 (m, 1H), 3.09-2.95 (m, 2H), 2.35-2.19 (m, 3H), 2.07 (d, 2H), 1.89 (s, 2H), 1.74 (d, 1H), 1.61 (s, 3H), 1.35-1.16 (m, 3H), 1.18-1.03 (m, 3H).

### Example 133

### (S)-1-[3-(Cyclohexyl-hydroxy-phenyl-methyl)-[1,2,4]oxadiazol-5-ylmethyl]-3-(3-fluoro-phenylsulfanylmethyl)-1-azonia-bicyclo[2.2.2]octane; chloride

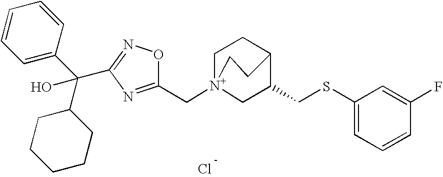

The title compound was prepared from Intermediate 20, Enantiomer 2 and the compound of Step 11 Example 111 by application of General Procedure F. Data for the title compound: LCMS (Method 6, Rt 9.01 min). M+=522. ¹H NMR (400 MHz, CHCl₃-d): δ 7.45-7.40 (m, 2H), 7.29 (d, 1H), 7.29-7.09 (m, 4H), 7.04 (dt, 1H), 6.91 (td, 1H), 5.62 (d, 1H), 5.04 (d, 1H), 4.95 (s, 1H), 4.11-3.99 (m, 3H), 3.92 (d, 1H), 3.81-3.72 (m, 1H), 3.61 (dd, 1H), 3.13-2.99 (m, 2H), 2.38-2.17 (m, 3H), 2.13-2.07 (m, 2H), 1.95-1.83 (m, 2H), 1.68-1.54 (m, 4H), 1.34-1.16 (m, 3H), 1.18-0.99 (m, 3H).

### Example 134

### (R)-1-[5-((R)—Cyclohexyl-hydroxy-phenyl-methyl)-[1,3,4]oxadiazol-2-ylmethyl]-3-(3-fluoro-phenoxy)-1-azonia-bicyclo[2.2.2]octane; bromide

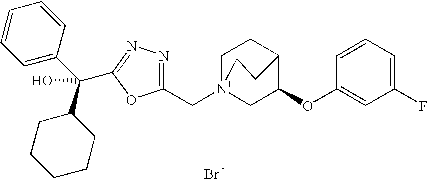

The title compound was prepared from Intermediate 18 and Intermediate 24 by application of General Procedure F. Data for the title compound: LCMS (Method 6, Rt 8.28 min). M+=492. ¹H NMR (400 MHz, DMSO-d₆): δ 7.49-7.44 (m, 2H), 7.39-7.24 (m, 5H), 6.91-6.78 (m, 3H), 6.43 (s, 1H), 4.99-4.90 (m, 3H), 4.00 (dd, 1H), 2.45 (s, 1H), 2.27 (t, 1H), 2.15 (s, 2H), 2.10-2.00 (m, 1H), 1.98-1.82 (m, 3H), 1.71 (s, 3H), 1.68-1.53 (m, 3H), 1.36 (d, 1H), 1.29-1.08 (m, 3H), 1.09-0.91 (m, 2H).

### Example 135

### (R)-3-(Benzo[1,3]dioxol-5-yloxy)-1-[3-hydroxy-diphenyl-methyl)-[1,2,4]oxadiazol-5-ylmethyl]-1-azonia-bicyclo[2.2.2]octane; chloride

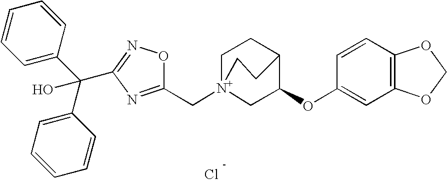

The title compound was prepared from Intermediate 22 and Intermediate 30 by application of General Procedure F. Data for the title compound: LCMS (Method 6, Rt 7.74 min). M+=512. ¹H NMR (400 MHz, DMSO-d₆): δ 7.41-7.25 (m, 10H), 7.14 (s, 1H), 6.84 (d, 1H), 6.71 (d, 1H), 6.41 (dd, 1H), 5.99 (s, 2H), 5.05 (s, 2H), 4.81 (s, 1H), 4.08-3.99 (m, 1H), 3.67-3.46 (m, 5H), 2.41 (s, 1H), 2.16 (s, 1H), 2.07-1.98 (m, 1H), 1.92-1.82 (m, 2H).

### Example 136

### (R)-3-(4-Chloro-phenoxy)-1-[3-(hydroxy-diphenyl-methyl)-[1,2,4]oxadiazol-5-ylmethyl]-1-azonia-bicyclo[2.2.2]octane; chloride

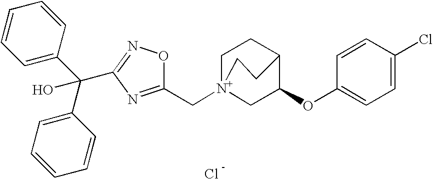

The title compound was prepared from Intermediate 22 and the compound of Step 1, Example 63 by application of General Procedure F. Data for the title compound: LCMS (Method 6, Rt 8.20 min). M+=502. ¹H NMR (400 MHz, DMSO-d₆): δ 7.40-7.26 (m, 12. H), 7.15 (s, 1H), 7.02-6.97 (m, 2H), 5.12-5.00 (m, 2H), 4.94 (s, 1H), 4.13-4.04 (m, 1H), 3.72-3.49 (m, 5H), 2.45 (s, 1H), 2.15 (s, 1H), 2.10-2.01 (m, 1H), 1.99-1.82 (m, 2H).

### Example 137

### (R)-1-[3-(Hydroxy-diphenyl-methyl)-[1,2,4]oxadiazol-5-ylmethyl]-3-(3-trifluoromethyl-phenoxy)-1-azonia-bicyclo[2.2.2]octane; chloride

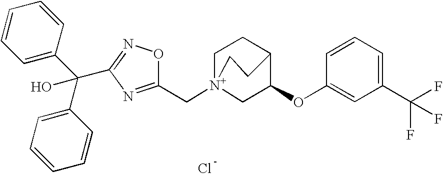

The title compound was prepared from Intermediate 22 and Intermediate 26 by application of General Procedure F. Data for the title compound: LCMS (Method 6, Rt 8.49 min). M+=536. ¹H NMR (400 MHz, DMSO-d₆): δ 7.58 (t, 1H), 7.39-7.25 (m, 13H), 7.14 (s, 1H), 5.08 (d, 3H), 4.13 (dd, 1H), 3.73-3.50 (m, 5H), 2.47 (s, 1-H), 2.18 (s, 1H), 2.10-2.01 (m, 1H), 2.01-1.83 (m, 2H).

### Example 138

### (R)-3-Cyclohexylsulfanyl-1-[3-(hydroxy-diphenyl-methyl)-[1,2,4]oxadiazol-5-ylmethyl]-1-azonia-bicyclo[2.2.2]octane; chloride

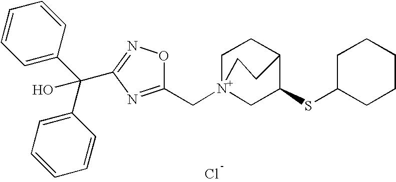

The title compound was prepared from Intermediate 22 and the compound of Step 1, Example 119 by application of General Procedure F. Data for the title compound: LCMS (Method 6, Rt 8.54 min). M+=490. ¹H NMR (400 MHz, DMSO-d₆): δ 7.42-7.32 (m, 7H), 7.32-7.26 (m, 3H), 7.13 (s, 1H), 4.95 (s, 2H), 4.03 (t, 1H), 3.61-3.42 (m, 5H), 3.41-3.33 (m, 1H), 2.79 (t, 1H), 2.18-2.03 (m, 2H), 2.02 (s, 2H), 1.88 (s, 3H), 1.68 (s, 2H), 1.56 (d, 1H), 1.37-1.17 (m, 5H).

### Example 139

### (R)-3-(3-Cyano-phenoxy)-1-[3-(hydroxy-diphenyl-methyl)-[1,2,4]oxadiazol-5-ylmethyl]-1-azonia-bicyclo[2.2.2]octane; chloride

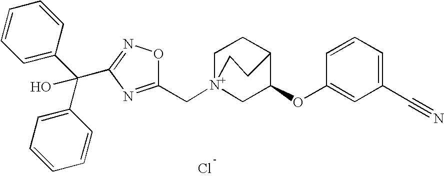

The title compound was prepared from Intermediate 22 and Intermediate 31 by application of General Procedure F. Data for the title compound: LCMS (Method 6, Rt 7.57 min). M+=493. ¹H NMR (400 MHz, DMSO-d₆): δ 7.56-7.45 (m, 3H), 7.40-7.36 (m, 4H), 7.35-7.25 (m, 7H), 7.17 (s, 1H), 5.15-4.97 (m, 3H), 4.15 (dd, 1H), 3.72-3.53 (m, 5H), 2.49-2.46 (m, 1H), 2.19-1.97 (m, 2H), 2.01-1.83 (m, 2H).

### Example 140

### (R)-1-[5-((R)—Cyclohexyl-hydroxy-phenyl-methyl)-[1,3,4]oxadiazol-2-ylmethyl]-3-(4-fluoro-benzyloxy)-1-azonia-bicyclo[2.2.2]octane; bromide

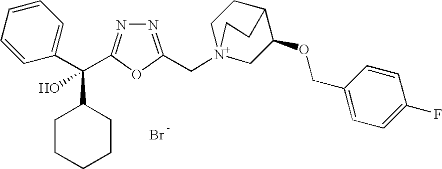

The title compound was prepared from Intermediate 18 and the compound of Step 1, Example 32 by application of General Procedure F. Data for the title compound: LCMS (Method 6, Rt 8.44 min). M+=506. ¹H NMR (400 MHz, DMSO-d₆): δ 7.49-7.44 (m, 2H), 7.42-7.31 (m, 4H), 7.30-7.25 (m, 1H), 7.22-7.15 (m, 2H), 6.43 (s, 1H), 4.91 (s, 2H), 4.54-4.41 (m, 2H), 3.99 (s, 1H), 3.82-3.73 (m, 1H), 3.38 (d, 4H), 2.41 (s, 1H), 2.25 (t, 1H), 2.14-1.90 (m, 2H), 1.85-1.74 (m, 2H), 1.73-1.53 (m, 4H), 1.34 (d, 1H), 1.27-1.09 (m, 3H), 1.07-0.92 (m, 3H).

### Example 141

### (R)-3-(4-Fluoro-benzyloxy)-1-[3-(hydroxy-diphenyl-methyl)-[1,2,4]oxadiazol-5-ylmethyl]-1-azonia-bicyclo[2.2.2]octane; chloride

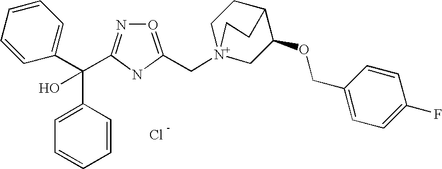

The title compound was prepared from Intermediate 22 and the compound of Step 1, Example 32 by application of General Procedure F. Data for the title compound: LCMS (Method 6, Rt 8.02 min). M+=500. ¹H NMR (400 MHz, DMSO-d₆): δ 7.42-7.25 (m, 12H), 7.22-7.15 (m, 2H), 7.13 (s, 1H), 5.01 (s, 2H), 4.56-4.44 (m, 2H), 4.01 (d, 1H), 3.92-3.83 (m, 1H), 3.66-3.43 (m, 5H), 2.43 (s, 1H), 2.13-1.92 (m, 2H), 1.87-1.76 (m, 2H).

### Example 142

### (R)-1-[5-((R)—Cyclohexyl-hydroxy-phenyl-methyl)-[1,3,4]oxadiazol-2-ylmethyl]-3-(4-fluoro-phenoxy)-1-azonia-bicyclo[2.2.2]octane; bromide

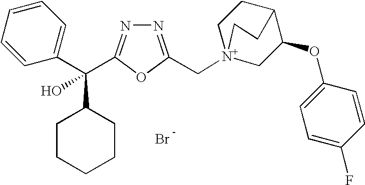

The title compound was prepared from Intermediate 18 and Intermediate 23 by application of General Procedure F. Data for the title compound: LCMS (Method 6, Rt 8.29 min). M+=492. ¹H NMR (400 MHz, DMSO-d₆): δ 7.44-7.39 (m, 2H), 7.34-7.21 (m, 3H), 7.16-7.09 (m, 2H), 6.97-6.90 (m, 2H), 6.39 (s, 1H), 4.92 (s, 2H), 4.82 (s, 1H), 3.97-3.87 (m, 1H), 3.59-3.37 (m, 5H), 2.38 (s, 1H), 2.22 (t, 1H), 2.11 (s, 1H), 2.00 (s, 1H), 1.84 (s, 2H), 1.66 (s, 2H), 1.57 (t, 2H), 1.32 (d, 1H), 1.23-1.00 (m, 3H), 1.03-0.88 (m, 2H).

### Example 143

### (R)-1-[3-(Cyclohexyl-hydroxy-phenyl-methyl)-isoxazol-5-ylmethyl]-3-(4-fluoro-phenoxy)-1-azonia-bicyclo[2.2.2]octane; chloride

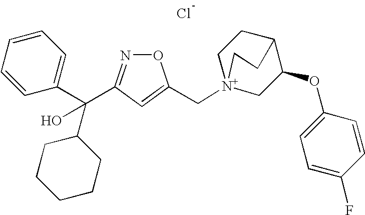

The title compound was prepared from Intermediate 21, Enantiomer 2 and Intermediate 23 by application of General Procedure F. Data for the title compound: LCMS (Method 6, Rt 8.68 min). M+=491. ¹H NMR (400 MHz, DMSO-d₆): δ 7.51-7.46 (m, 2H), 7.32 (t, 2H), 7.25-7.12 (m, 3H), 7.02-6.95 (m, 2H), 6.79 (s, 1H), 5.90 (s, 1H), 4.88 (s, 1H), 4.77 (s, 2H), 3.91 (dd, 1H), 3.54-3.34 (m, 5H), 2.39 (s, 1H), 2.24-2.09 (m, 2H), 2.06-1.97 (m, 1H), 1.94-1.80 (m, 2H), 1.68 (d, 1H), 1.58 (d, 3H), 1.28-1.13 (m, 3H), 1.10-0.98 (m, 3H).

### Example 144

### (S)-1-[3-(Cyclohexyl-hydroxy-phenyl-methyl)-[1,2,4]oxadiazol-5-ylmethyl]-3-phenoxymethyl-1-azonia-bicyclo[2.2.2]octane; chloride

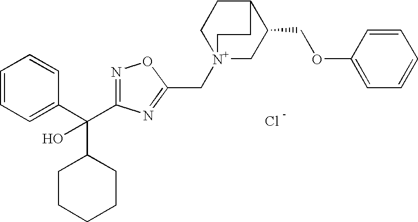

The title compound was prepared from Intermediate 20, Enantiomer 2 and the compound of Step 1 (Method 2), Example 24 by application of General Procedure F. Data for the title compound: LCMS (Method 6, Rt 8.71 min). M+=488. ¹H NMR (400 MHz, CHCl₃-d): δ 7.42-7.37 (m, 2H), 7.30-7.23 (m, 2H), 7.29-7.11 (m, 3H), 6.99-6.92 (m, 1H), 6.86 (d, 2H), 5.61 (d, 1H), 5.01 (d, 1H), 4.88 (s, 1H), 4.22 (t, 1H), 4.09 (s, 1H), 4.03-3.91 (m, 3H), 3.85 (s, 1H), 3.63-3.55 (m, 1H), 2.69 (s, 1H), 2.24 (d, 3H), 2.07 (s, 2H), 1.92 (d, 1H), 1.71 (s, 5H), 1.36-1.15 (m, 3H), 1.17-1.02 (m, 3H).

### BIOLOGICAL EXAMPLES

The inhibitory effects of compounds of the present invention at the M3 muscarinic receptor were determined by the following binding assays:

### Muscarinic Receptor Radioligand Binding Assays

Radioligand binding studies utilising [³H]-N-methyl scopolamine ([³H]-NMS) and commercially available cell membranes expressing the human muscarinic receptors (M2 and M3) were used to assess the affinity of muscarinic antagonists for M2 and M3 receptors. Membranes in TRIS buffer were incubated in 96-well plates with [³H]-NMS and M3 antagonist at various concentrations for 3 hours. Membranes and bound radioligand were then harvested by filtration and allowed to dry overnight. Scintillation fluid was then added and the bound radioligand counted using a Canberra Packard Topcount scintillation counter

The half-life of antagonists at each muscarinic receptor was measured using the alternative radioligand [³H]-QNB and an adaptation of the above affinity assay. Antagonists were incubated for 3 hours at a concentration 10-fold higher than their Ki, as determined with the [³H]-QNB ligand, with membranes expressing the human muscarinic receptors. At the end of this time, [³H]-QNB was added to a concentration 25-fold higher than its Kd for the receptor being studied and the incubation continued for various time periods from 15 minutes up to 180 minutes. Membranes and bound radioligand were then harvested by filtration and allowed to dry overnight. Scintillation fluid was then added and the bound radioligand counted using a Canberra Packard Topcount scintillation counter.

The rate at which [3H]-QNB is detected binding to the muscarinic receptors is related to the rate at which the antagonist dissociates from the receptor, ie. to the half life of the antagonists on the receptors.

Alternatively,

Recombinant human M3 receptor was expressed in CHO-K1 cells. Cell membranes were prepared and binding of [3H]-N-methyl scopolamine ([3H]-NMS) and compounds was assessed by a scintillation proximity assay (SPA). The incubation time was 16 hours at room temperature in the presence of 1% (v/v) DMSO. The assay was performed in white 96 well clear-bottomed NBS plates (Corning). Prior to the assay, the CHO cell membranes containing M3 receptor were coated onto SPA WGA (Wheat germ agglutinin) beads (GE Healthcare). Non specific binding was determined in the presence of 1 μM Atropine.

Radioactivity was measured on a Microbeta scintillation counter (PerkinElmer) using a 3H protocol with a 2 minutes per well read time. Compound inhibition of [3H]-NMS binding was determined typically using concentrations in the range 0.03 nM to 1 μM and expressed as percent inhibition relative to the plate specific radioligand binding for the plate. Concentration dependent inhibition of [3H]-NMS binding by compounds was expressed as pIC50.

Binding data for Examples of the invention, where tested, are shown in the table below.

| | M3 Ki | |
|---|---|---|
| | (Filter | M3 Ki |
| | plate | (SPA |
| Example | format) | format |
| 1 | + | + |
| 2 | +++ | +++ |
| 3 | ++ | |
| 4 | +++ | |
| 5 | | +++ |
| 6 | | + |
| 7 | | ++ |
| 8 | | ++ |
| 9 | | +++ |
| 10 | ++ | ++ |
| 11 | + | + |
| 12 | | +++ |
| 13 | | + |
| 14 | | ++ |
| 15 | | +++ |
| 16 | | +++ |
| 17 | | + |
| 18 | | + |
| 19 | | +++ |
| 20 | | +++ |
| 21 | | +++ |
| 22 | | +++ |
| 23 | | +++ |
| 24 | | +++ |
| 25 | | +++ |
| 26 | | +++ |
| 27 | | +++ |
| 28 | | ++ |
| 29 | | +++ |
| 30 | | +++ |
| 31 | | +++ |
| 32 | | +++ |
| 33 | | +++ |
| 34 | | +++ |
| 35 | | +++ |
| 36 | | +++ |
| 37 | | +++ |
| 38 | | +++ |
| 39 | | ++ |
| 40 | | +++ |
| 41 | | +++ |
| 42 | | +++ |
| 43 | | +++ |
| 44 | | +++ |
| 45 | | +++ |
| 46 | | +++ |
| 47 | | + |
| 48 | | +++ |
| 49 | | +++ |
| 50 | | +++ |
| 51 | | +++ |
| 52 | | +++ |
| 53 | | +++ |
| 54 | | +++ |
| 55 | | +++ |
| 56 | | +++ |
| 57 | | ++ |
| 58 | | +++ |
| 59 | | +++ |
| 60 | | +++ |
| 61 | | +++ |
| 62 | | +++ |
| 63 | | +++ |
| 64 | | +++ |
| 65 | | +++ |
| 66 | | +++ |
| 67 | | +++ |
| 68 | | +++ |
| 69 | | +++ |
| 70 | | +++ |
| 71 | | +++ |
| 72 | | +++ |
| 73 | | +++ |
| 74 | | + |
| 75 | | +++ |
| 76 | | +++ |
| 77 | | + |
| 78 | | +++ |
| 79 | | +++ |
| 80 | | +++ |
| 81 | | +++ |
| 82 | | +++ |
| 83 | | ++ |
| 84 | | +++ |
| 85 | | +++ |
| 86 | | ++ |
| 87 | | +++ |
| 88 | | ++ |
| 89 | | +++ |
| 90 | | ++ |
| 91 | | +++ |
| 92 | | ++ |
| 93 | | +++ |
| 94 | | +++ |
| 95 | | +++ |
| 96 | | +++ |
| 97 | | +++ |
| 98 | | +++ |
| 99 | | +++ |
| 100 | | +++ |
| 101 | | +++ |
| 102 | | ++ |
| 103 | | +++ |
| 104 | | +++ |
| 105 | | +++ |
| 106 | | +++ |
| 107 | | +++ |
| 108 | | +++ |
| 109 | | +++ |
| 110 | | +++ |
| 111 | | +++ |
| 112 | | +++ |
| 113 | | +++ |
| 114 | | ++ |
| 115 | | +++ |
| 116 | | +++ |
| 117 | | +++ |
| 118 | | +++ |
| 119 | | +++ |
| 120 | | +++ |
| 121 | | +++ |
| 122 | | +++ |
| 123 | | +++ |
| 124 | | +++ |
| 125 | | ++ |
| 126 | | +++ |
| 127 | | +++ |
| 128 | | +++ |
| 129 | | +++ |
| 130 | | +++ |
| 131 | | +++ |
| 132 | | +++ |
| 133 | | +++ |
| 134 | | +++ |
| 135 | | +++ |
| 136 | | +++ |
| 137 | | +++ |
| 138 | | +++ |
| 139 | | +++ |
| 140 | | +++ |
| 141 | | +++ |
| 142 | | +++ |
| 143 | | +++ |
| 144 | | +++ |

M3 Kᵢ <5 nM “+++”; 5-20 nM “++”, >20 nM “+”

All compounds tested exhibited potencies (as Kᵢ values) in the M3 binding assay of greater than 1 μM. In particular, Example 4 exhibited a Kᵢ value of 2.0 nM and Example 2 exhibited a Kᵢ value of 0.9 nM in the filter plate assay, and Example 42 exhibited a Kᵢ of 0.09 nM, Example 66 exhibited a Kᵢ of 0.09 nM, Example 25 exhibited a K of 0.13 nM, Example 70 exhibited a Kᵢ of 0.65 nM and Example 95 exhibited a Kᵢ of 0.17 nM in the SPA format assay.

### Analysis of Inhibition of M3 Receptor Activation via Calcium Mobilization

CHO cells expressing the human M3 receptor were seeded and incubated overnight in 96 well collagen coated plates (black-wall, clear bottom) at a density of 50000/75 μl of medium in 3% serum. The following day, a calcium-sensitive dye (Molecular Devices, Cat # R8041) was prepared in HBSS buffer with the addition of 5 mM probenecid (pH 7.4). An equal volume of the dye solution (75 μl) was added to the cells and incubated for 45 minutes followed by addition of 50 μl of muscarinic antagonists or vehicle. After a further 15 minutes the plate was read on a FLEXstation™ (excitation 488 nm, emission 525 nm) for 15 seconds to determine baseline fluorescence. The muscarinic agonist Carbachol was then added at an EC₈₀ concentration and the fluorescence measured for a further 60 seconds. The signal was calculated by subtracting the peak response from the mean of the baseline fluorescence in control wells in the absence of antagonist. The percentage of the maximum response in the presence of antagonist was then calculated in order to generate IC₅₀ curves

### Evaluation of Potency and Duration of Action in Isolated Guinea Pig Trachea

Experiments were carried out at 37° C. in modified Krebs-Henseleit solution, (114 mM NaCl, 15 mM NaHCO₃, 1 mM MgSO₄, 1.3 mM CaCl₂, 4.7 mM KCl, 11.5 mM glucose and 1.2 mM KH₂PO₄, pH 7.4) gassed with 95% O₂/5% CO₂. Indomethacin was added to a final concentration of 3 μM

Tracheae were removed from adult male Dunkin Hartley Guinea pigs and dissected free of adherent tissue before being cut open longitudinally in a line opposite the muscle. Individual strips of 2-3 cartilage rings in width were cut and suspended using cotton thread in 10 mL water-jacketed organ baths and attached to a force transducer ensuring that the tissue is located between two platinum electrodes. Responses were recorded via a MP100W/Acknowledge data acquisition system connected to a PC. Tissues were equilibrated for one hour under a resting tone of 1 g and were then subjected to electrical field stimulation at a frequency of 80 Hz with a pulse width of 0.1 ms, a unipolar pulse, triggered every 2 minutes. A “voltage-response” curve was generated for each tissue and a submaximal voltage then applied to every piece of tissue according to its own response to voltage. Tissues were washed with Krebs solution and allowed to stabilize under stimulation prior to addition of test compound. Concentration response curves were obtained by a cumulative addition of test compound in half-log increments. Once the response to each addition had reached a plateau the next addition was made. Percentage inhibition of EFS-stimulated contraction is calculated for each concentration of each compound added and dose response curves constructed using Graphpad Prism software and the IC₅₀ calculated for each compound. By means of further exemplification, Example 25 had an IC₅₀ of 1.9 nM, Example 42 had an IC₅₀ of 0.7 nM, Example 70 had an IC₅₀ of 2.8 nM and Example 95 had an IC₅₀ of 0.6 nM in this assay.

Onset time and duration of action studies were performed by adding the previously determined EC₅₀ concentration of compound to EFS contracted tissues and the response allowed to plateau. The time taken to reach 50% of this response was determined to be the onset time. Tissues were then washed free of compound by flushing the tissue bath with fresh Krebs solution and the time taken for the contraction in response to EFS to return to 50% of the response in the presence of compound is measured. This is termed the duration of action.

### Methacholine Induced Bronchoconstriction In Vivo

Male Guinea pigs (Dunkin Hartley), weighing 500-600 g housed in groups of 5 were individually identified. Animals were allowed to acclimatize to their local surroundings for at least 5 days. Throughout this time and study time animals were allowed access to water and food ad libitum.

Guinea pigs were anaesthetized with the inhaled anaesthetic Halothane (5%). Test compound or vehicle (0.25-0.50 mL/kg) was administered intranasally. Animals were placed on a heated pad and allowed to recover before being returned to their home cages.

Up to 72 hrs post dosing guinea pigs were terminally anaesthetized with Urethane (250 μg/mL, 2 mL/kg). At the point of surgical anesthesia, the jugular vein was cannulated with a portex i.v. cannula filled with heparinised phosphate buffered saline (hPBS) (10 U/mL) for i.v. administration of methacholine. The trachea was exposed and cannulated with a rigid portex cannula and the esophagus cannulated transorally with a flexible portex infant feeding tube.

The spontaneously breathing animal was then connected to a pulmonary measurement system (EMMS, Hants, UK) consisting of a flow pneumotach and a pressure transducer.

The tracheal cannula was attached to a pneumotach and the oesophageal cannula attached to a pressure transducer.

The oesophageal cannula was positioned to give a baseline resistance of between 0.1 and 0.2 cmH20/mL/s. A 2 minute baseline reading was recorded before i.v. administration of methacholine (up to 30 μg/kg, 0.5 mL/kg). A 2 minute recording of the induced constriction was taken from the point of i.v. administration.

The software calculated a peak resistance and a resistance area under the curve (AUC) during each 2 minute recording period which were used to analyze the bronchoprotective effects of test compounds.

Inhibition of Pilocarpine Induced Salivation by i.n. Administered Compounds

Guinea pigs (450-550 g) supplied by Harlan UK or David Hall, Staffs UK and acclimatized to the in-house facilities for a minimum of three days before use. Guinea pigs were randomly assigned into treatment groups and weighed. Each animal was lightly anaesthetized (4% Halothane) and administered compound or vehicle intranasally (0.5 mL/kg) at up to 24 hours before challenge with pilocarpine. At the test time point, guinea pigs were terminally anaesthetized with urethane (25% solution in H₂O, 1.5 g/kg). Once sufficient anesthesia had developed (absence of toe pinch reflex) each animal had an absorbent pad placed in the mouth for 5 minutes to dry residual saliva, this pad was removed and replaced with a new pre-weighed pad for 5 minutes to establish a reading of baseline saliva production. At the end of this 5 minute period the pad was removed and weighed. A new pre-weighed pad was inserted into the mouth before each animal received s.c. pilocarpine administered under the skin at the back of the neck (0.6 mg/kg @ 2 mL/kg). The pad was removed, weighed and replaced with a new pre-weighed pad every 5 minutes up to 15 minutes.

Saliva production was calculated by subtracting the pre-weighed weight of the pad from each 5 minute period post weighed pad and these numbers added together to produce an accumulation of saliva over 15 minutes. Each 5 minute period could be analyzed in addition to the whole 15 minute recording period. Baseline production of saliva was assumed to be constant and multiplied by three to produce a reading for baseline saliva production over 15 minutes.

Inhibition of saliva produced by the compound could be calculated by using the following equation:

1-(Test−baseline)/(Veh−baseline))*100.

1-8. (canceled)

9. A compound or a pharmaceutically acceptable salt thereof, wherein the compound is
(R)-1-[3-(Cyclohexyl-hydroxy-phenyl-methyl)-isoxazol-5-ylmethyl]-3-(3-fluoro-phenoxy)-1-azonia-bicyclo[2.2.2]octane

10. (canceled)

11. A pharmaceutical composition, wherein the composition comprises:
the compound or a salt thereof as claimed in claim 9, and
a pharmaceutically acceptable carrier or excipient.

12. A pharmaceutical composition as claimed in claim 11, wherein the composition is in a form suitable for inhalation.

13. (canceled)

14. A method of treatment of a disease or condition in which M3 muscarinic receptor activity is implicated, wherein the method comprises administration to a subject in need thereof of a therapeutically effective amount of a compound or a salt thereof as claimed in claim 9.

15. A method of treatment as claimed in claim 14, wherein the disease or condition is a respiratory-tract disorder.

16. A method of treatment as claimed in claim 14, wherein the disease or condition is a gastrointestinal-tract disorder.

17. A method of treatment as claimed in claim 14, wherein the disease or condition is a cardiovascular disorder.

18. A method of treatment as claimed in claim 14, wherein the disease or condition is chronic obstructive lung disease, chronic bronchitis, asthma, adult/acute respiratory distress syndrome, chronic respiratory obstruction, bronchial hyperactivity, pulmonary fibrosis, pulmonary emphysema, or allergic rhinitis.

19. A method of treatment as claimed in claim 14, wherein the disease or condition is irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakiuria, neurogenic bladder, nocturnal enuresis, psychosomatic bladder, incontinence associated with bladder spasms or chronic cystitis, urinary urgency or pollakiuria, or motion sickness.

20. A method of treatment as claimed in claim 14, wherein the disease or condition is vagally induced sinus bradycardia.

21. A salt as claimed in claim 9, wherein the salt comprises a chloride, bromide, sulfate, methanesulfonate, benzenesulfonate, toluenesulfonate, napadisylate, edisylate, isethionate, xinafoate, p-acetamidobenzoate, phosphate, acetate, citrate, lactate, tartrate, maleate, fumarate, acetamidobenzoate, or succinate salt.

22. A pharmaceutical composition, wherein the composition comprises:
a salt claimed in claim 21, and
a pharmaceutically acceptable carrier or excipient.

23. A pharmaceutical composition as claimed in claim 22, wherein the composition is in a form suitable for inhalation.

24. A method of treatment of a disease or condition in which M3 muscarinic receptor activity is implicated, wherein the method comprises administration to a subject in need thereof of a therapeutically effective amount of a salt claimed in claim 21.

25. A method of treatment as claimed in claim 24, wherein the disease or condition is a respiratory-tract disorder.

26. A method of treatment as claimed in claim 24, wherein the disease or condition is a gastrointestinal-tract disorder.

27. A method of treatment as claimed in claim 24, wherein the disease or condition is a cardiovascular disorder.

28. A method of treatment as claimed in claim 24, wherein the disease or condition is chronic obstructive lung disease, chronic bronchitis, asthma, adult/acute respiratory distress syndrome, chronic respiratory obstruction, bronchial hyperactivity, pulmonary fibrosis, pulmonary emphysema, or allergic rhinitis.

29. A method of treatment as claimed in claim 24, wherein the disease or condition is irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakiuria, neurogenic bladder, nocturnal enuresis, psychosomatic bladder, incontinence associated with bladder spasms or chronic cystitis, urinary urgency or pollakiuria, or motion sickness.

30. A method of treatment as claimed in claim 24, wherein the disease or condition is vagally induced sinus bradycardia.

31. A compound or a pharmaceutically acceptable salt thereof, wherein the compound is (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane.

32. A pharmaceutical composition, wherein the composition comprises:
the compound or a salt thereof as claimed in claim 31, and
a pharmaceutically acceptable carrier or excipient.

33. A pharmaceutical composition as claimed in claim 32, wherein the composition is in a form suitable for inhalation.

34. A method of treatment of a disease or condition in which M3 muscarinic receptor activity is implicated, wherein the method comprises administration to a subject in need thereof of a therapeutically effective amount of a compound or a salt thereof as claimed in claim 31.

35. A method of treatment as claimed in claim 34, wherein the disease or condition is a respiratory-tract disorder.

36. A method of treatment as claimed in claim 34, wherein the disease or condition is a gastrointestinal-tract disorder.

37. A method of treatment as claimed in claim 34, wherein the disease or condition is a cardiovascular disorder.

38. A method of treatment as claimed in claim 34, wherein the disease or condition is chronic obstructive lung disease, chronic bronchitis, asthma, adult/acute respiratory distress syndrome, chronic respiratory obstruction, bronchial hyperactivity, pulmonary fibrosis, pulmonary emphysema, or allergic rhinitis.

39. A method of treatment as claimed in claim 34, wherein the disease or condition is irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakiuria, neurogenic bladder, nocturnal enuresis, psychosomatic bladder, incontinence associated with bladder spasms or chronic cystitis, urinary urgency or pollakiuria, or motion sickness.

40. A method of treatment as claimed in claim 34, wherein the disease or condition is vagally induced sinus bradycardia.

41. A salt claimed in claim 31, wherein the salt comprises a chloride, bromide, sulfate, methanesulfonate, benzenesulfonate, toluenesulfonate, napadisylate, edisylate, isethionate, xinafoate, p-acetamidobenzoate, phosphate, acetate, citrate, lactate, tartrate, maleate, fumarate, acetamidobenzoate, or succinate salt.

42. A pharmaceutical composition, wherein the composition comprises:
a salt as claimed in claim 41, and
a pharmaceutically acceptable carrier or excipient.

43. A pharmaceutical composition as claimed in claim 42, wherein the composition is in a form suitable for inhalation.

44. A method of treatment of a disease or condition in which M3 muscarinic receptor activity is implicated, wherein the method comprises administration to a subject in need thereof of a therapeutically effective amount of a salt claimed in claim 41.

45. A method of treatment as claimed in claim 44, wherein the disease or condition is a respiratory-tract disorder.

46. A method of treatment as claimed in claim 44, wherein the disease or condition is a gastrointestinal-tract disorder.

47. A method of treatment as claimed in claim 44, wherein the disease or condition is a cardiovascular disorder.

48. A method of treatment as claimed in claim 44, wherein the disease or condition is chronic obstructive lung disease, chronic bronchitis, asthma, adult/acute respiratory distress syndrome, chronic respiratory obstruction, bronchial hyperactivity, pulmonary fibrosis, pulmonary emphysema, or allergic rhinitis.

49. A method of treatment as claimed in claim 44, wherein the disease or condition is irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakiuria, neurogenic bladder, nocturnal enuresis, psychosomatic bladder, incontinence associated with bladder spasms or chronic cystitis, urinary urgency or pollakiuria, or motion sickness.

50. A method of treatment as claimed in claim 44, wherein the disease or condition is vagally induced sinus bradycardia.
